(19) United States
(12) Patent Application Publication
(10) Pub. No.: US 2025/0064917 A1
(43) Pub. Date: Feb. 27, 2025

(54) HVT AIV VECTORS AND USES THEREOF

(71) Applicant: Boehringer Ingelheim Vetmedica GmbH, Ingelheim am Rhein (DE)

(72) Inventors: Teshome Mebatsion, Acworth, GA (US); Michel Bublot, Chaponost (FR); Frederic Reynard, Saint-Bonnet-de-Mure (FR)

(21) Appl. No.: 18/812,062

(22) Filed: Aug. 22, 2024

Related U.S. Application Data
(60) Provisional application No. 63/578,877, filed on Aug. 25, 2023.

(51) Int. Cl. A61K 39/145; A61K 39/00; A61K 39/17; A61P 31/16; C12N 7/00; C12N 15/86

(52) U.S. Cl. CPC A61K 39/145; A61K 39/17; A61P 31/16; C12N 7/00; C12N 15/86; A61K 2039/54; A61K 2039/545; A61K 2039/552; A61K 2039/575; C12N 2710/16322; C12N 2710/16343; C12N 2710/16351; C12N 2760/16134; C12N 2800/22; C12N 2840/203

(57) ABSTRACT

The present invention provides recombinant herpes virus of turkeys (HVT) viral vectors comprising a heterologous polynucleotide encoding an avian influenza antigen. The recombinant viral vectors are suitable for use in immunogenic compositions and vaccines, and can provide protection against avian influenza and other pathogens when administered to an avian.

### SEQUENCE LISTING

This application contains a Sequence Listing in accordance with 37 C.F.R. 1.821-1.825. The Sequence Listing associated with this application is submitted electronically as an XML file, compliant with WIPO Standard ST.26. The XML file, titled “23-0046-US-2.xml,” created on Jul. 19, 2024, and 82,194 byte in size, is incorporated herein by reference in its entirety.

### FIELD OF THE INVENTION

The present invention relates to recombinant turkey herpesvirus (HVT) viral vectors comprising a foreign avian influenza virus (AIV) H9 haemagglutinin (AIV H9-HA) gene. The recombinant viral vectors are suitable for use in immunogenic compositions and vaccines, and can provide protection against AIV and other avian pathogens, particularly Marek's disease virus (MDV) as well as infectious bursal disease virus (IBDV) and Newcastle disease virus (NDV).

### BACKGROUND OF THE INVENTION

Avian influenza H9N2 virus is considered a low-pathogenic virus that is endemic to poultry populations. Low pathogenicity avian influenza has adverse effects on poultry production and poses a significant cross-species transmission and zoonotic threat. Thus, the development of a highly effective influenza virus vaccine for poultry would be of benefit to both human and veterinary health.

Marek's disease virus (MDV) belongs to the family alphaherpesvirideae and the genus Mardivirus. Among the serotypes of MDV are MDV serotype 1 (MDV1) and MDV serotype 2 (MDV2) which are both pathogenic to poultry, while MDV serotype 3 (MDV3) is not. In some instances, MDV3 is referred to as: Meleagrid herpesvirus 1, turkey herpesvirus, and/or herpesvirus of turkeys (HVT).

Different strains of MDV3 (i.e., HVT), such as PB 1 or FC-126, have been used to vaccinate avians (e.g., chickens) against MD caused by MDV1 and/or MDV2. Several recombinant HVT vectors expressing antigens from various pathogens (U.S. Pat. Nos. 5,980,906, 5,853,733, 6,183,753, and 5,187,087) have also been developed and licensed. Furthermore, there have been attempts in the art to develop recombinant HVT vectors expressing antigens from two different pathogens (multivalent recombinant HVT vectors), such as via the insertion of transgenes encoding infectious bursal disease virus (IBDV) and Newcastle disease virus (NDV) antigens into multiple different sites in the HVT vector genome (WO2013/144355). Similarly, there have been some preliminary attempts to provide recombinant HVT vectors encoding a heterologous AIV H9N2 antigen, in combination with a NDV and/or IBDV antigen, all of which have followed the same recombinant strategy of utilising multiple different insertion sites within the HVT vector genome (WO2022/079160, WO2021/123104).

The development of effective influenza virus vaccines for poultry would be of benefit to both human and veterinary health. The development of effective MDV, NDV and IBDV vaccines would also be of benefit. In particular, it would be of benefit to provide a combined vaccine against MDV and AIV, as well as an additional pathogen such as IBDV or NDV. It would be of benefit to provide such vaccines further to any attempts that have been made in the art. However, it is not always straightforward to predict how successful a particular recombinant strategy will be in a multivalent HVT-AIV H9 vector context, especially in the context of a HVT-AIV H9 vector that must express an additional heterologous antigen. Specifically, it is not straightforward to predict whether the use of multiple insertion sites or a single insertion site in the HVT genome will be successful for expressing an AIV H9 antigen and at least one additional antigen. It is also not straightforward to predict whether regulatory sequences such as IRES or P2A sequences will function properly in these circumstances. In particular, it is not straightforward to predict whether such novel combinations of these design choices and antigens will result in constructs with favourable stability, favourable expression of inserted heterologous genes and the provision of favourable heterologous protection against AIV, NDV and/or IBDV.

Citation or identification of any document in this application is not an admission that such document is available as prior art to the present invention.

### SUMMARY OF THE INVENTION

The invention generally provides recombinant HVT vectors comprising an avian influenza (AIV) H9 haemagglutinin antigen (AIV H9-HA, also referred to herein as AIV H9), as well as at least one additional avian antigen of an avian pathogen. The recombinant viral vectors can be used in immunogenic compositions and vaccines to provide animals with protection against avian influenza and MDV. The recombinant vectors include additional antigens from avian pathogenic organisms other than avian influenza, such as infectious bursal disease virus (IBDV) and Newcastle disease virus (NDV), to aid in the protection against additional pathogens beyond avian influenza.

In a first aspect, the present invention provides a recombinant herpesvirus of turkeys (HVT) vector comprising a first heterologous polynucleotide encoding a first avian antigen and a second heterologous polynucleotide encoding a second avian antigen, wherein the first and second heterologous polynucleotides are inserted in the same non-essential site in the recombinant HVT vector genome, and wherein the first or second antigen is an avian influenza virus (AIV) subtype H9 haemagglutinin (AIV H9-HA) antigen. Herein, the avian antigen that is not the AIV H9-HA antigen may be referred to as the “additional avian antigen”. Most preferably, the second antigen as defined in the present invention is the AIV H9-HA antigen. Most preferably, the first antigen as defined in the present invention is the additional avian antigen.

In a second aspect, the present invention provides a nucleic acid molecule comprising the recombinant HVT vector of the invention.

In a third aspect, the present invention provides an antigen expression cassette comprising the following elements:
(a) optionally a mCMV IE promoter;
(b) a first heterologous polynucleotide encoding a first avian antigen;
(c) optionally an IRES or P2A sequence;
(d) a second heterologous polynucleotide encoding a second avian antigen; and
(e) optionally an SV40 polyadenylation signal;
   wherein the first or second avian antigen is an AIV antigen, preferably an AIV H9-HA antigen.

Alternatively or additionally in a third aspect, the present invention provides an antigen expression cassette comprising in the following order 5′ to 3′:
(a) optionally a mCMV IE promoter;
(b) a first heterologous polynucleotide encoding a first avian antigen;
(c) optionally an IRES or P2A sequence;
(d) a second heterologous polynucleotide encoding a second avian antigen that is an AIV antigen, preferably an AIV H9-HA antigen; and
(e) optionally an SV40 polyadenylation signal.

In a fourth aspect, the present invention provides a cell comprising the recombinant HVT vector, the nucleic acid molecule or the expression cassette of the invention.

In a fifth aspect, the present invention provides a composition comprising the recombinant HVT vector, the nucleic acid molecule, the expression cassette or the cell of the invention.

In a sixth aspect, the present invention provides the recombinant HVT vector, the nucleic acid molecule, the expression cassette, the cell or the composition of the invention, for use in a method of inducing a protective immune response against AIV, the pathogen from which the additional (e.g. the first) avian antigen is derived and/or MDV in an avian.

In a seventh aspect, the present invention provides a method of inducing a protective immune response against AIV, the pathogen from which the additional (e.g. the first) avian antigen is derived and/or MDV in an avian, comprising administering the recombinant HVT vector, the nucleic acid molecule, the expression cassette, the cell or the composition of the invention to the avian.

In an eighth aspect, the present invention provides the recombinant HVT vector, the nucleic acid molecule, the expression cassette, the cell or the composition of the invention, for use in the manufacture of a medicament for inducing a protective immune response against AIV, the avian pathogen from which the additional (e.g. the first) avian antigen is derived and/or MDV in an avian.

In a ninth aspect, the present invention provides the recombinant HVT vector, the nucleic acid molecule, the expression cassette, the cell or the composition of the invention, for use in a method of reducing AIV shedding in an avian.

In a tenth aspect, the present invention provides a method of reducing AIV shedding in an avian, comprising administering the recombinant HVT vector, the nucleic acid molecule, the expression cassette, the cell or the composition of the invention to the avian.

In an eleventh aspect, the present invention provides the recombinant HVT vector, the nucleic acid molecule, the expression cassette, the cell or the composition of the invention, for use in the manufacture of a medicament for reducing AIV shedding in an avian.

In a twelfth aspect, the present invention provides a method of manufacturing the recombinant HVT vector of the invention, wherein the method comprises:
a) providing a nucleic acid encoding a HVT vector;
b) providing one or more expression cassettes comprising sequences encoding a first avian antigen, and a second avian antigen, wherein the first or second avian antigen is an AIV H9-HA antigen; and
c) recombinantly combining the nucleic acid encoding a HVT vector and the one or more expression cassettes, such that the sequences encoding the first avian antigen and the second avian antigen are comprised in the same non-essential site in the HVT vector genome.

### BRIEF DESCRIPTION OF THE DRAWINGS

FIG. 1 shows a diagram for constructing the donor plasmid pSaH9optIRESVP2 of Example 1.

FIG. 2 shows PCR primers' location on vHVT513 map.

FIGS. 3A-3B show a dual immunofluorescent assay. Representative pictures are from the pre-MSV passage (FIG. 3A) and from the pre-MSV+13 passage (FIG. 3B).

FIG. 4 shows a diagram for constructing the donor plasmid pFwtIRESSaH9opt of Example 2.

FIGS. 5A-5B show a dual immunofluorescent assay. Representative pictures are from the pre-MSV passage (FIG. 5A) and from the pre-MSV+13 passage (FIG. 5B).

FIG. 6 shows dispersions of individual global clinical scores by groups. G1: vHVT513; G2: vHVT514; G3: controls. Groups compared to CTL on the “GCS” criterion using a Mann-Whitney-Wilcoxon test. GCS was significantly lower in HVT groups than in the CTL group. Box plots represent the mini, the lower quartile, the median, the upper quartile and the maxi.

FIG. 7 shows H9N2 shedding after challenge. Left panel: mean oro-pharyngeal H9N2 RNA excretion by groups expressed as equivalent log 10 EID50/mM; the limit of detection was 2.09 log 10 equivalent EID50/ml. Right panel: percentage of birds positive for shedding at the three time points. Dpi=day post-infection (challenge).

FIG. 8 shows dispersions of individual AUC of viral RNA by groups; box plots represent the mini, the lower quartile, the median, the upper quartile and the maxi.

FIG. 9 shows dispersions of individual HAI titers against antigen IRAK on D20 by groups (log 10 HAI titers); box plots represent the mini, the lower quartile, the median, mean (cross), the upper quartile and the maxi. G1: vHVT513; G2: vHVT514; G3: controls. CTL group remained negative on D21. Seroconversion was observed in HVT groups.

FIG. 10 is a graph of the mean daily clinical score for vHVT513, vHVT514, Inactivated and Controls from 21 to 31 days post-vaccination.

### DETAILED DESCRIPTION

The recombinant HVT vectors of the invention can be used in immunogenic compositions and vaccines to provide animals with protection against avian influenza and MDV. In particular, the recombinant HVT vectors of the present invention provide an AIV vector vaccine (a recombinant HVT vector comprising a heterologous polynucleotide encoding an AIV H9-HA antigen) which is surprisingly effective even in stringent clinical conditions, with an unexpected level of efficacy and stability whilst providing protection against clinical signs. Due to the presence of an additional (e.g. the first) heterologous polynucleotide, the recombinant HVT vectors of the present invention can also provide protection against an additional heterologous avian pathogen, such as IBDV or NDV, while simultaneously providing protection against AIV. Benefits of the viral vectors of the invention include the capability to induce a protective immune response against MDV, AIV as well as such further avian pathogens, including a reduction in clinical signs, and a high level of viral stability. The present inventors have now surprisingly demonstrated that simultaneous protection against such pathogens can be achieved when a heterologous polynucleotide encoding an AIV H9-HA antigen is inserted in the same site as another heterologous polynucleotide, or when the expression of a heterologous polynucleotide encoding an AIV H9-HA antigen is linked to another heterologous polynucleotide, for example by an IRES or P2A sequence.

### Recombinant HVT Vectors

In a first aspect, the present invention provides a recombinant herpesvirus of turkeys (HVT) vector comprising a first heterologous polynucleotide encoding a first avian antigen and a second heterologous polynucleotide encoding a second avian antigen, further wherein the first and second heterologous polynucleotides are inserted in the same non-essential site in the recombinant HVT vector genome and wherein the first or second antigen is an avian influenza virus (AIV) subtype H9 haemagglutinin (AIV H9-HA) antigen. Preferably, AIV H9-HA is the second antigen. Herein, the avian antigen that is not the AIV H9-HA antigen, e.g. the avian antigen that is the first avian antigen when the AIV H9-HA antigen is the second antigen, may be referred to as the “additional avian antigen”. Preferably, the additional avian antigen is the first antigen as defined herein in respect of the present invention.

Herein, it will be understood that all references to an “avian antigen” refer to an antigen derived from an avian pathogen, that is, an antigen derived from a pathogen that is capable of infecting one or more avian species. Thus, it will be understood that an “avian antigen” as defined herein is not necessarily, e.g., an antigen derived from an avian itself. Accordingly, in an embodiment each avian antigen is an antigen derivable from an avian pathogen. In a preferred embodiment, each avian antigen is an antigen derivable from a different avian pathogen. In an embodiment, each avian antigen is not an antigen derivable from an avian cell, such as an uninfected, e.g. SPF, avian cell.

In an embodiment, the first heterologous polynucleotide is operably linked to a promoter, preferably to a strong promoter functional in eukaryotic cells. The promoters include, but are not limited to, an immediate early cytomegalovirus (CMV) promoter, guinea pig CMV promoter, an SV40 promoter, Pseudorabies Virus promoters such as that of glycoprotein X promoter, Herpes Simplex Virus-1 such as the alpha 4 promoter. Other promoters may include Marek's Disease Viruses (including MDV-1, MDV-2 and HVT) promoters, such as those driving glycoproteins gC, gB, gE, or gI expression, Infectious Laryngotracheitis Virus promoters such as those of glycoprotein gB, gE, gI, gD genes, or other herpesvirus promoters.

In embodiments, the recombinant vectors comprise a promoter. In embodiments, the promoter is selected from a murine cytomegalovirus (mCMV) promoter, a vaccinia virus H6 promoter, and a T7 promoter. In embodiments, the promoter is a CMV promoter. In embodiments, the promoter is a mCMV promoter. In embodiments, the promoter is a mCMV immediate early (IE) promoter. In embodiments, the first heterologous polynucleotide of the invention is operably linked to a promoter, and expression of the avian antigen encoded by the first heterologous polynucleotide is regulated by the promoter. In embodiments, the polynucleotide encoding the avian influenza H9-HA antigen and the additional heterologous polynucleotide encoding an additional avian antigen are operably linked to the same promoter, and their expression products are both regulated by the promoter. The AIV H9-HA antigen may be the first antigen or the second antigen. Preferably, AIV H9-HA is the second antigen. In embodiments, the polynucleotide encoding the avian influenza H9-HA antigen and the additional heterologous polynucleotide encoding an additional avian antigen are, independently, operably linked to different promoters, and their expression products are regulated by each respective promoter.

The present invention also encompasses the possibility that the order of the heterologous polynucleotides encoding for the antigens may be changed, that is the first heterologous polynucleotide encoding the first avian antigen may come second, and the second heterologous polynucleotide encoding the second avian antigen—that is, preferably the AIV-H9-HA antigen—may come first.

In a particularly preferred embodiment, the first heterologous polynucleotide is operably linked to a murine cytomegalovirus immediate early (mCMV IE) promoter. The term “operably linked” has the meaning normally attributed to it in the art, referring to the functional relationship between the promoter and the heterologous polynucleotide that results in the transcription of the heterologous polynucleotide, e.g. from DNA to RNA. Thus, in an embodiment, the transcription of the first heterologous polynucleotide is under the control of a promoter. Further, in a preferred embodiment, the transcription of the first heterologous polynucleotide is under the control of a mCMV IE promoter (also known as the mCMV IE1 promoter or the mCMV MIE promoter). In an embodiment, the first heterologous polynucleotide is transcribed from a promoter, preferably a mCMV IE promoter, preferably a mCMV IE1 promoter. Although the promoter is responsible for the transcription of the heterologous polynucleotide, the control of the promoter may also be defined relative to the transcription and translation process as a whole (protein production, or expression of the heterologous polynucleotide). Thus, in an embodiment, the expression of the first heterologous polynucleotide is under the control of a promoter, preferably a mCMV IE promoter, preferably a mCMV IE1 promoter. In a typical embodiment, the term “operably linked” means that the promoter is part of the same expression cassette comprising the promoter and the heterologous polynucleotide sequence. In a particularly preferred embodiment, the first and second heterologous polynucleotides are both operably linked to the same promoter. In a preferred form of this embodiment, the promoter is located 5′ of the first polynucleotide in the expression cassette, and the second polynucleotide is linked to the first polynucleotide by an IRES or P2A sequence. In a preferred form of this embodiment, the first polynucleotide and the IRES/P2A sequence are located between the second polynucleotide and the promoter in the expression cassette. In a preferred form of this embodiment, the promoter is located 5′ of the first polynucleotide in the expression cassette, the IRES/P2A is located 3′ of the first polynucleotide in the expression cassette, and the second polynucleotide is located 3′ of the IRES/P2A in the expression cassette.

In an embodiment, the first heterologous polynucleotide is operably linked to a promoter comprising a nucleotide sequence having at least 80% identity to SEQ ID NO: 8 or a fragment thereof and having equivalent activity thereto. In an embodiment, equivalent activity thereto means that the heterologous polynucleotide is transcribed at an equivalent level to the level of transcription when operably linked to the promoter of SEQ ID NO: 8. In an embodiment, equivalent activity thereto means that the heterologous polynucleotide is expressed at an equivalent level to the level of expression when operably linked to the promoter of SEQ ID NO: 8. In an embodiment wherein the first heterologous polynucleotide encodes an antigen of an avian pathogen, equivalent activity thereto means that the heterologous protection provided against that pathogen is at an equivalent level to the level of heterologous protection provided when operably linked to the promoter of SEQ ID NO: 8. In an embodiment in this context “equivalent” means functionally similar to, not substantially different from or the same as.

In an embodiment, the mCMV IE promoter comprises or consists of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 8 or a fragment thereof.

In an embodiment, the polynucleotide encoding the AIV H9-HA antigen comprises a polynucleotide sequence having at least 80% identity to SEQ ID NO: 9, and/or the AIV H9-HA antigen comprises an amino acid sequence having at least 80% identity to SEQ ID NO: 1. In an embodiment, the AIV H9-HA antigen is a Saudi Arabia strain H9-HA antigen. In an embodiment, the polynucleotide encoding the AIV H9-HA antigen is a Saudi Arabia strain H9-HA polynucleotide. In some embodiments, the avian influenza H9 polynucleotide is from strain A/avian/Saudi Arabia/910135/2006 (H9N2), and encodes a polypeptide defined by GenBank No: ACY80655.1, which is incorporated by reference herein in its entirety. In an embodiment, the polynucleotide encoding the AIV H9-HA antigen comprises a polynucleotide sequence having at least 80% identity to SEQ ID NO: 9 or a fragment thereof, and/or the AIV H9-HA antigen comprises an amino acid sequence having at least 80% identity to SEQ ID NO: 1 or a fragment thereof.

In an embodiment, the polynucleotide encoding the AIV H9-HA antigen comprises or consists of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 9 or a fragment thereof. In an embodiment, the polynucleotide encoding the AIV H9-HA antigen comprises or consists of a sequence that is codon-optimised for expression in an avian, preferably chicken, host having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 9 or a fragment thereof.

In an embodiment, the AIV H9-HA antigen comprises or consists of an amino acid sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 1 or a fragment thereof.

In an embodiment, the polynucleotide encoding the AIV H9-HA antigen is codon-optimised for expression in an avian host. Herein “codon-optimisation” has the meaning attributed to it in the art, and is a well-known general aspect of polynucleotides which is understood and readily practicable by the skilled person. Codon-optimisation involves the replacement of codons in a polynucleotide sequence with the equivalent codons (i.e. encoding the same amino acid) that are most favoured for expression in a particular host, such as an avian host, thereby providing a higher level of expression of the polynucleotide in that host. Codon-optimisation in respect of a polynucleotide also relates in clear structural and functional differences that can be observed relative to a non-codon-optimised version of the polynucleotide sequence, such that the skilled person can readily discriminate between the two. For example, the codon-optimisation of a heterologous polynucleotide sequence for expression in an avian host includes the replacement of codons with alternative codons that are more favoured for expression in an avian host, and therefore results in a different polynucleotide sequence. As a further example, the expression level of a polynucleotide that has been codon-optimised for expression in an avian host will be substantively and measurably higher than the expression level of the equivalent non-codon-optimised polynucleotide. Thus, in an embodiment, substantially all of, such as all of, the codons comprised in the polynucleotide encoding the AIV H9-HA antigen are the codons that are most favoured for expression in an avian host. In an embodiment, the expression of the polynucleotide encoding the AIV H9-HA antigen is substantially higher than the expression of the equivalent non-codon-optimised AIV H9-HA antigen in an avian host. Preferably, the host is a chicken. In embodiments, the recombinant vectors may comprise a sequence element that allows for translation initiation in a cap-independent manner, such as an internal ribosome entry site (IRES) or a sequence encoding a self-cleaving porcine teschovirus-1 2A or foot and mouth disease virus (FMDV) peptide (P2A). In a preferred embodiment, the element is an IRES sequence. In embodiments, the sequence element that allows for translation initiation in a cap-independent manner may be inserted in between two heterologous polynucleotides. In an embodiment, the sequence element is inserted in between the first heterologous polynucleotide encoding the first avian antigen and second heterologous polynucleotide encoding the AIV H9-HA antigen.

In an embodiment, the first heterologous polynucleotide is linked via an IRES or P2A sequence to the second heterologous polynucleotide. In an embodiment, “linked” means functionally linked. In an embodiment, transcription of the second heterologous polynucleotide is under the control of a promoter that is operably linked to the first polynucleotide, preferably a mCMV IE promoter. In an embodiment, transcription of the second heterologous polynucleotide is under the control of a promoter that is operably linked to the first polynucleotide, preferably a mCMV IE promoter, and translation of the second heterologous polynucleotide is under the control of (e.g. is initiated from) the IRES or P2A. In an embodiment, the recombinant HVT vector of the invention comprises, in the following order, the first heterologous polynucleotide sequence, the IRES and the second heterologous polynucleotide sequence. In an embodiment, the second heterologous polynucleotide is operably linked via the IRES or P2A to the promoter that is operably linked to the first heterologous polynucleotide. IRESs are known in the art and can be any internal sequence (e.g. between the first and second heterologous polynucleotide sequences) that permits ribosome entry (e.g. and the initiation of translation of the second heterologous polynucleotide). In an embodiment, the first heterologous polynucleotide is linked via an IRES or P2A to the second heterologous polynucleotide such that the first and second heterologous polynucleotides are transcribable or transcribed together, i.e. into the same (m) RNA molecule. Thus, in an embodiment, the transcription of the heterologous polynucleotides results in the production of a single RNA molecule comprising both the transcript of the first heterologous polynucleotide and the transcript of the second heterologous polynucleotide. In an embodiment, the first heterologous polynucleotide is linked via an IRES or P2A to the second heterologous polynucleotide such that both heterologous polynucleotides are expressible or expressed from the same molecule of RNA.

In an embodiment, the IRES comprises or consists of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 12 or a fragment thereof.

In an embodiment, the P2A comprises or consists of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 14 or a fragment thereof.

In an embodiment, the first avian antigen is different to the second avian antigen. In an embodiment, the first avian antigen is not an AIV H9-HA antigen. In an embodiment, the first avian antigen is derived from a pathogen that is not H9 type (e.g. H9N2) AIV. In an embodiment, the first avian antigen is derived from an avian pathogen other than AIV. In an embodiment, the first avian antigen is derived from infectious bursal disease virus (IBDV) or Newcastle disease virus (NDV). In an embodiment, the first avian antigen is an infectious bursal disease virus VP2 (IBDV VP2) antigen or a Newcastle disease virus F protein (NDV-F) antigen.

In an embodiment, the first avian antigen is derived from IBDV. In an embodiment, the first avian antigen is an IBDV VP2 antigen. In an embodiment, the IBDV VP2 antigen comprises an amino acid sequence having at least 80% identity to SEQ ID NO: 2. In an embodiment, the IBDV VP2 antigen comprises or consists of an amino acid sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 2 or a fragment thereof. In an embodiment, the polynucleotide encoding the IBDV VP2 antigen comprises or consists of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 10 or a fragment thereof.

In an embodiment, the first avian antigen is derived from NDV. In an embodiment, the first avian antigen is a NDV-F antigen. In an embodiment, the NDV-F antigen comprises an amino acid sequence having at least 80% identity to SEQ ID NO: 5. In an embodiment, the NDV F antigen is a NDV F antigen from NDV genotype VIId. In an embodiment, the NDV F antigen comprises or consists of an amino acid sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 5 or a fragment thereof. In an embodiment, the polynucleotide encoding the NDV F antigen comprises or consists of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 11 or a fragment thereof.

In an embodiment, the recombinant HVT vector further comprises a polyadenylation signal. In a preferred embodiment, the recombinant HVT vector further comprises an SV40 polyadenylation signal. In an embodiment, the (e.g. SV40) polyadenylation signal is located 3′ of the second heterologous polynucleotide, relative to the expression direction of the second heterologous polynucleotide. In an embodiment, the (e.g. SV40) polyadenylation signal is operably linked to the second heterologous polynucleotide. In an embodiment, the polyadenylation signal being operably linked means that the polyadenylation signal is functionally linked to the second heterologous polynucleotide. In embodiments, the polyA signal is a simian virus 40 (SV40) polyA tail. In embodiments, expression of the avian influenza H9 antigen is influenced by the polyA signal. In embodiments, the polyA signal is located downstream of the polynucleotide encoding the avian influenza H9 antigen. In embodiments, polyA signal is located downstream of the first heterologous polynucleotide encoding the first antigen. In embodiments, expression of the avian antigen encoded by the first heterologous polynucleotide is influenced by the polyA signal. In embodiments, the recombinant vectors do not comprise a polyA signal which influences expression of the avian influenza H9 antigen and/or the first avian antigen.

In an embodiment, the recombinant HVT vector comprises an expression cassette with the following elements:
(a) the first heterologous polynucleotide; and
(b) the second heterologous polynucleotide;
   wherein one of the heterologous polynucleotides encodes an AIV H9-HA antigen and the other heterologous polynucleotide encodes an additional avian antigen.

In an embodiment, the recombinant HVT vector comprises an expression cassette with the following elements:
(a) optionally a promoter, preferably a mCMV IE promoter;
(b) the first heterologous polynucleotide;
(c) optionally an IRES or P2A sequence;
(d) the second heterologous polynucleotide; and
(e) optionally an SV40 polyadenylation signal;
   wherein one of the heterologous polynucleotides encodes an AIV H9-HA antigen and the other heterologous polynucleotide encodes a an additional avian antigen.

In an embodiment, the recombinant HVT vector comprises an expression cassette with the following elements:
(a) optionally a promoter, preferably a mCMV IE promoter;
(b) the first heterologous polynucleotide;
(c) an IRES or P2A sequence;
(d) the second heterologous polynucleotide; and
(e) optionally an SV40 polyadenylation signal;
   wherein one of the heterologous polynucleotides encodes an AIV H9-HA antigen and the other heterologous polynucleotide encodes an additional avian antigen.

In an embodiment, the recombinant HVT vector comprises an expression cassette with the following elements:
(a) a promoter, preferably a mCMV IE promoter;
(b) the first heterologous polynucleotide;
(c) an IRES or P2A sequence;
(d) the second heterologous polynucleotide; and
(e) optionally an SV40 polyadenylation signal;
   wherein one of the heterologous polynucleotides encodes an AIV H9-HA antigen and the other heterologous polynucleotide encodes an additional avian antigen.

In an embodiment, the recombinant HVT vector comprises an expression cassette with the following elements:
(a) a mCMV IE promoter;
(b) the first heterologous polynucleotide;
(c) the second heterologous polynucleotide; and
(d) an SV40 polyadenylation signal;
   wherein one of the heterologous polynucleotides encodes an AIV H9-HA antigen and the other heterologous polynucleotide encodes an additional avian antigen.

In an embodiment, the recombinant HVT vector comprises an expression cassette which further comprises an IRES or P2A sequence between the first and the second heterologous polynucleotide.

In an embodiment, the recombinant HVT vector comprises an expression cassette with the following elements:
(a) a mCMV IE promoter;
(b) the first heterologous polynucleotide;
(c) an IRES sequence;
(d) the second heterologous polynucleotide; and
(e) an SV40 polyadenylation signal;
   wherein one of the heterologous polynucleotides encodes an AIV H9-HA antigen and the other heterologous polynucleotide encodes an additional avian antigen.

In an embodiment, the recombinant HVT vector comprises an expression cassette with the following elements:
(a) a mCMV IE promoter;
(b) the first heterologous polynucleotide;
(c) a P2A sequence;
(d) the second heterologous polynucleotide; and
(e) an SV40 polyadenylation signal;
   wherein one of the heterologous polynucleotides encodes an AIV H9-HA antigen and the other heterologous polynucleotide encodes an additional avian antigen.

In an embodiment, the recombinant HVT vector comprises an expression cassette with the following elements in the following order:
(a) the first heterologous polynucleotide; and
(b) the second heterologous polynucleotide;
   wherein the second heterologous polynucleotide encodes an AIV H9-HA antigen and the first heterologous polynucleotide encodes an additional avian antigen.

In an embodiment, the recombinant HVT vector comprises an expression cassette with the following elements in the following order:
(a) optionally a promoter, preferably a mCMV IE promoter;
(b) the first heterologous polynucleotide;
(c) optionally an IRES or P2A sequence;
(d) the second heterologous polynucleotide; and
(e) optionally an SV40 polyadenylation signal;
   wherein the second heterologous polynucleotide encodes an AIV H9-HA antigen and the first heterologous polynucleotide encodes an additional avian antigen.

In an embodiment, the recombinant HVT vector comprises an expression cassette with the following elements in the following order:
(a) optionally a promoter, preferably a mCMV IE promoter;
(b) the first heterologous polynucleotide;
(c) an IRES or P2A sequence;
(d) the second heterologous polynucleotide; and
(e) optionally an SV40 polyadenylation signal;
   wherein the second heterologous polynucleotide encodes an AIV H9-HA antigen and the first heterologous polynucleotide encodes an additional avian antigen.

In an embodiment, the recombinant HVT vector comprises an expression cassette with the following elements in the following order:
(a) a promoter, preferably a mCMV IE promoter;
(b) the first heterologous polynucleotide;
(c) an IRES or P2A sequence;
(d) the second heterologous polynucleotide; and
(e) optionally an SV40 polyadenylation signal;
   wherein the second heterologous polynucleotide encodes an AIV H9-HA antigen and the first heterologous polynucleotide encodes an additional avian antigen.

In an embodiment, the recombinant HVT vector comprises an expression cassette with the following elements in the following order:
(a) a mCMV IE promoter;
(b) the first heterologous polynucleotide;
(c) the second heterologous polynucleotide; and
(d) an SV40 polyadenylation signal;
   wherein the second heterologous polynucleotide encodes an AIV H9-HA antigen and the first heterologous polynucleotide encodes an additional avian antigen.

In an embodiment, the recombinant HVT vector comprises an expression cassette which further comprises an IRES or P2A sequence between the first and the second heterologous polynucleotide.

In an embodiment, the recombinant HVT vector comprises an expression cassette with the following elements in the following order:
(a) a mCMV IE promoter;
(b) the first heterologous polynucleotide;
(c) an IRES sequence;
(d) the second heterologous polynucleotide; and
(e) an SV40 polyadenylation signal;
   wherein the second heterologous polynucleotide encodes an AIV H9-HA antigen and the first heterologous polynucleotide encodes an additional avian antigen.

In an embodiment, the recombinant HVT vector comprises an expression cassette with the following elements in the following order:
(a) a mCMV IE promoter;
(b) the first heterologous polynucleotide;
(c) a P2A sequence;
(d) the second heterologous polynucleotide; and
(e) an SV40 polyadenylation signal;
   wherein the second heterologous polynucleotide encodes an AIV H9-HA antigen and the first heterologous polynucleotide encodes an additional avian antigen.

It is to be understood herein that the “additional” or “further” avian antigen may be either an IBDV antigen, preferably an IBDV VP2 antigen, or an NDV antigen, preferably an NDV-F antigen.

In such embodiments and in general when referring to the recombinant elements of the recombinant HVT vector or the expression cassette therein (e.g. the expression cassette of the present invention), the “order” refers to the relative order of the recombinant elements alone. Thus, in an embodiment, the order is 5′ to 3′ within the expression cassette, from the first recombinant element (i.e. heterologous or non-HVT element), such as the mCMV IE promoter, to the last recombinant element, such as the SV40 polyadenylation signal, that is specified. In an embodiment, the order of the recombinant elements follows the direction of transcription of the first heterologous polynucleotide.

In another respect, it is to be understood herein that the “order” of the recombinant elements does not necessarily relate to the 5′ to 3′ direction of the (+) sense strand of the HVT vector genome. It is to be understood that the recombinant elements/expression cassette of the recombinant HVT vector of the present invention can be inserted in either direction into the HVT genome. Thus, in an embodiment, the recombinant elements are inserted 5′ to 3′ relative to the sense strand of the HVT vector genome, and 3′ to 5′ relative to the antisense strand of the HVT vector genome. In an embodiment, the recombinant elements are inserted 5′ to 3′ relative to the antisense strand of the HVT vector genome, and 3′ to 5′ relative to the sense strand of the HVT vector genome. In an embodiment exemplified herein, the recombinant HVT vector comprises the recombinant elements 5′ to 3′ relative to the antisense strand of the recombinant HVT vector genome.

Accordingly, in an embodiment the “order” of the elements is the order of the elements 5′ to 3′ within the expression cassette, e.g. the sense strand of the expression cassette. Accordingly, in an embodiment the “order” of the elements is the order of the elements 5′ to 3′ within the antisense strand of the recombinant HVT vector genome. In such embodiments, the antisense strand of the recombinant HVT vector genome may be defined as the strand from which the genes flanking the expression cassette, e.g. the endogenous HVT genes flanking the expression cassette, are not expressed. Accordingly, in an embodiment the “order” of the elements is the order of the elements starting from the direction of transcription of the first heterologous polynucleotide. In such embodiments, the transcription may be defined as that which is capable of expressing the first avian antigen.

Herein, it is to be understood that unless otherwise specified each reference to a heterologous polynucleotide, polynucleotide sequence, nucleotide sequence, nucleic acid or equivalent term encompasses not only the nucleotide sequence per se, but also the complement of that nucleotide sequence, the reverse of that nucleotide sequence, and the reverse complement of that nucleotide sequence. Such references further encompass the RNA equivalent of that nucleotide sequence, the RNA complement (or transcript) of that nucleotide sequence, the reverse of the RNA equivalent of that nucleotide sequence and the reverse of the RNA complement (or transcript) of that nucleotide sequence. Unless otherwise indicated, such references further encompass any modifications or routine or non-substantive variations to any of those sequences, which are well-known in the art and further described herein.

The term “non-essential” site is well-known in the art of recombinant HVT vectors, of which many specific examples are known in the art. In an embodiment, a non-essential site includes any locus in the HVT vector genome that is not within an expressed sequence (open reading frame, ORF) that is required for viral replication. In an embodiment, a non-essential site is not within an ORF. However, it will be understood by the skilled person that in an embodiment a non-essential site may be within an ORF that is not required for viral replication, such as (without wishing to be bound by theory) within the US10, SORF3 or US2 ORFs of the unique short (Us) region of the HVT vector genome. In an embodiment, the non-essential site is selected from any of the UL44 (gC) locus, the site located between UL44 and UL45 ORFs, the site located between UL45 and UL46 ORFs, the IG1 locus, the IG2 locus, the US2 locus, the site located between US2 and SORF3 ORFs, the SORF3 locus, the site located between SORF3 and US10 ORFs, or the US10 locus. In a preferred embodiment, the insertion site is in an intergenic region of the HVT vector genome.

In a particularly preferred embodiment of the present invention, the insertion site is an IG1 site. In an embodiment, the site is in the intergenic region 1 of the HVT vector genome. In an embodiment, the insertion site is in an IG1 locus of the HVT vector genome. In an embodiment, the IG1 locus is defined as the region of the HVT vector genome between the ATG of ORF UL55 and the junction of UL (the unique long region) with the adjacent repeat region (as described in U.S. Pat. No. 5,980,906).

Herein, the first and second polynucleotides being inserted into the “same” nonessential site has the meaning as would be understood by the skilled person, i.e. that the first and second polynucleotides are inserted into the same defined insertion site locus or region, e.g. that both polynucleotides are inserted into an IG1 site of the HVT vector genome. The skilled person well understands and is able to take account of the fact that the insertion of heterologous sequences may result in the insertion or deletion of one or more HVT vector genome or non-HVT vector genome nucleotides, which are non-significant variations encompassed by the present invention. In an embodiment, the first and second polynucleotide sequences are inserted into the same insertion site in the sense that the second polynucleotide sequence is operably linked to the same promoter as the first polynucleotide sequence, such as via an IRES or P2A sequence. In an embodiment, the first and second polynucleotide sequences are inserted into the same insertion site in the sense that the second polynucleotide sequence is linked to the first polynucleotide sequence, such as via an IRES or P2A sequence. In an embodiment, the first and second polynucleotide sequences are inserted into the same insertion site in the sense that there is substantively no HVT vector genomic DNA between the first and second heterologous polynucleotide sequences.

In an embodiment, the recombinant HVT vector is capable of stably expressing the first and second avian antigens (e.g. the additional antigen and AIV H9-Ha antigen respectively). In an embodiment, “stably expressing” means that both avian antigens are still expressed even after passaging, such as cellular passaging in vitro. In an embodiment, “stably expressing” means that both avian antigens are still expressed after several passages, for example at least 6, at least 7, at least 8, at least 9, at least 10, at least 11, at least 12, at least 13, at least 14 or at least 15 passages. In an embodiment, the recombinant HVT vector is stable for at least 6, at least 7, at least 8, at least 9, at least 10, at least 11, at least 12, at least 13, at least 14 or at least 15 passages cellular passages in vitro.

In an embodiment, “stably expressing” means that the level of expression of both avian antigens is essentially unchanged. In an embodiment, “stably expressing” means that the level of expression of both avian antigens is essentially the same as the level of expression prior to (any) passaging. In an embodiment, “stably expressing” means that the level of expression of both avian antigens is capable of inducing heterologous protection in vivo (e.g. in an avian, such as a chicken) against the avian pathogens from which the antigens are derived.

In an embodiment, the recombinant HVT vector is capable of inducing a protective immune response against AIV and the pathogen from which the additional (e.g. the first) avian antigen is derived. In an embodiment, the pathogen from which the additional (e.g. the first) avian antigen is derived is NDV. In an embodiment, the pathogen from which the additional (e.g. the first) avian antigen is derived is IBDV. As has been mentioned herein throughout, the additional avian antigen is preferably the first avian antigen encoded by the first heterologous polynucleotide as defined herein.

The recombinant HVT vector of the invention is useful as a vaccine against both MDV and AIV, which can be administered to an avian to induce protection against MDV (via the presence of the HVT vector genome) and heterologous protection against AIV. In an embodiment of the recombinant HVT vector of the invention, the recombinant HVT vector is capable of inducing a protective immune response against AIV. In an embodiment, the recombinant HVT vector is capable of inducing a protective immune response against an additional heterologous pathogen. In an embodiment, the recombinant HVT vector is capable of inducing a protective immune response against NDV. In an embodiment, the recombinant HVT vector is capable of inducing a protective immune response against IBDV. In an embodiment, the recombinant HVT vector is capable of inducing a protective immune response against AIV and also NDV or IBDV. In an embodiment, the recombinant HVT vector is capable of inducing a protective immune response against MDV, AIV and also NDV or IBDV. In an embodiment, the protective immune response comprises a reduction of clinical signs caused by MDV and/or AIV and/or IBDV or NDV. In an embodiment, the clinical signs are respiratory signs. In an embodiment, the protective immune response comprises reducing shedding caused by AIV in an avian. In an embodiment, the recombinant HVT vector of the invention is capable of reducing shedding caused by AIV in an avian. In an embodiment, the recombinant HVT vector of the invention is capable of reducing shedding caused by AIV in an avian following AIV infection or challenge. In an embodiment, the protective immune response comprises reducing clinical signs caused by MDV and/or AIV and/or IBDV or NDV as well as reducing shedding caused by AIV. In an embodiment, the reduction is relative to the clinical signs of an equivalent unvaccinated avian, such as an avian that has not received an MDV and/or AIV and/or IBDV or NDV vaccine and has been infected or challenged with NDV and/or AIV and/or IBDV or NDV.

Herein, in an embodiment it is understood that “MDV and/or AIV and/or IBDV or NDV” refers to the recombinant HVT vector providing protection etc. against MDV due to the presence of the HVT vector genome and heterologous protection against AIV due to the presence of the AIV antigen (i.e. AIV H9-HA), as well as heterologous protection against an additional avian pathogen from which the antigen encoded by the additional (e.g. the first) heterologous polynucleotide is derived (e.g. which may be IBDV or NDV). Thus, herein “MDV and/or AIV and/or IBDV or NDV” is interchangeable with “MDV and/or AIV and/or the avian pathogen from which the antigen encoded by the additional (e.g. the first) heterologous polynucleotide is derived”.

In an embodiment of the recombinant HVT vector of the invention, one dose of the recombinant HVT vector is capable of inducing a protective immune response against MDV and/or AIV and/or NDV or IBDV. In an embodiment, this means that a protective immune response against MDV and/or AIV and/or NDV or IBDV will be induced in an avian that has received (only) a single administration of the recombinant HVT vector of the invention. In an embodiment, this means that a protective immune response against MDV and/or AIV and/or NDV or IBDV will be induced in an avian that has received (only) a single administration of any MDV and/or AIV and/or NDV or IBDV vaccine or antigen, wherein the single administration is of the recombinant HVT vector of the invention. Thus, it will be understood that in an embodiment one dose alone of the recombinant vector is capable of inducing a protective immune response against MDV and/or AIV and/or NDV or IBDV. In an embodiment, it will be understood that the protective immune response is induced when (only) a single dose of the recombinant HVT vector of the invention has been administered. In an embodiment, “one dose” means that a second or further dose of the recombinant HVT vector of the invention is not necessary for the induction of the protective immune response. In an embodiment, “one dose” means that a second or further dose of any MDV and/or AIV and/or NDV or IBDV vaccine or antigen is not necessary for the induction of the protective immune response. In an embodiment “one dose” means that the avian has not been primed against the same avian pathogen(s) to which the invention relates. For example, the avian has not been primed with the same vaccine, vector or antigen to which the invention relates, or any other type of antigen (e.g. killed vaccine, subunit vaccine, etc). In an embodiment, the one dose is about 6.0 log 10 EID50 or 2500 PFU or 3.4 log 10 PFU.

In an embodiment of the recombinant HVT vector of the invention, the recombinant HVT vector is capable of inducing a protective immune response against MDV and/or AIV and/or NDV or IBDV when administered to an avian of six or fewer days of age, five or fewer days of age, four or fewer days of age, three or fewer days of age, two or fewer days of age, one or fewer day of age, or one day of age. In an embodiment, the recombinant HVT vector of the invention will be capable of inducing a protective immune response against MDV and/or AIV and/or NDV or IBDV even when administered to an avian of one day of age.

In an embodiment, the recombinant HVT vector of the invention is capable of inducing or induces a protective immune response against IBDV at 3 weeks post vaccination. In an embodiment, the recombinant HVT vector of the invention is capable of inducing or induces a protective immune response against IBDV at 4 weeks post vaccination. In preferred embodiments of this type, the recombinant HVT vector comprises a first heterologous polynucleotide encoding an IBDV antigen, preferably an IBDV VP2 antigen.

In an embodiment, the recombinant HVT vector of the invention is capable of inducing or induces complete protection against NDV. In an embodiment, complete protection means that subsequent NDV challenge or infection does not induce any clinical signs. In an embodiment, the recombinant HVT vector of the invention is capable of inducing or induces 100% protection against NDV challenge or infection at 21 days after vaccination. In an embodiment, the recombinant HVT vector of the invention is capable of inducing or induces 100% protection against NDV challenge or infection with the MALAYSIAN MAL04-01 strain at 21 days after vaccination. In preferred embodiments of this type, the recombinant HVT vector comprises a first heterologous polynucleotide encoding a NDV antigen, preferably a NDV F antigen.

In an embodiment, the recombinant HVT vector of the invention is vHVT513. In an embodiment, vHVT513 is defined as a recombinant HVT vector comprising a first heterologous polynucleotide encoding an IBDV VP2 antigen and a second heterologous polynucleotide encoding an AIV H9-HA antigen, wherein the first heterologous polynucleotide is operably linked to a mCMV IE promoter, the second heterologous polynucleotide is linked by an IRES to the first heterologous polynucleotide, and the first and second heterologous polynucleotides are both comprised in the same site in the HVT vector genome which is an IG1 site. In an embodiment, vHVT513 is defined as a recombinant HVT vector comprising a recombinant sequence comprising or consisting of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 3 or a fragment thereof. In an embodiment, vHVT513 is defined as a recombinant HVT vector comprising or consisting of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 4 or a fragment thereof.

In an embodiment, the recombinant HVT vector of the invention is vHVT514. In an embodiment, vHVT514 is defined as a recombinant HVT vector comprising a first heterologous polynucleotide encoding a NDV F antigen and a second heterologous polynucleotide encoding an AIV H9-HA antigen, wherein the first heterologous polynucleotide is operably linked to a mCMV IE promoter, the second heterologous polynucleotide is linked by an IRES to the first heterologous polynucleotide, and the first and second heterologous polynucleotides are both comprised in the same site in the HVT vector genome which is an IG1 site. In an embodiment, vHVT514 is defined as a recombinant HVT vector comprising a recombinant sequence comprising or consisting of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 6 or a fragment thereof. In an embodiment, vHVT514 is defined as a recombinant HVT vector comprising or consisting of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 7 or a fragment thereof.

In an embodiment, the SV40 polyadenylation signal referred to herein comprises or consists of a sequence having 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 13 or a fragment thereof.

The recombinant vectors of the invention comprise a polynucleotide encoding an additional avian antigen from a pathogenic organism other than avian influenza. In embodiments, the additional polynucleotide encodes an NDV antigen or an Infectious Bursal Disease Virus (IBDV) antigen. In embodiments, the recombinant vectors comprise a heterologous polynucleotide encoding a NDV fusion protein (NDV-F). In embodiments, the recombinant vectors comprise a heterologous polynucleotide encoding an IBDV VP2 capsid protein (IBDV VP2).

In embodiments, the polynucleotide encoding the NDV-F antigen comprises at least 75%, 80%, 85%, 90%, 95%, 96%, 97%, 98%, 99%, or 100% sequence identity to the polynucleotide sequence of SEQ ID NO: 11 which encodes for the NDV-F protein, or any fragments thereof. In embodiments, the polynucleotide encoding the NDV-F antigen encodes an NDV-F polypeptide comprising at least 75%, 80%, 85%, 90%, 95%, 96%, 97%, 98%, 99%, or 100% sequence identity to an amino acid sequence as set forth in SEQ ID NO: 5, or any fragments thereof.

In embodiments, the polynucleotide encoding the IBDV VP2 antigen comprises at least 75%, 80%, 85%, 90%, 95%, 96%, 97%, 98%, 99%, or 100% sequence identity to the polynucleotide sequence of SEQ ID NO: 10 which encodes for the IBDV VP2 capsid protein, or any fragments thereof. In embodiments, the polynucleotide encoding the IBDV VP2 antigen encodes an IBDV VP2 polypeptide comprising at least 75%, 80%, 85%, 90%, 95%, 96%, 97%, 98%, 99%, or 100% sequence identity to a polypeptide sequence as set forth in SEQ ID NO: 2, or any fragments thereof.

In embodiments, the recombinant vectors comprise a HVT backbone vector. In embodiments, the backbone vector is an HVT FC126 strain (Igarashi, T., et al. “Restriction enzyme map of herpesvirus of turkey DNA and its collinear relationship with Marek's disease virus DNA.” Virology 157.2 (1987): 351-358).

### Further Specific Forms

In a second aspect, the present invention provides a nucleic acid molecule comprising the recombinant HVT vector of the invention.

In a third aspect, the present invention provides an antigen expression cassette comprising the following elements:
(a) optionally a mCMV IE promoter;
(b) a first heterologous polynucleotide encoding a first avian antigen;
(c) optionally an IRES or P2A sequence;
(d) a second heterologous polynucleotide encoding a second avian antigen; and
(e) optionally an SV40 polyadenylation signal;
   wherein the first or second avian antigen is an AIV antigen, preferably an AIV H9-HA antigen.

In an embodiment, the present invention provides an antigen expression cassette comprising the following elements:
(a) optionally a mCMV IE promoter;
(b) a first heterologous polynucleotide encoding a first avian antigen;
(c) optionally an IRES or P2A sequence;
(d) a second heterologous polynucleotide encoding a second avian antigen that is an AIV antigen, preferably an AIV H9-HA antigen; and
(e) optionally an SV40 polyadenylation signal.

In an embodiment, the present invention provides an antigen expression cassette comprising the following elements:
(a) a mCMV IE promoter;
(b) a first heterologous polynucleotide encoding a first avian antigen;
(c) optionally an IRES or P2A sequence;
(d) a second heterologous polynucleotide encoding a second avian antigen that is an AIV antigen, preferably an AIV H9-HA antigen; and
(e) optionally an SV40 polyadenylation signal.

In an embodiment, the present invention provides an antigen expression cassette comprising the following elements:
(a) a mCMV IE promoter;
(b) a first heterologous polynucleotide encoding a first avian antigen;
(c) optionally an IRES or P2A sequence;
(d) a second heterologous polynucleotide encoding a second avian antigen that is an AIV antigen, preferably an AIV H9-HA antigen; and
(e) an SV40 polyadenylation signal.

Alternatively or additionally in a third aspect, the present invention provides an antigen expression cassette comprising in the following order 5′ to 3′:
(a) optionally a mCMV IE promoter;
(b) a first heterologous polynucleotide encoding a first avian antigen;
(c) optionally an IRES or P2A sequence;
(d) a second heterologous polynucleotide encoding a second avian antigen that is an AIV antigen, preferably an AIV H9-HA antigen; and
(e) optionally an SV40 polyadenylation signal.

In an embodiment, the present invention provides an antigen expression cassette comprising in the following order 5′ to 3′:
(a) a mCMV IE promoter;
(b) a first heterologous polynucleotide encoding a first avian antigen;
(c) optionally an IRES or P2A sequence;
(d) a second heterologous polynucleotide encoding a second avian antigen that is an AIV antigen, preferably an AIV H9-HA antigen; and
(e) optionally an SV40 polyadenylation signal.

In an embodiment, the present invention provides an antigen expression cassette comprising in the following order 5′ to 3′:
(a) a mCMV IE promoter;
(b) a first heterologous polynucleotide encoding a first avian antigen;
(c) optionally an IRES or P2A sequence;
(d) a second heterologous polynucleotide encoding a second avian antigen that is an AIV antigen, preferably an AIV H9-HA antigen; and
(e) an SV40 polyadenylation signal.

In an embodiment, the present invention provides an antigen expression cassette comprising in the following order 5′ to 3′:
(a) a mCMV IE promoter;
(b) a first heterologous polynucleotide encoding a first avian antigen;
(c) an IRES sequence;
(d) a second heterologous polynucleotide encoding a second avian antigen that is an AIV antigen, preferably an AIV H9-HA antigen; and
(e) an SV40 polyadenylation signal.

In an embodiment, the present invention provides an antigen expression cassette comprising in the following order 5′ to 3′:
(a) a mCMV IE promoter;
(b) a first heterologous polynucleotide encoding a first avian antigen;
(c) a P2A sequence;
(d) a second heterologous polynucleotide encoding a second avian antigen that is an AIV antigen, preferably an AIV H9-HA antigen; and
(e) an SV40 polyadenylation signal.

As has been mentioned herein, the “order” 5′ to 3′ relates to the order of the recombinant elements in the expression cassette, and is not necessarily relative to the 5′ to 3′ direction of the HVT vector genome sense strand. In an embodiment, the cassette is capable of expressing two avian antigens. In an embodiment, the cassette is solely capable of expressing two avian antigens. It is to be understood herein that the two avian antigens are that encoded by the first heterologous polynucleotide (e.g. an IBDV VP2 or NDV F antigen) and that encoded by the second heterologous polynucleotide (e.g. an AIV antigen or an AIV H9-HA antigen).

In a fourth aspect, the present invention provides a cell comprising the recombinant HVT vector, the nucleic acid molecule or the expression cassette of the invention.

### Compositions

In a fifth aspect, the present invention provides a composition comprising the recombinant HVT vector, the nucleic acid molecule, the expression cassette or the cell of the invention.

The present invention also provides immunogenic compositions and vaccines comprising any of the recombinant vectors described herein. In embodiments, the recombinant viral vectors of the present invention can be used in immunogenic compositions and vaccines to elicit an immune response against, and/or provide animals with protection against MDV and/or AIV and/or IBDV or NDV.

In some embodiments, the immunogenic compositions are effective to elicit, induce, and/or stimulate an immune response in an animal, such as an avian, when administered to the animal. In embodiments, the immunogenic compositions of the present invention are formulated such that they are safe and effective to elicit immunity against MDV and/or AIV and/or IBDV or NDV when administered to an animal.

In some embodiments, the vaccines are effective to elicit, induce, and/or stimulate a protective immune response in an animal, such as an avian, when administered to the animal. In embodiments, the vaccines of the present invention are formulated such that they are safe and effective to elicit protective immunity against MDV and/or AIV and/or IBDV or NDV when administered to an animal.

In embodiments, the immunogenic compositions and vaccines comprise a pharmaceutically or veterinarily acceptable carrier, excipient, and/or adjuvant. Examples of suitable pharmaceutically or veterinarily acceptable carriers include 0.9% NaCl (e.g., saline) solution, phosphate buffer, poly-(L-glutamate), lactated Ringer's injection diluent (sodium chloride, sodium lactate, potassium chloride and calcium chloride), and polyvinylpyrrolidone. Examples of pharmaceutically or veterinarily acceptable adjuvants include, (1) polymers of acrylic or methacrylic acid, maleic anhydride and alkenyl derivative polymers, (2) immunostimulating sequences (ISS), such as oligodeoxyribonucleotide sequences having one or more non-methylated CpG units, (3) an oil in water emulsion, (4) cation lipids containing a quaternary ammonium salt, e.g., DDA (5) cytokines, (6) aluminum hydroxide or aluminum phosphate, and/or (7) saponin.

In embodiments, the immunogenic compositions and vaccines are formulated for administration to an animal, such as an avian. In embodiments, the immunogenic compositions and vaccines are formulated for administration by one or more of the following routes: aerosol (spray), ocular, nasal, oculonasal, oral, subcutaneous injection, and/or intramuscular injection. In a preferred embodiment, the immunogenic compositions and vaccines are formulated for administration by subcutaneous injection.

In embodiments, the immunogenic compositions and vaccines can contain a titer of recombinant vector from 10² to 10³, 10³ to 10⁴, 10⁴ to 10⁵, 10⁵ to 10⁶, 10⁶ to 10⁷, 10⁷ to 10⁸, 10⁸ to 10⁹, 10⁹ to 10¹⁰, 10³ to 10⁸, 10⁴ to 10⁸, or 10⁵ to 10⁷, per dose. The recombinant vector may be titrated based on any virus titration methods including, but not limited to, FFA (Focus Forming Assay) or FFU (Focus Forming Unit), TCID₅₀ (50% Tissue Culture Infective Dose), EID₅₀ (50% Egg Infective Dose), PFU (Plaque Forming Units), and FAID₅₀ (50% Fluorescent Antibody Infectious Dose). In embodiments, the dose volumes can be between 0.01 and 10 ml, between 0.01 and 5 ml, between 0.01 and 1 ml, or 0.01 and 0.5 ml.

### Methods, Applications and Uses

In a sixth aspect, the present invention provides the recombinant HVT vector, the nucleic acid molecule, the expression cassette, the cell or the composition of the invention, for use in a method of inducing a protective immune response against AIV, the pathogen from which the additional (e.g. the first) avian antigen is derived and/or MDV in an avian.

Alternatively in a sixth aspect, the present invention provides the recombinant HVT vector, the nucleic acid molecule, the cell or the composition of the invention, for use in a method of inducing a protective immune response against AIV, the pathogen from which the additional (e.g. the first) avian antigen is derived and/or MDV in an avian.

In a seventh aspect, the present invention provides a method of inducing a protective immune response against AIV, the pathogen from which the additional (e.g. the first) avian antigen is derived and/or MDV in an avian, comprising administering the recombinant HVT vector, the nucleic acid molecule, the expression cassette, the cell or the composition of the invention to the avian.

Alternatively in a seventh aspect, the present invention provides a method of inducing a protective immune response against AIV, the pathogen from which the additional (e.g. the first) avian antigen is derived and/or MDV in an avian, comprising administering the recombinant HVT vector, the nucleic acid molecule, the cell or the composition of the invention to the avian.

In an eighth aspect, the present invention provides the recombinant HVT vector, the nucleic acid molecule, the expression cassette, the cell or the composition of the invention, for use in the manufacture of a medicament for inducing a protective immune response against AIV, the pathogen from which the additional (e.g. the first) avian antigen is derived and/or MDV in an avian.

Alternatively in an eighth aspect, the present invention provides the recombinant HVT vector, the nucleic acid molecule, the cell or the composition of the invention, for use in the manufacture of a medicament for inducing a protective immune response against AIV, the pathogen from which the additional (e.g. the first) avian antigen is derived and/or MDV in an avian.

In an embodiment, inducing a protective immune response comprises reducing clinical signs associated with infection by AIV, the pathogen from which the additional (e.g. the first) avian antigen is derived and/or MDV in an avian. In an embodiment, the clinical signs are respiratory clinical signs.

In a ninth aspect, the present invention provides the recombinant HVT vector, the nucleic acid molecule, the expression cassette, the cell or the composition of the invention, for use in a method of reducing AIV shedding in an avian.

Alternatively in a ninth aspect, the present invention provides the recombinant HVT vector, the nucleic acid molecule, the cell or the composition of the invention, for use in a method of reducing AIV shedding in an avian.

In a tenth aspect, the present invention provides a method of reducing AIV shedding in an avian, comprising administering the recombinant HVT vector, the nucleic acid molecule, the expression cassette, the cell or the composition of the invention to the avian.

Alternatively in a tenth aspect, the present invention provides a method of reducing AIV shedding in an avian, comprising administering the recombinant HVT vector, the nucleic acid molecule, the cell or the composition of the invention to the avian.

In an eleventh aspect, the present invention provides the recombinant HVT vector, the nucleic acid molecule, the expression cassette, the cell or the composition of the invention, for use in the manufacture of a medicament for reducing AIV shedding in an avian.

Alternatively in an eleventh aspect, the present invention provides the recombinant HVT vector, the nucleic acid molecule, the cell or the composition of the invention, for use in the manufacture of a medicament for reducing AIV shedding in an avian.

In an embodiment, the recombinant HVT vector is administered once. In an embodiment, this means that the recombinant HVT vector is only administered once. In an embodiment, this means that second/further administrations of the recombinant HVT vector are not carried out. In an embodiment, the recombinant HVT vector is administered once and the protective immune response is obtained after such single administration. In another embodiment, this means that the initial administration of the recombinant HVT vector only comprises one dose to induce a protective immune response, but the administration of one or more booster administrations comprising further doses at a substantially later date are not excluded. In an embodiment, the one dose is about 6.0 log 10 EID₅₀ or 2500 PFU or 3.4 log 10 PFU.

In an embodiment, the recombinant HVT vector is administered to an avian at six or fewer days of age, five or fewer days of age four or fewer days of age, three or fewer days of age, two or fewer days of age, one or 1 fewer days of age, or one day of age. In an embodiment, the recombinant HVT vector of the invention is administered to an avian of one day of age and induces a protective immune response against MDV and/or AIV and/or IBDV or NDV.

In a preferred embodiment, the recombinant HVT vector is administered subcutaneously.

In an embodiment, the recombinant HVT vector is administered to an avian, such as a chick, that is positive for passively acquired AIV H9-HA antibodies. In an embodiment, the passively acquired AIV H9-HA antibodies are maternally derived antibodies (MDAs). In an embodiment, the recombinant HVT vector is capable of inducing a protective immune response against AIV subtype H9 in an avian that is positive for passively acquired AIV H9-HA antibodies, such as AIV H9-HA MDAs. In one embodiment, substantially no immunological interference is evident between the passively acquired AIV H9 HA antibodies in the avian and the protective immune response elicited by the recombinant HVT vector of the invention. In one embodiment, the mean H9-HA MDA level at the day of vaccination DO is at least 2.00 log 10, 2.50 log 10, 2.75 log 10, 3.00 log 10, 3.25 log 10, 3.50 log 10, 3.75 log 10 evaluated using HI test (based on e.g. Kaufmann, L et al., JoVE 130: e55833 (2017)) with A/chicken/Irak/AV1342/2011 (Irak) antigen. In one embodiment, the mean H9-HA MDA level at the day of vaccination DO is 2.00 log 10, 2.50 log 10, 2.75 log 10, 3.00 log 10, 3.25 log 10, 3.50 log 10, 3.75 log 10 evaluated using HI test with A/chicken/Irak/AV1342/2011 (Irak) antigen.

The present invention also provides methods of immunizing, methods for eliciting an immune response and methods for eliciting a protective immune response in an animal using any of the recombinant vectors, immunogenic compositions, and/or vaccines described herein. In embodiments, the animal is an avian. In embodiments, the animal is a chicken.

In embodiments, the methods comprise administering to an animal a recombinant vector according to the present invention.

In embodiments, the methods comprise administering to an animal an immunogenic composition according to the present invention. In embodiments, the methods are effective to elicit, induce, and/or stimulate an immune response against MDV and/or AIV and/or IBDV or NDV in an animal.

In embodiments, the methods comprise administering to an animal a vaccine comprising an effective amount of a recombinant vector according to the present invention. In embodiments, the animal is vaccinated/immunized against MDV and/or AIV and/or IBDV or NDV. In embodiments, the methods are effective to elicit, induce, and/or stimulate a protective immune response against MDV and/or AIV and/or IBDV or NDV in an animal, and thereby reduce and/or prevent clinical signs associated with subsequent MDV and/or AIV and/or IBDV or NDV exposure, infection, challenge and/or disease in the animal, relative to a non-vaccinated control animal of the same species. In embodiments, the protective immune response is effective to provide the animal with protection against subsequent MDV and/or AIV and/or IBDV or NDV infection or challenge, and clinical disease and signs associated therewith.

In embodiments, the recombinant vectors, immunogenic compositions, and/or vaccines may be administered in ovo 1 to 4 days before hatching. In embodiments, the recombinant vectors, immunogenic compositions, and/or vaccines may be administered to a 1 day old, 2 day old, 3 day old, 4 day old, 5 day old, 6 day old, 7 day old, 8 day old, 9 day old, 10 day old, 11 day old, 12 day old, 13 day old, 14 day old, 15 day old, 16 day old, 17 day old, 18 day old, 19 day old, 20 day old, or 21 day old chicken. A variety of administration routes may be used such as aerosol (spray), ocular, nasal, oculonasal, oral, subcutaneous injection, and intramuscular injection. In an embodiment, the administration routes are used in 1 day old chicks.

In an embodiment, the animals that the recombinant HVT vectors of the invention are administered to are chickens. In an embodiment, the animals are SPF chickens. In an embodiment, the animals are 1 day old SPF chickens.

### Methods of Manufacture

In a twelfth aspect, the present invention provides a method of manufacturing the recombinant HVT vector of the invention, wherein the method comprises:
a) providing a nucleic acid encoding a HVT vector;
b) providing one or more expression cassettes comprising sequences encoding a first avian antigen and a second avian antigen, wherein the first or second avian antigen is an AIV H9-HA antigen; and
c) recombinantly combining the nucleic acid encoding a HVT vector and the one or more expression cassettes, such that the sequences encoding the first avian antigen and the second avian antigen are comprised in the same non-essential site in the HVT vector genome.

For the avoidance of doubt, in an embodiment, the method steps a), b) and c) are typically in this order (step a) followed by step b) followed by step c)).

In an embodiment, step c) involves recombinantly combining a HVT vector genome with a first expression cassette comprising a sequence encoding a first avian antigen and a second expression cassette comprising a sequence encoding a second avian antigen.

In an embodiment, step c) involves recombinantly combining a recombinant HVT vector genome with a first expression cassette comprising a sequence encoding a first avian antigen and a second expression cassette comprising a sequence encoding a second avian antigen.

In an embodiment, step c) involves recombinantly combining a HVT vector genome with an expression cassette comprising a sequence encoding a first avian antigen, further comprising a sequence encoding a second avian antigen.

In an embodiment, step c) involves recombinantly combining a recombinant HVT vector comprising a sequence encoding a first avian antigen with an expression cassette comprising a sequence encoding a second avian antigen.

In an embodiment, step c) involves recombinantly combining a recombinant HVT vector genome comprising a sequence encoding a second avian antigen with an expression cassette comprising a sequence encoding a first avian antigen.

In a preferred embodiment of this aspect and these embodiments, the first avian antigen is different to the second avian antigen. Preferably, the second avian antigen is the AIV H9-HA antigen.

In embodiments, the methods comprise inserting a polynucleotide encoding an avian influenza H9 antigen into a HVT backbone vector. In embodiments, the H9 antigen is from strain A/avian/Saudi Arabia/910135/2006 (H9N2). In embodiments, the polynucleotide encoding the H9 antigen has been codon-optimised.

In embodiments, the methods further comprise inserting a promoter into the backbone vector. In embodiments, the polynucleotide encoding the first avian antigen is operably linked to a promoter, and expression of the first avian antigen is regulated by the promoter. In embodiments, the promoter is a mCMV IE promoter.

In embodiments, the methods further comprise inserting an IRES or P2A sequence into the backbone vector. In embodiments, the IRES or P2A links the polynucleotide encoding the first avian antigen to the polynucleotide encoding the second avian antigen.

In embodiments, the methods further comprise inserting a polyadenylation (polyA) signal into the backbone vector. In embodiments, the polyA signal is a simian virus 40 (SV40) polyA tail. In embodiments, the polyA signal is inserted downstream of the polynucleotide encoding the second avian antigen.

### General Definitions

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

It is noted that this disclosure may contain the terms “comprising,” “consisting essentially of,” and “consisting of,” and variants thereof, and these terms have the meaning attributed to them in U.S. patent law. It is understood that wherever embodiments are described herein with the language “comprising,” otherwise analogous embodiments described in terms of “consisting essentially of” and/or “consisting of” are also provided.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50 means in the range of 45-55. Numerical ranges recited herein by endpoints include all numbers and fractions subsumed within that range (e.g. 1 to 5 includes 1, 1.5, 2, 2.75, 3, 3.90, 4, and 5).

As used herein, an “adjuvant” is a substance that is able to favor or amplify the cascade of immunological events, ultimately leading to a better immunological response (e.g., the integrated bodily response to an antigen). An adjuvant is in general not required for the immunological response to occur, but favors or amplifies this response.

As used herein, the terms “antigen” or “immunogen” mean a substance that induces a specific immune response in a host animal (e.g., an immune response of the humoral and/or cellular type directed against the antigen). The antigen may be a whole organism, killed, attenuated or live; a subunit or portion of an organism; a recombinant vector containing an insert with immunogenic properties; a piece or fragment of DNA capable of inducing an immune response upon presentation to a host animal; a polypeptide, an epitope, a hapten, and the like.

As used herein, the term “avian” includes, for example, chicken, chick, broiler, capon, duck, goose, turkey, grouse, quail, swan, squab, pigeon, pheasant, parrot, finches, hawk, crow, ostrich, emu and cassowary. The term “avian” also includes an individual avian in all stages of development, including embryonic and fetal stages. In a preferred embodiment, an avian is a chicken.

As used herein, the term “carrier” refers to a solvent or diluent in which a recombinant vector is formulated and/or administered. Pharmaceutically and veterinarily acceptable carriers can be sterile liquids such as water and/or oils. Examples of suitable oil-based carriers can include petroleum oils, animal oils, vegetable oils, and oils of synthetic origin (e.g., peanut oil, soybean oil, mineral oil, sesame oil, etc.). Examples of suitable aqueous carriers can include water and aqueous solutions (e.g. aqueous saline solution, aqueous dextrose solution, glycerol solution, etc.).

Herein it will be understood that “fragments thereof” relate to polypeptide or polynucleotide sequences having an equivalent function to the polypeptide or polynucleotide sequence in respect of which they are defined by SEQ ID NO. In an embodiment, equivalent function means that a polynucleotide fragment encodes an AIV H9-HA antigen. In an embodiment in respect of AIV H9-HA antigen sequences, equivalent function means that a polypeptide fragment comprises an AIV H9-HA antigen. In an embodiment, an AIV H9-HA antigen is defined as a sequence that is capable of inducing an immune response, preferably a protective immune response, against AIV, preferably H9N2 AIV. In an embodiment in respect of mCMV IE promoter sequences, equivalent function means that a polypeptide fragment comprises a functional promoter fragment. In an embodiment, a functional promoter fragment is defined as a sequence that is capable of inducing a similar or equivalent level of expression to the mCMV IE promoter. In an embodiment in respect of IBDV VP2 antigen sequences, equivalent function means that a polypeptide fragment comprises an IBDV VP2 antigen. In an embodiment, an IBDV VP2 antigen is defined as a sequence that is capable of inducing an immune response, preferably a protective immune response, against IBDV. In an embodiment in respect of NDV F antigen sequences, equivalent function means that a polypeptide fragment comprises a NDV F antigen. In an embodiment, a NDV F antigen is defined as a sequence that is capable of inducing an immune response, preferably a protective immune response, against NDV.

As used herein, the term “gene” is used broadly to refer to any segment of polynucleotide associated with a biological function.

As used herein, the term “genome” refers to the heritable genetic information of a host organism. The genome contemplated in the present invention can refer to the DNA or RNA of a virus or pathogenic organism. The RNA may be a positive strand or a negative strand RNA. In an embodiment in respect of HVT, the genome is dsDNA.

As used herein, the term “heterologous” means derived from a genetically distinct entity from the rest of the entity to which it is being compared. For example, a polynucleotide may be placed by genetic engineering techniques into a plasmid or vector derived from a different source, and is a heterologous polynucleotide. A promoter removed from its native coding sequence and operatively linked to a coding sequence other than the native sequence is a heterologous promoter.

As used herein, the terms “identity” and “sequence identity” refer to a relationship between two or more sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides/amino acids are identical. The total number of such position identities is then divided by the total number of nucleotides/amino acids in the shorter of the two sequences to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48:1073 (1988). Preferred methods to determine the sequence identity are designed to give the largest match between the sequences tested. Methods to determine sequence identity are also codified in publicly available computer programs which determine sequence identity between given sequences.

As used herein, the term “immunogenic composition” refers to a composition that comprises at least one antigen which elicits an immunological response in a host to which the immunogenic composition is administered.

As used herein, a “protective immune response” comprises an “immunological response” to a composition or vaccine which is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display a protective or therapeutic immunological response such that resistance to new infection will be enhanced and/or the clinical severity of subsequent disease reduced. Such protection can be demonstrated by a reduction in clinical disease signs relative to those normally displayed by an infected host, a lack of clinical disease signs relative to those normally displayed by an infected host, a quicker recovery time relative to that normally displayed by an infected host, and/or a lowered pathogen count relative to that normally found in an infected host.

As used herein, the terms “nucleic acid” and “polynucleotide” refer to RNA, DNA, and derivatives thereof.

As used herein, the term “codon optimized” refers to a polynucleotide that is genetically engineered to increase protein expression from the polynucleotide in a given species.

As used herein, the terms “pharmaceutically acceptable” and “veterinarily acceptable” are used adjectivally to mean that the modified noun is appropriate for use in a pharmaceutical or veterinary product. When it is used, for example, to describe an excipient in a pharmaceutical or veterinary vaccine, it characterizes the excipient as being compatible with the other ingredients of the composition and not disadvantageously deleterious to the intended recipient.

As used herein, the terms “polypeptide” and “protein” are used interchangeably to refer to a polymer of consecutive amino acid residues.

As used herein, the term “protection” does not require complete protection from any indication of infection. For example, protection can mean that, after challenge, clinical signs of the underlying infection are at least reduced, and/or that one or more of the underlying cellular, physiological, or biochemical causes or mechanisms causing the clinical signs are reduced and/or eliminated. It is understood that reduced, as used in this context, means relative to the state of the infection, including the molecular state of the infection, not just the physiological state of the infection.

As used herein, the term “recombinant” in the context of a polynucleotide or protein means a polynucleotide or protein with a semisynthetic or synthetic origin which either does not occur in nature or is linked to another a polynucleotide or protein in an arrangement not found in nature. A recombinant polynucleotide or protein can be made by modifying, altering, or engineering a polynucleotide or protein from its native form or structure to a non-native form or structure. The modification, alteration or engineering of a polynucleotide or protein may include, for example, the deletion of one or more nucleotides or amino acids, the substitution of one or more nucleotides or amino acids, and/or the insertion of one or more nucleotides or amino acids.

As used herein, a “vaccine” is an immunogenic composition that is suitable for administration to an animal which, upon administration to the animal, induces an immune response strong enough to aid in the protection from a clinical disease arising from an infection with a wild-type pathogenic micro-organism (e.g., strong enough for aiding in the curing of, ameliorating of, protection against, and/or prevention of a clinical disease and/or clinical signs associated therewith).

The invention will now be further described by way of the following non-limiting examples.

### EXAMPLES

### Example 1: Construction of vHVT513 Expressing IBDV VP2 and AIV H9

This Example details the construction and stability of a recombinant turkey herpesvirus (HVT) virus containing a murine cytomegalovirus (mCMV) IE promoter, a sequence encoding an infectious bursal disease virus (IBDV) VP2 capsid protein, an internal ribosome entry site (IRES), a sequence encoding a hemagglutinin protein (H9), and a simian virus 40 (SV40) poly A tail in the Intergenic I site in the BamHI I fragment (vHT513).

A. Genes for Insertion into the HVT Vector

Hemagglutinin Protein H9: A synthetic Saudi Arabia codon optimized Hemagglutinin Protein H9 (Genbank Accession #ACY80655.1). The protein has the amino acid sequence according to SEQ ID NO: 1 and is encoded by the nucleotide sequence according to SEQ ID NO: 9.

IBDV capsid protein VP2: A synthetic Infectious Bursal Disease Virus structural capsid protein VP2. The protein has the amino acid sequence according to SEQ ID NO: 2 and is encoded by the nucleotide sequence according to SEQ ID NO: 10.

B. Description of Donor Plasmid pSaH9optIRESVP2

Plasmid backbone: A synthetically ordered plasmid (pFIRESVP2) containing the right Intergenic I arm of vHVT013 (Vaxxitek HVT+IBD), SV40 poly A tail, Newcastle fusion protein, IRES, and Infectious Bursal Disease Virus capsid protein, where the Newcastle fusion protein was replaced with the Hemagglutinin H9 protein.

Promoter: mCMV IE promoter from the parent vHVT013 virus.

Antibiotic Resistance genes: Ampicillin.

C. Construction of Donor Plasmid pSaH9optIRESVP2

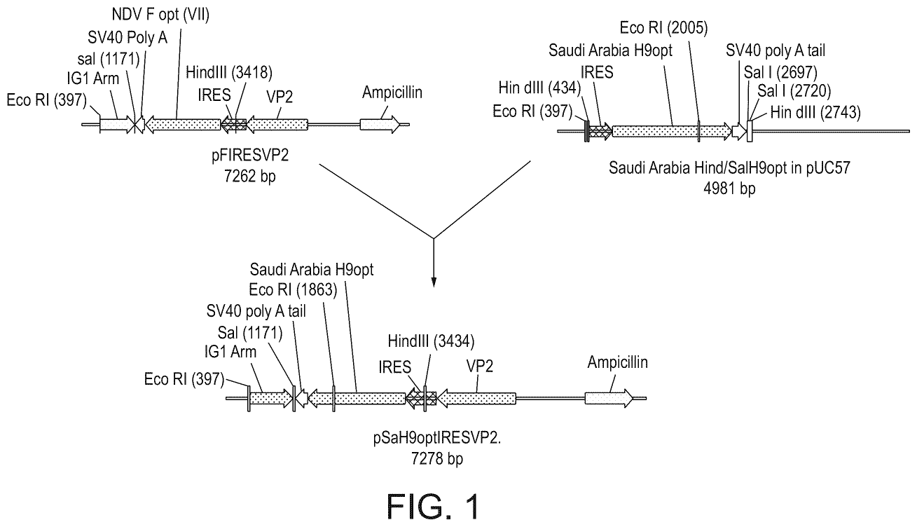

Diagram of plasmid construction and plasmid: FIG. 1 shows a diagram of the plasmid construction and pSaH9optIRESVP2.

Detailed description of plasmid construction: pFIRESVP2 was digested with HindIII and SalI. The 5 kb vector was gel extracted. Saudi Arabia Hind/SalH9opt in pUC57 was also digested with HindIII and SalI. The 2.2 kb insert was gel extracted. The 5 kb vector and 2.2 kb inserted were ligated together and transformed into Top Ten Competent cells. The resulting plasmids were screened by an EcoRI digestion. pSaH9optIRESVP2 was verified by sequence analysis and has the sequence according to SEQ ID NO: 3.

D. Description of Recombinant vHVT513

Parental virus: vHVT013 genomic DNA was extracted and used as the parental virus for in vitro homologous recombination.

Insertion locus: The same a vHVT013 designated intergenic I locus located in the BamHI-I fragment (Bublot et al., 1999).

Donor plasmid: pSaH9optIRESVP2.

Cells for in vitro recombination: Secondary chicken embryo fibroblast cells (2° CEF).

Method for recombinant selection: Immunofluorescence using hemagglutinin H9 polyclonal chicken sera and PCR.

E. Materials and Methods for Constructing Recombinant vHVT513

Detailed description of recombinant generation: vHVT513 recombinant was obtained by in vitro recombination between pSaH9optIRESVP2 plasmid and vHVT013 as previously described (Darteil et al. 1995 Virology 211, 481-490). Briefly, CEFs were co-transfected with pSaH9optIRESVP2 plasmid and vHVT013 DNAs. H9-expressing plaques were identified by IFA and further plaque purified by limiting dilution in 96 well plates. After 96 well duplication, virus from a well that had 100% of plaques expressing H9 and that was negative for the parental vHVT013 by PCR was amplified in several passages in CEFs. This virus batch was designated vHT513 pre-MSV. The preMSV batch was further passaged 13 times in CEFs and the resulting batch was designated vHVT513 pre-MSV+13.

PCR Analysis of recombinant: DNA was extracted from vHVT513 pre-MSV and vHVT513 pre-MSV+13 by phenol/chloroform extraction, ethanol precipitated, and resuspended in 20 mM HEPES. PCR primers were designed to specifically identify the presence of the codon optimized Saudi Arabia H9, the IBDV VP2, the mCMV IE promoter, the SV40 poly A tail, as well as the purity of the recombinant virus from the vHVT013 parental virus (Table 1). PCR was performed using 200 ng of DNA template along with the specified primer pairs indicated in Table 1. PCR cycling conditions are as follows: 94° C.-2 mins; 30 cycles of 94° C.-30 secs, 60° C.-45 secs, 68° C.-3 or 5 mins; 68° C.-5 or 7 mins (the annealing and extension times were adjusted according to the expected product size).

Expression Analysis of recombinant: Immunofluorescence testing was performed using vHVT513 pre-MSV and vHVT513 pre-MSV+13. The pre-MSV and pre-MSV+13 materials were diluted 1:100 in media. 50 μl of the diluted virus was added to 10 ml of DMEM+2% FBS with 1×10⁷ CEFs and then aliquoted onto a 96-well pate (100 μl/well). The plates were incubated for 3 days at 37° C.+5% CO₂ until viral plaques were visible. The plates were fixed with 95% ice-cold acetone for three minutes and washed three times with water. The plates were tested for the dual expression of HVT and IBDV-VP2 using an HVT monoclonal antibody L-78 (Lee et al. 1983 J Immunol 130, 1003-6) at 1:3000 and chicken anti-serum against Infectious Bursal Disease Virus (Charles Rivers Laboratory) at 1:500. The plates were also tested for the dual expression of IBDV-VP2 and Avian Influenza H9 using an IBDV-VP2 monoclonal antibody (1C6 Darteil et al. 1995 Virology 211,481-90) at 1:2000 and chicken anti-serum against Avian Influenza H9 (Charles Rivers Laboratory) at 1:300. All plates were incubated at 37° C. for 45 minutes. After the incubation the plates were washed three times with PBS and FITC anti-chicken (cat #F8888, Sigma) was added at 1:500 along with Alexa Fluor 568 donkey anti-mouse (IgG) (cat #A10037, Life Technologies) at 1:300. Again the plates were incubated at 37° C. for 45 minutes. After the incubation, the plates were washed three times with PBS. A small amount of PBS was added to prevent the monolayer from drying. The cells were then visualized with a fluorescent microscope using both the tetramethylrhodamine isothiocyanate (TRITC) and fluorescein isothiocyanate (FITC) filters.

Genomic Analysis of recombinant: Analysis of the pre-MSV and pre-MSV+13 genomic DNA was performed by PCR amplification. Primers 110P-1 and 309P18 were used to amplify the entire cassette, as well as beyond the flanking arms used in the generation of vHVT013. PCR cycling conditions were as follows: 94° C.-2 mins; 30 cycles of 94° C.-30 secs, 60° C.-45 secs, 68° C.-8 mins; 68° C.-10 mins. A 7.7 kb PCR product was gel purified using Qiagen's Gel Extraction Kit and the entire fragment was sequenced using the sequencing primers listed in Table 3.

F. Results from Analysis of Recombinant vHVT513

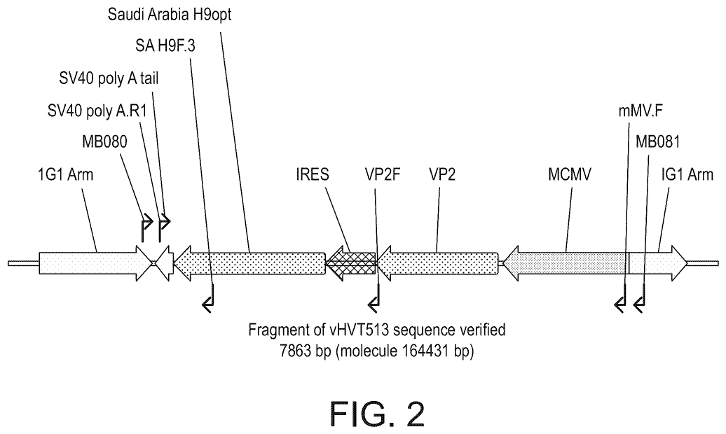

PCR Analysis of recombinant: PCR analysis of vHVT513 pre-MSV and pre-MSV+13 was performed using the PCR primers listed in Table 1 and shown on the vHVT513 map in FIG. 2. It was confirmed that the sizes of the PCR products after gel electrophoresis corresponded well with the expected sizes and the banding patterns. PCR reactions with all primer pairs resulted in the expected PCR products and banding patterns. There was no evidence of the parental vHVT013 virus in vHVT513

TABLE 1
vHVT513 Primers' sequences and expected sizes (in base pairs) of the
PCR-amplified fragments using different pairs of primers
either on vHVT013 or vHVT513 DNA template.

| Primer | Primer Sequence 5′−>3′ | Primer Set | vHVT013 | vHVT513 |
|---|---|---|---|---|
| MB080 | CGA ACA AAC TTC ATC GCT | MB080 + MB081 | 3350 | 5598 |
| | ATG C (SEQ ID NO: 15) | | | |
| MB081 | TAA CTC AAA TGC GAA GCG | MB080 + SAH9F.3 | — | 808 |
| | TTG C (SEQ ID NO: 16) | | | |
| SAH9F.3 | CTC TGA AGT TGA AAC CCG | MB080 + VP2.F | 405 | 2653 |
| | TC (SEQ ID NO: 17) | | | |
| VP2.F | AGA TTG CAG GAG CAT TTG | SV40polyA.R1 + mCMVF | 2959 | 5195 |
| | GCT (SEQ ID NO: 18) | | | |
| SV40polyA.R1 | ATG CAA TTG TTG TTA ACT | | | |
| | (SEQ ID NO: 19) | | | |
| mCMVF | AAC TCC GCC CGT TTT ATG | | | |
| | (SEQ ID NO: 20) | | | |

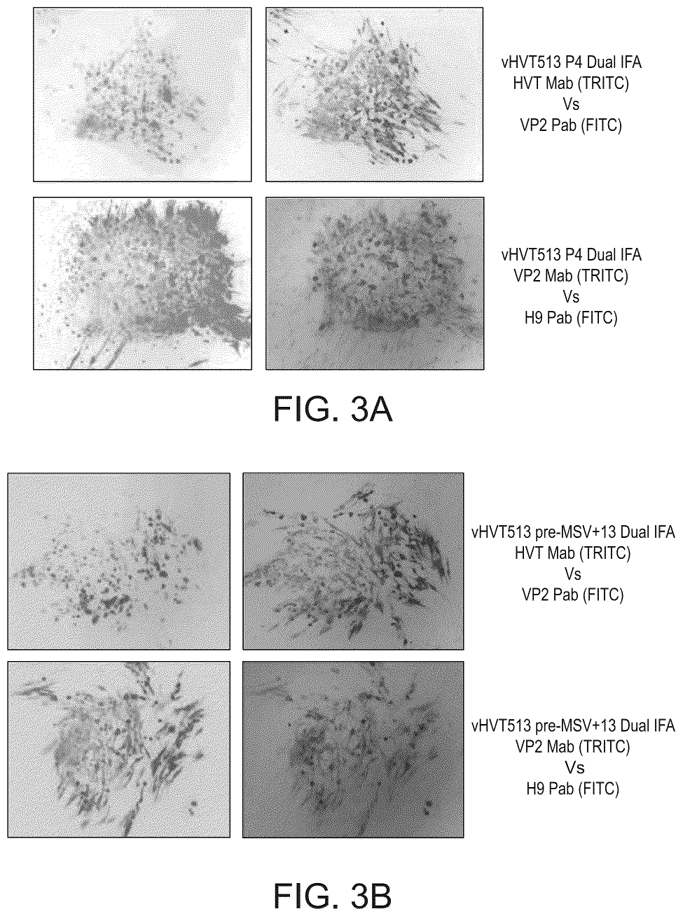

Expression Analysis of recombinant: Recombinant vHVT513 viral plaques were visualized using both the TRITC and FITC filters for the dual staining. For both the pre-MSV and pre-MSV+13 the TRITC showed the HVT expression or IBDV-VP2 monoclonal antibody and the FITC showed the chicken anti-sera expression of VP2 or H9. Because of the small wells of the 96-well plates, each well was recorded with the plaques first counted with the TRITC filter and then recounted with the FITC filter. 389 plaques were counted comparing the HVT TRITC and the VP2 FITC expressing plaques and 411 plaques were counted comparing the VP2 TRITC and the AIV-H9 FITC expressing plaques for the pre-MSV. All the plaques were positive for both the TRITC and FITC on both plates (refer to FIGS. 3A-3B). The same was done for the pre-MSV+13. Comparing HVT with VP2, 351 plaques were counted and comparing VP2 and AIV-H9, 314 plaques were counted. All the plaques were positive for both the TRITC and FITC on both plates (refer FIGS. 3A-3B). A summary of these data is shown in Table 2.

TABLE 2
Number of plaques checked for dual expression of HVT and
IBDV VP2, and IBDV VP2 and AIV H9.

| Double staining | vHVT513 pre-MSV | vHVT513 pre-MSV + 13 |
|---|---|---|
| HVT/IBDV VP2 | 389/389 | 351/351 |
| IBDV VP2/AIV H9 | 411/411 | 314/314 |

Genomic Analysis of recombinant: Analysis of vHVT513 pre-MSV and vHVT513 pre-MSV+13 genomic DNA was performed by PCR amplification. Sequencing results obtained from vHVT513 pre-MSV were aligned. The alignment created exactly matched to the expected vHVT513 sequence. Sequencing results obtained from vHVT513 pre-MSV+13 were aligned. The alignment matched the exact sequence as vHVT513 pre-MSV.

Referring to primers 110P-1 and 309P18 in Table 3, these primers were used to amplify the entire cassettes, as well as, beyond the flanking recombination arms used in the donor plasmid and vHVT013 recombination arms. The 7.7 kb PCR product obtained was gel purified and the entire fragment was sequenced using the sequencing primers listed in Table 3.

TABLE 3
vHVT513 Sequencing Primers and Location.

| Primer | Sequence 5′ −>3′ | Primer | Sequence 5′−>3′ |
|---|---|---|---|
| 110P-1 | TCT TTG TTC CTT GAA ATG CCG ACA | 309P6 | ACT GAG TGA GAG GGA CCG TC |
| | (SEQ ID NO: 21) | | (SEQ ID NO: 32) |
| 110P1 | TCA TGT CGA TGA AAT GTT ATG | SAH9R | ATC AGG CTA ATT GGT TCC AT |
| | (SEQ ID NO: 22) | | (SEQ ID NO: 33) |
| 110P2 | TGC GTC GAT AAG ACT TTG GT | SAH9F | ATG GAA CCA ATT AGC CTG AT |
| | (SEQ ID NO: 23) | | (SEQ ID NO: 34) |
| 119P1 | TTA GTT TGC ATT TCT TAG GG | SAH9F.1 | ATC TTC CCG GAC ACC ATC TG |
| | (SEQ ID NO: 24) | | (SEQ ID NO: 35) |
| 119P2 | TGA ATA TTT GCA ACC AAT GC | SAH9F.2 | ACG TTC TGT CTG GTG AAT CT |
| | (SEQ ID NO: 25) | | (SEQ ID NO: 36) |
| mCMVP1 | TCA GCA CGG TCA TGC ACT CT | SAH9F.3 | CTC TGA AGT TGA AAC CCG TC |
| | (SEQ ID NO: 26) | | (SEQ ID NO: 17) |
| 309P1 | ATG TAC TTT CCC ACC ATT GAC G | 309P15R | ACA CGC GCC GTT GAG TAT |
| | (SEQ ID NO: 27) | | (SEQ ID NO: 37) |
| 309P2 | AGG CGG CCG CTC TAG AAC | 309P15 | ATA CTC AAC GGC GCG TGT |
| | (SEQ ID NO: 28) | | (SEQ ID NO: 38) |
| 309P3 | ACC GGC CAG CTA CAA CTA | 309P16 | ATC CGA AGA AGA AGG CGT |
| | (SEQ ID NO: 29) | | (SEQ ID NO: 39) |
| 309P4 | ATG CGA CAG CAG TGA CAG | 309P18 | ACC GCC AGA TAT CCA AAG TA |
| | (SEQ ID NO: 30) | | (SEQ ID NO: 40) |
| 309P5 | AGA TAA CCC AGC CAA TCA | | |
| | (SEQ ID NO: 31) | | |

Sequencing contigs were assembled from the sequencing reactions from the primers indicated on Table 3. The vHVT513 has the sequence as set forth in SEQ ID NO: 4.

G. Conclusion from Analysis of Recombinant vHVT513

Based on PCR, Immunofluorescence, and sequence analysis of vHVT513 there is no evidence of parental vHVT013 remaining. The data demonstrates that vHVT513 is a recombinant HVT expressing the VP2 gene from IBDV and a codon optimized Saudi Arabia H9 gene from Avian Influenza under the control of a mCMV IE promoter and an IRES sequence. The data also demonstrates that vHVT513 is stable genetically and phenotypically after 13 in vitro passages beyond the pre-MSV.

### Example 2: Construction of vHVT514 Expressing NDV-F and AIV H9

This Example details the construction and stability of a recombinant turkey herpesvirus (HVT) virus containing a murine cytomegalovirus (mCMV) IE promoter, a sequence encoding a Newcastle Disease virus fusion protein (NDV-Fwt), an internal ribosome entry site (IRES), a sequence encoding a hemagglutinin protein (H9), and a simian virus 40 (SV40) poly A tail in the Intergenic I site in the BamHI I fragment (vHT514).

A. Genes for Insertion into HVT Vector

Newcastle Disease virus fusion protein: A synthetic Newcastle Disease Virus Fusion Protein (NDV-F) corresponding to a consensus wild-type sequence of genotype VIId isolates was used as one of the inserts. It is 99.6% (551/553 amino acids) identical and 99.8% (552/553) similar to a goose velogenic strain (goose/Jiangsu/JSG0210/2002) isolated in China (GenBank accession #AEM55586.1). The F amino acid sequence was modified at the F2/F1 cleavage site from a velogenic site (GRRQKR/F) to a lentogenic site (GGKQGR/L). The protein has the amino acid sequence according to SEQ ID NO: 5 and is encoded by the nucleotide sequence according to SEQ ID NO: 11.

Hemagglutinin Protein H9: Another insert used is a synthetic Saudi Arabia codon optimized Hemagglutinin Protein H9 (GenBank Accession #ACY80655.1). The protein has the amino acid sequence according to SEQ ID NO: 1 and is encoded by the nucleotide sequence according to SEQ ID NO: 9.

B. Description of Donor Plasmid pFwtIRESSaH9opt

Plasmid backbone: A previously created plasmid (pFwtIRESgD) containing the right Intergenic I arm of vHVT013, Newcastle fusion protein, IRES, Infectious Laryngotracheitis Disease Virus glycoprotein D, SV40 poly A tail, and the left Intergenic I arm of vHVT013. The Laryngotracheitis Disease Virus glycoprotein D was replaced with the Hemagglutinin H9 protein. The plasmid is insertable into the Intergenic I (IG1) site.

Promoters: mCMV IE promoter.

Antibiotic Resistance genes: Ampicillin.

C. Construction of Donor Plasmid pFwtIRESSaH9opt

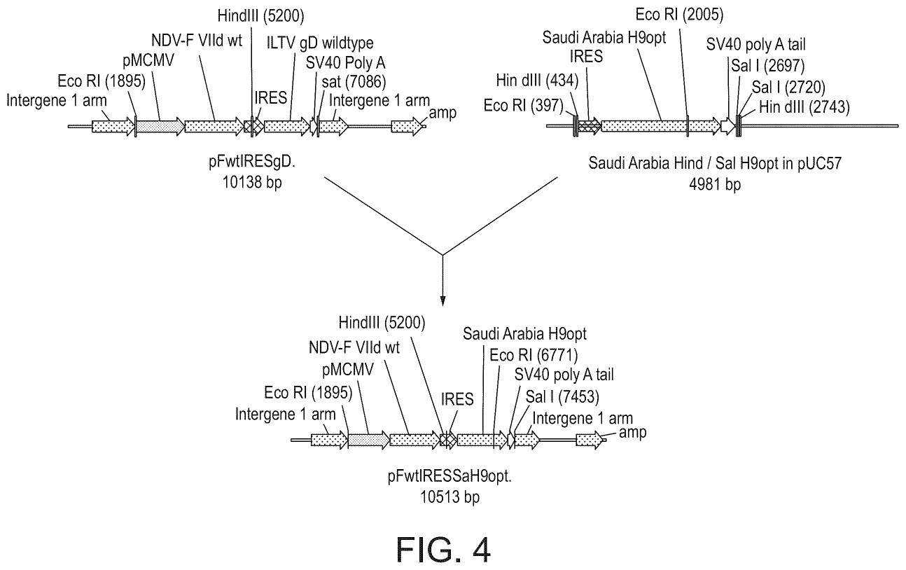

Diagram of plasmid construction and plasmid: FIG. 4 shows a diagram of the plasmid construction and pFwtIRESSaH9opt.

Detailed description of plasmid construction: pFwtIRESgD was digested with HindIII and SalI. The 8.2 kb vector was gel extracted. Saudi Arabia Hind/SalH9opt in pUC57 was also digested with HindIII and SalI. The 2.2 kb insert was gel extracted. The 8.2 kb vector and 2.2 kb inserted were ligated together and transformed into Top Ten Competent cells. The resulting plasmids were screened by an EcoRI/HindIII digestion. pFwtIRESSaH9opt was verified by sequence analysis and has the sequence according to SEQ ID NO: 6.

D. Description of Recombinant vHVT514

Parental virus: vHVT013 genomic DNA was extracted and used as the parental virus for in vitro homologous recombination.

Insertion locus: Intergenic I locus located in the BamHI-I fragment (Bublot et al., 1999).

Donor plasmid: pFwtIRESSaH9opt (Newcastle Disease fusion protein-IRES-Saudi Arabia Avian Influenza H9 hemagglutinin protein-SV40 poly A tail).

Cells for in vitro recombination: Secondary chicken embryo fibroblast cells (2° CEF).

Method for recombinant selection: Immunofluorescence using hemagglutinin H9 polyclonal chicken sera and PCR.

E. Materials and Methods for Constructing Recombinant vHVT514

Detailed description of recombinant generation: vHVT514 recombinant was obtained by in vitro recombination between pFwtIRESSaH9opt plasmid and vHVT013 as previously described (Darteil et al. 1995 Virology 211, 481-490). Briefly, CEFs were co-transfected with pFwtIRESSaH9opt plasmid and vHVT013 DNAs. H9-expressing plaques were identified by IFA and further plaque purified by limiting dilution in 96 well plates. After 96 well duplication, virus from a well that had 100% of plaques expressing H9 and that was negative for the parental vHVT013 by PCR was amplified in several passages in CEFs. This virus batch was designated vHT514 pre-MSV. The preMSV batch was further passaged 13 times in CEFs and the resulting batch was designated vHVT514 pre-MSV+13.

PCR Analysis of recombinant: DNA was extracted for vHVT514 pre-MSV and vHVT514 pre-MSV+13 by phenol/chloroform extraction, ethanol precipitated, and resuspended in 20 mM HEPES. PCR primers were designed to specifically identify the presence of the codon optimized Saudi Arabia H9, the NDV-F, the mCMV IE promoter, the SV40 poly A tail, as well as the purity of the recombinant virus from the vHVT013 (Vaxxitek parental virus, Table 4). PCR was performed using 200 ng of DNA template along with the specified primer pairs indicated in Table 4. PCR cycling conditions are as follows: 94° C.-2 mins; 30 cycles of 94° C.-30 secs, 60° C.-45 secs, 68° C.-3 or 5 mins; 68° C.-5 or 7 mins (the annealing and extension times were adjusted according to the expected product size).

Expression Analysis of recombinant: Immunofluorescence testing was performed using vHVT514 pre-MSV and vHVT514 pre-MSV+13. The pre-MSV and pre-MSV+13 materials were diluted 1:100 in media. 50 μl of the diluted virus was added to 10 ml of DMEM+2% FBS with 1×10⁷ CEFs and then aliquoted onto a 96-well pate (100 μl/well). The plates were incubated for 3 days at 37° C.+5% CO₂ until viral plaques were visible. The plates were fixed with 95% ice-cold acetone for three minutes and washed three times with water. The plates were tested for the dual expression of HVT and NDV-F using an HVT monoclonal antibody L-78 (072103, Merial) at 1:3000 and chicken anti-serum against Newcastle Disease Virus (Charles Rivers Laboratory) at 1:500. The plates were also tested for the dual expression of NDV-F and Avian Influenza H9 using NDV ascetic fluid (1C3, Meulemans et al 1987 Arch Virol 92, 55-62) at 1:1000 and chicken anti-serum against Avian Influenza H9 (Charles Rivers Laboratory) at 1:300. All plates were incubated at 37° C. for 45 minutes. After the incubation the plates were washed three times with PBS and FITC anti-chicken (cat #F8888, Sigma) was added at 1:500 along with Alexa Fluor 568 donkey anti-mouse (IgG) (cat #A10037, Life Technologies) at 1:300. Again the plates were incubated at 37° C. for 45 minutes. After the incubation, the plates were washed three times with PBS. A small amount of PBS was added to prevent the monolayer from drying. The cells were then visualized with a fluorescent microscope using both the tetramethylrhodamine isothiocyanate (TRITC) and fluorescein isothiocyanate (FITC) filters.

Genomic Analysis of recombinant: Analysis of the pre-MSV and pre-MSV+13 genomic DNA was performed by PCR amplification. Primers 110P-1 and 309P18 were used to amplify the entire cassette, as well as beyond the flanking arms used in the creation of vHVT013. PCR cycling conditions were as follows: 94° C.-2 mins; 30 cycles of 94° C.-30 secs, 60° C.-45 secs, 68° C.-8 mins; 68° C.-10 mins. An 8 kb PCR product was gel purified using Qiagen's Gel Extraction Kit and the entire fragment was sequenced using the sequencing primers listed in Table 6.

F. Results from Analysis of Recombinant vHVT514

PCR Analysis of recombinant: PCR analysis of vHVT514 pre-MSV and pre-MSV+13 was performed using the PCR primers listed in Table 4. It was confirmed that the sizes of the PCR products. PCR reactions with all primer pairs resulted in the expected PCR products and banding patterns. There was no evidence of the parental vHVTO13 virus in vHVT514.

TABLE 4
vHVT514 Primers’ sequences and expected sizes (in base pairs)
of the PCR-amplified fragments using different pairs of primers
either on vHVT013 or vHVT514 DNA template.

| Primer | Primer Sequence 5′−>3′ | Primer Set | vHVT013 | vHVT514 |
|---|---|---|---|---|
| MB080 | CGA ACA AAC TTC ATC GCT | MB080 + MB081 | 3350 | 5873 |
| | ATG C (SEQ ID NO: 15) | | | |
| MB081 | TAA CTC AAA TGC GAA GCG | MB080 + SAH9F.3 | — | 799 |
| | TTG C (SEQ ID NO: 16) | | | |
| SAH9F.3 | CTC TGA AGT TGA AAC CCG | MB080 + 312P6 | — | 2854 |
| | TC (SEQ ID NO: 17) | | | |
| 312P6 | CGT CAA CAA TTC AAT CAG | SV40polyA.R1 + mCMVF | 3021 | 5482 |
| | CA (SEQ ID NO: 41) | | | |
| SV40polyA.R1 | ATG CAA TTG TTG TTA ACT | | | |
| | (SEQ ID NO: 19) | | | |
| mCMVF | AAC TCC GCC CGT TTT ATG | | | |
| | (SEQ ID NO: 20) | | | |

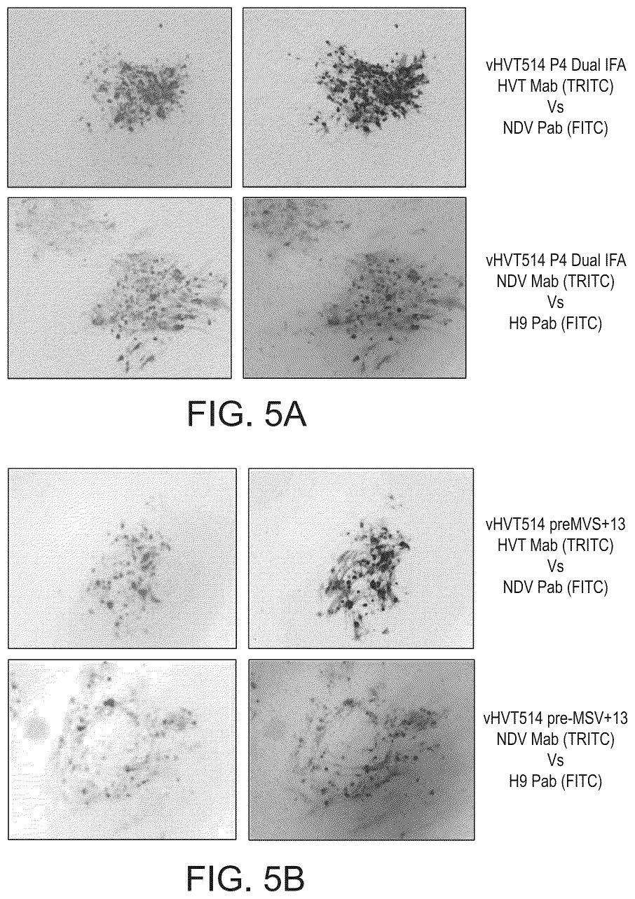

Expression Analysis of recombinant: Recombinant vHVT514 viral plaques were visualized using both the TRITC and FITC filters for the dual staining. For both the pre-MSV and pre-MSV+13 the TRITC showed the HVT expression or NDV-F monoclonal antibody and the FITC showed the chicken anti-sera expression of NDV or H9. Because of the small wells of the 96-well plates, each well was recorded with the plaques first counted with the TRITC filter and then recounted with the FITC filter. 405 plaques were counted comparing the HVT TRITC and the NDV FITC expressing plaques and 361 plaques were counted comparing the NDV TRITC and the AIV-H9 FITC expressing plaques for the pre-MSV. All the plaques were positive for both the TRITC and FITC on both plates (refer to FIGS. 5A-5B). The same was done for the pre-MSV+13. Comparing HVT with NDV, 405 plaques were counted and Comparing NDV and AIV-H9, 401 plaques were counted. All the plaques were positive for both the TRITC and FITC on both plates (refer to FIGS. 5A-5B). A summary of these data is shown in Table 5.

TABLE 5
Number of plaques checked for dual expression of HVT
and NDV-F, and NDV F and AIV-H9.

| Double staining | vHVT514 pre-MSV | vHVT514 pre-MSV + 13 |
|---|---|---|
| HVT/NDV-F | 405/405 | 405/405 |
| NDV-F/AIV-H9 | 361/361 | 401/401 |

Genomic Analysis of recombinant: Analysis of vHVT514 pre-MSV and vHVT514 pre-MSV+13 genomic DNA was performed by PCR amplification. Referring to primers 110P-1 and 309P18 in Table 6, these primers were used to amplify the entire cassettes, as well as beyond the flanking recombination arms used in the donor plasmid and vHVT013 recombination arms. The 8 kb PCR product obtained was gel purified and the entire fragment was sequenced using the sequencing primers listed in Table 6. Sequencing results obtained from vHVT514 pre-MSV were aligned. The alignment created exactly matched to the expected vHVT514 sequence. Sequencing results obtained from vHVT514 pre-MSV+13 were aligned. The alignment matched the exact sequence as vHVT514 pre-MSV.

TABLE 6
vHVT514 Sequencing Primers and Location.

| Primer | Sequence 5′−>3′ | Primer | Sequence 5′−>3′ |
|---|---|---|---|
| 110P-1 | TCT TTG TTC CTT GAA ATG CCG ACA | 312P6 | CGT CAA CAA TTC AAT CAG CA |
| | (SEQ ID NO: 21) | | (SEQ ID NO: 41) |
| 110P1 | TCA TGT CGA TGA AAT GTT ATG | SAH9R | ATC AGG CTA ATT GGT TCC AT |
| | (SEQ ID NO: 22) | | (SEQ ID NO: 33) |
| 110P2 | TGC GTC GAT AAG ACT TTG GT | SAH9F | ATG GAA CCA ATT AGC CTG AT |
| | (SEQ ID NO: 23) | | (SEQ ID NO: 34) |
| 119P1 | TTA GTT TGC ATT TCT TAG GG | SAH9F.1 | ATC TTC CCG GAC ACC ATC TG |
| | (SEQ ID NO: 24) | | (SEQ ID NO: 35) |
| 119P2 | TGA ATA TTT GCA ACC AAT GC | SAH9F.2 | ACG TTC TGT CTG GTG AAT CT |
| | (SEQ ID NO: 25) | | (SEQ ID NO: 36) |
| mCMVP1 | TCA GCA CGG TCA TGC ACT CT | SAH9F.3 | CTC TGA AGT TGA AAC CCG TC |
| | (SEQ ID NO: 26) | | (SEQ ID NO: 17) |
| 309P1 | ATG TAC TTT CCC ACC ATT GAC G | 309P15R | ACA CGC GCC GTT GAG TAT |
| | (SEQ ID NO: 27) | | (SEQ ID NO: 37) |
| 312P2 | TAT GCC CAG GGA TAA GGA GG | 309P15 | ATA CTC AAC GGC GCG TGT |
| | (SEQ ID NO: 42) | | (SEQ ID NO: 38) |
| 312P3R | ACT TCA TGC ACA GCT TCA | 309P16 | ATC CGA AGA AGA AGG CGT |
| | (SEQ ID NO: 43) | | (SEQ ID NO: 39) |
| 312P4 | CTG GTG GCA ATA TGG ATT | 309P18 | ACC GCC AGA TAT CCA AAG TA |
| | (SEQ ID NO: 44) | | (SEQ ID NO: 40) |
| 312P5R | ATG TGT TGC CGC TCA AAC AG | | |
| | (SEQ ID NO: 45) | | |

Sequencing contigs were assembled from the sequencing reactions from the primers indicated on Table 6. The vHVT514 has the sequence as set forth in SEQ ID NO: 7.

G. Conclusion from Analysis of Recombinant vHVT514

Based on PCR, Immunofluorescence, and sequence analysis of vHVT514 there is no evidence of parental vHVTO13 remaining. The data demonstrates that vHVT514 is a recombinant HVT expressing the fusion gene from NDV and a codon optimized Saudi Arabia H9 gene from Avian Influenza under the control of a mCMV IE promoter and an IRES sequence. The data also demonstrates that vHVT514 is stable genetically and phenotypically at 13 passages beyond the pre-MSV.

### Example 3: AIV Challenge Study

This Example details the efficacy assessment of several recombinant HVT candidate vaccines expressing the H9 of Avian Influenza H9N2, administered to one-day-old SPF chickens against an Avian Influenza H9N2 (Saudi Arabia 2010) virulent challenge carried out 21 days after vaccination.

### A. Objective, Study Design, Vaccines and Vaccination

The objective of this study was to assess the efficacy of several recombinant candidate vaccines expressing the H9 of Avian Influenza H9N2, when administered to SPF chicks at one day of age, against a H9N2 virulent challenge (strain Saudi Arabia 2010) carried out 21 days post-vaccination. To reach this objective, the following parameters were monitored after challenge: (i) respiratory and ocular symptoms (clinical monitoring); and (ii) oro-pharyngeal viral excretion (assessed by qRT-PCR).

On DO, SPF one-day-old chicks were identified, randomly allocated to different groups (12 birds per group) and treated with different HVT vaccine candidates all expressing the H9 gene from the A/avian/Saudi Arabia/910135/2006 strain, as indicated in table 7 below:

TABLE 7
Group constitution and treatment

| Group | Vaccine | Dose | Admin. route | volume |
|---|---|---|---|---|
| G1 | vHVT513 | 3.4 log10 PFU | S.C. | 0.2 mL |
| G2 | vHVT514 | 3.4 log10 PFU | S.C. | 0.2 mL |
| G3 | — | — | | — |

Generation of vHVT513 and vHVT514 is described in the previous examples. Vaccine doses are shown in Table 7. vHVT513 and vHVT514 were administered by the subcutaneous (SC) route after dilution in Marek's vaccine diluent.

### B. Serology

Blood sample was taken at D20 to evaluate the serological response induced by the vaccine candidates. Hemagglutination-inhibition (HI) test (based on e.g. Kaufmann, L et al., JoVE 130: e55833 (2017)) was used to evaluate the anti-H9 antibodies using an H9N2 antigen (A/chicken/Irak/AV1342/2011 H9N2).

Dispersions of individual HAI titers on D20 by groups are presented in FIG. 9.

On D20, all control animals were seronegative. At the same date, all animals from groups G1 and G2 had seroconverted.

### C. H9N2 Challenge and Clinical Protection

From DO to D21, no clinical sign was recorded in groups G1, G2 and G3 as part of daily observation. At D21, birds were challenged with the H9N2 A/chicken/Saudi Arabia/WNB9510/2010, also referred to herein as Saudi Arabia 2010. The challenge strain was diluted in physiological buffered saline (PBS) pH: 7.1 to obtain a challenge suspension with a titer of 9.0 log 10 EID50/ml (being 8.54 log 10 EID50/0.35 ml). All animals of groups G1, G2 and G3 were challenged with 0.35 ml of the challenge solution by nebulization. In detail, animals from the same group were gathered in a nebulization box and nebulized with 4.2 ml for about 20 minutes. Animals were kept in closed contact in the nebulization box for about 3 to 5 minutes and then, they were moved back to their isolators. Chickens of all groups were daily monitored from D21 (before challenge) to D31. For this monitoring, particular attention was paid to respiratory and ocular symptoms. All other observed clinical signs were recorded as well. Respiratory and ocular symptoms were scored as detailed in Table 8.

TABLE 8
Description and score of respiratory and ocular clinical signs.
If an animal was found dead or euthanized on ethical grounds
after challenge, it was attributed a score of 6 daily until the end
of the study, when clinical signs or necropsy result could be
associated to the H9N2 challenge.

| | Clinical signs | |
|---|---|---|
| Type | Description | Score |
| Respiratory | Slight: slight rale, sneezing | 1 |
| | Mild: severe rale, cough, tachypnea | 2 |
| | (increase of the respiratory frequency) | |
| | Severe: respiratory distress | 3 |
| Ocular | Slight: conjunctivitis with oedema of the | 1 |
| | eyelid and excessive weeping | |
| | Mild: conjunctivitis with oedema of the eyelid | 2 |
| | and excessive weeping + periorbital oedema | |
| | Severe: conjunctivitis with oedema of the | 3 |
| | eyelid and excessive seeping + | |
| | periorbital oedema + yellow thick exudate | |

For each animal, a daily clinical score was calculated by adding the scores associated to respiratory and ocular symptoms (see Table 8) from D22 to D31. For each animal, a global clinical score was calculated by adding all the daily clinical scores. Each group was compared to the control group on the criterion “Global clinical score” using a Mann-Whitney-Wilcoxon's test. On D31, all surviving animals were humanely euthanized.

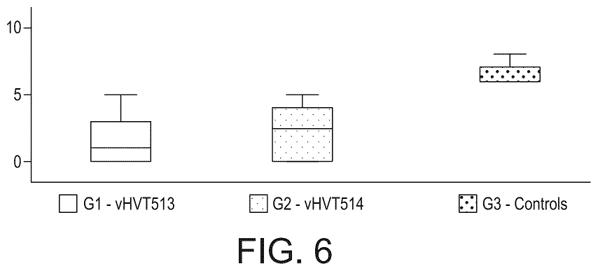

Dispersions of individual global clinical scores by groups are presented in FIG. 6. Following challenge on D21, mainly slight respiratory signs were recorded in the different groups. Rales were sometimes associated with crackling and whistling. The onset of the clinical signs was 2 days post challenge (dpc) in the control group and 4 dpc in groups G1 and G2. In detail: (i) six out of 12 animals presented clinical signs in group G1 (vHVT513); (ii) eight out of 12 animals presented clinical signs in group G2 (vHVT514), and (iii) 11 out of 12 animals presented clinical signs in group G3 (controls).

Global clinical scores in groups G1 and G2 were significantly lower than that of the control group (p=0.0005, and p=0.0010 respectively).

### D. H9N2 Shedding

On D25, D27 and D29 (4, 6 and 8 days post-challenge), an oro-pharyngeal swabbing was carried out on all chicks. The oro-pharyngeal H9N2 viral load was evaluated by qRT-PCR. When viral excretion was detected in a group, the area under curve (AUC) for the oro-pharyngeal H9N2 excretion from D25 to D29 was determined for each animal according to the trapezium method.

Virus-excreting groups were compared to the control group on the criterion “AUC” using a Student's T test (assuming the homogeneity of the variances (Fisher-Snedecor's test). For the analysis of viral excretions (AUC calculation), the animals found dead during the challenge period were attributed the last-observed viral excretion value for the next analyzed time points if lacking.

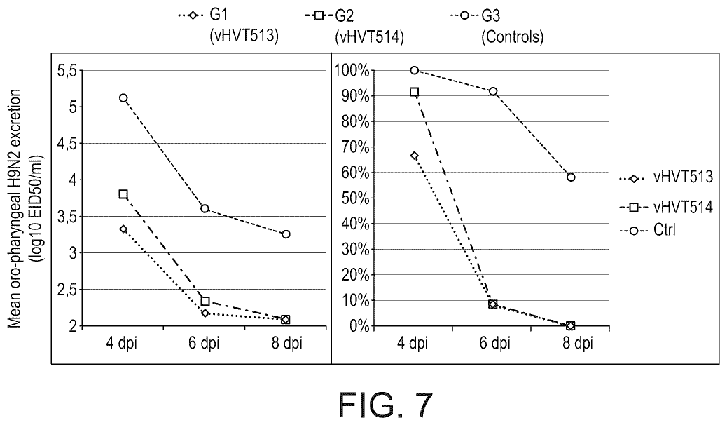

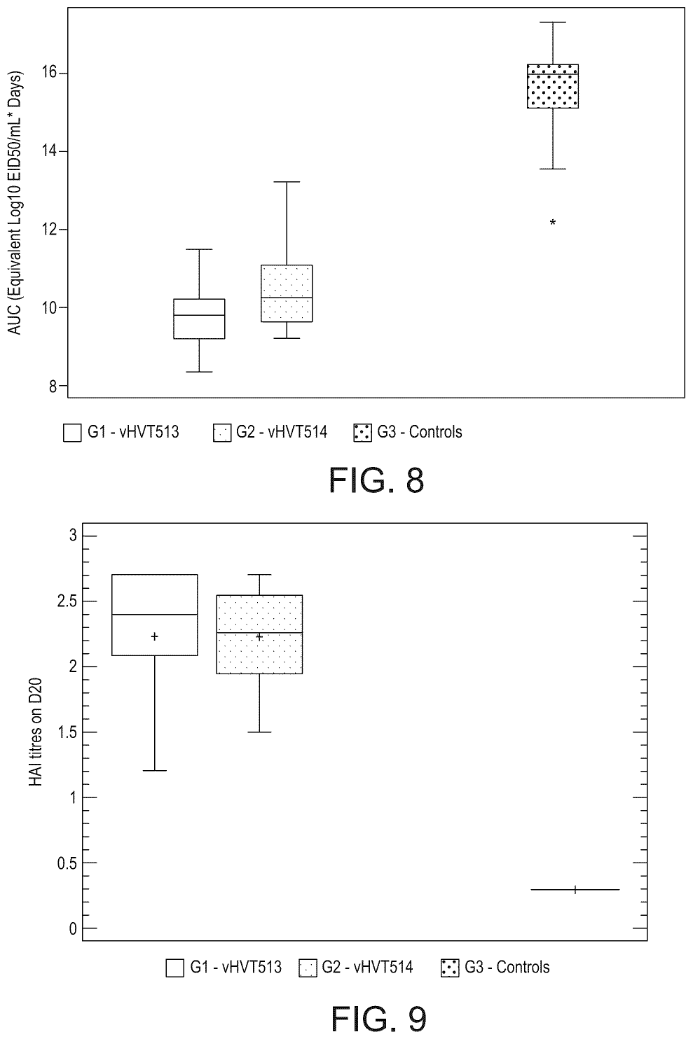

Evolutions of mean viral titers in oro-pharyngeal swabs and percentages of positive birds by groups are presented in FIG. 7. Dispersions of individual AUC of viral excretion by groups are presented in FIG. 8.

On D25 (4 days post-challenge) and D27 (6 days post-challenge), animals excreted virus in all groups with higher viral titers observed on D25 than on D27. On D29 (8 days post-challenge), no viral excretion was observed in groups G1 and G2, whereas animals carried on excreting virus in control group G3. All vaccinated groups had a reduction of percentage of positive swabs at the three time points which was higher at 6 and 8 days post-challenge. The lowest shedding level and percentage of positive was observed in G1 (vHVT513).

AUC of viral excretion in vaccinated groups G1 and G2 was significantly lower than that of the control group G3 (p<0.0001, and p<0.0001 respectively). The best reduction of shedding evaluated with AUC was for vHVT513, followed by vHVT514.

### E. Conclusion

Administration of the vHVT513 and vHVT514 candidates to one-day-old SPF chicks resulted in a 100% seroconversion to H9N2, and after H9N2 challenge at D21, in a significant decrease of both respiratory clinical signs and viral excretion.

### Example 4: AIV Challenge Study in Broilers with MDA

This Example details the protective effects of vHVT513 and vHVT514 against Avian Influenza H9N2 (AI H9N2) challenge in broilers with H9N2 maternally-derived antibodies (MDA) in comparison with an inactivated H9N2 vaccine.

### A. Objectives, Design of the Study, Vaccines and Vaccination

The objective of the study was to evaluate efficacy of two HVT vector vaccine candidates (vHVT513 and vHVT514) against an H9N2 challenge in broilers with H9N2 MDAs in comparison to an inactivated vaccine. The design of the experiment is shown in table 9.

TABLE 9
Design of the study.

| | | | Vaccination | Challenge |
|---|---|---|---|---|
| Group | Vaccine | Dose | day | day |
| G1 | vHVT513 | 3.4 log10 PFU | D0 | D21 |
| G2 | vHVT514 | 3.4 log10 PFU | D0 | D21 |
| G3 | Inact H9N2 | 0.2 mL | D7 | D21 |
| G4 | — | — | — | D21 |

One-day-old Cobb 500 broilers with H9N2 MDAs (10 per groups) were used in the study. Birds of groups G1 and G2 were vaccinated with vHVT513 and vHVT514 by the S.C. route on DO. Birds of group G3 were vaccinated with an inactivated H9N2 vaccine (GE208) that contains also an inactivated NDV antigen by the S.C. route.

### B. Serology

Blood sample was taken from 8 birds at DO to evaluate MDAs level and from all birds of G1 to G4 at D20 to evaluate MDAs decrease and antibodies induced by vaccination.

Anti-H9N2 antibodies were evaluated using HI test with A/chicken/Irak/AV1342/2011 (Irak) antigen and using a commercial ELISA Kit (ID Screen® Influenza H9 Antibody Competition ELISA from ID-Vet). Anti-NDV antibodies were evaluated using HI test with La Sota antigen and using a commercial ELISA Kit recognizing anti-F antibodies (ID-Vet). Anti-IBDV antibodies were evaluated using two ELISA PROFLOK IBD and PROFLOK PLUS IBD (Zoetis), the latter being able to detect anti-VP2 antibodies.

Mean H9N2 MDA levels at DO were 3.75 log 10±0.89 by HI test with Irak antigen.

Results of serology at D20 are shown in Table 10. Most birds of vaccinated groups induced H9N2 HI titers.

TABLE 10
Results of serology at D20. Mean titers in log10 +
standard deviation (number of positive/total).

| Groups | H9N2 HI |
|---|---|
| G1 (vHVT513) | 4.4 ± 2.6 |
| | (8/8) |
| G2 (vHVT514) | 4.4 ± 5.3 |
| | (9/10) |
| G3 (Inact. H9) | 5.3 ± 1.9 |
| | (9/10) |
| G4 (control) | 1.0 ± 0.0 |
| | (0/10) |

### C. H9N2 Challenge and Clinical Protection

At D21, all chicks were challenged with the A/chicken/Saudi Arabia/WNB9510/2010 H9N2 strain, also referred to herein as Saudi Arabia 2010. The diluent for the challenge strain was physiological buffered saline (PBS) pH: 7.1. This H9N2 challenge strain was thawed and diluted in PBS to obtain a challenge suspension with a titer 5.5 log 10 DIO50/0.35 ml. All birds were challenged with 0.35 ml of the challenge solution by nebulization. In detail, all animals from the same group were gathered in a nebulization box (plastic box, size [L50/139.5/h33 cm]) and nebulized for about 20 minutes with a 347-CN003 nebulizer. Animals were kept in closed contact in the nebulization box for about 3 to 5 minutes and then, they were moved back to their isolators.

Chickens of all groups were daily monitored from D21 (before challenge) until the end of the study (which was D31). For this monitoring, particular attention was paid to respiratory and ocular symptoms. All other observed clinical signs were recorded as well. Respiratory and ocular symptoms were scored as detailed in Table 8.

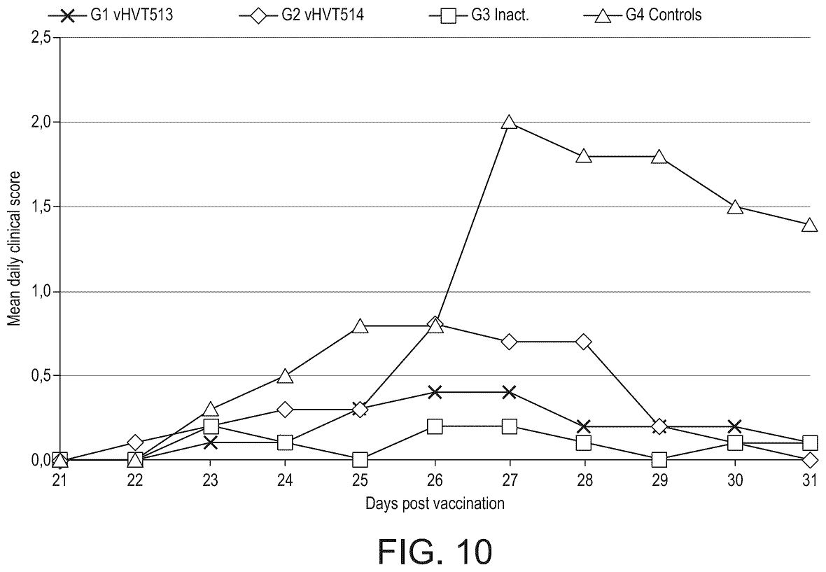

Results of clinical scoring are shown in FIG. 10 (Kinetic of mean clinical scores per group after H9N2 challenge). Two birds of control group (G4) died or were euthanized for ethical reasons on D27, 5 days post-challenge. The mean score of G1, G2, G3 and G4 was 2.0±2.5 (G1), 3.4±3.6 (G2), 1.0±2.5 (G3) and 10.9±11.2 (G4). All vaccines reduced the clinical signs.

### D. H9N2 Shedding

On D29 (7 days post vaccination), an oro-pharyngeal swabbing was carried out on all broiler chickens per group. The swabs were shipped to a laboratory for detection of the oro-pharyngeal H9N2 excretion by qRT-PCR.

(Mean oropharyngeal shedding evaluated by QRT-PCR): percentage of positive considering the limit of quantification (LOQ) of the PCR at 2.94 log 10. Results of G1 (vHVT513), G2 (vHVT514), G3 (G208 inactivated vaccine) and G4 (control) are shown in Table 11.

TABLE 11
Results of H2N9 shedding at 7 post
challenge (% positive (>2.94.LOD) qPCR:

| Groups | H9N2 shedding |
|---|---|
| G1 (vHVT513) | 50% |
| G2 (vHVT514) | 30% |
| G3 (Inact. H9) | 88% |
| G4 (control) | 90% |

Shedding levels and percentage of positive were significantly reduced in the vaccinated groups compared to the non-vaccinated control group as well as to group vaccinated with the conventional Inact. H9 vaccine.

### E. Conclusion

Recombinant HVT vaccines induced H9N2 antibody titers, decreased clinical signs and decreased shedding after challenge at D21 of broilers with H9N2, NDV and IBDV MDAs.

### Example 5: Protective Effects of vHVT513 Against IBD Challenge

### A. Objectives, Design of the Study, Vaccines and Vaccination

The objective of the study was to evaluate the IBD protection induced by vHVT513 in SPF chicks. The design of the experiment is shown in Table 12.

One-day-old SPF white leghorn (12/group except for 11 in group 4) were used in the study. They were vaccinated at D0 by the S.C. route with a target of 2500 PFU/bird of vHVT513 or left untreated.

TABLE 12
study design and protection results.

| Group | Vaccine | Dose | Challenge date | Protection |
|---|---|---|---|---|
| 1 | vHVT513 | 2500 PFU | D21 | 100% |
| 2 | — | — | D21 | 8.3% |
| 3 | vHVT513 | 2500 PFU | D28 | 100% |
| 4 | — | — | D28 | 0% |

### B. IBD Challenge and Protection

All chicks were challenged with the STC IBDV strain (2.0 log 10 EID50/0.03 mL dose) by eye drop route at D21 (groups 1 & 2) or at D28 (groups 3 & 4). Birds were observed for clinical signs for 4 days post-challenge. At D25 (groups 1 & 2) or at D32 (groups 3 & 4), they were euthanized, and bursa were examined for the presence of gross lesions due to the classical-STC IBDV. For purposes of this test, gross lesions included peri-bursal edema and/or edema and/or macroscopic hemorrhage in the bursal tissue, in accordance with 9 CFR 113.331 (c) (3). Any bird with these stated lesions were recorded as positive. Results of protection are shown in Table 12. All vaccinated birds were protected at both dates whereas most control birds were not.

### C. Conclusion

In conclusion, vHVT513 induced full protection against IBDV at 3 and 4 weeks post-vaccination.

### Example 6: Protective Effects of vHVT514 Against ND Challenge

### A. Objectives, Design of the Study, Vaccines and Vaccination

The objective of this study was to evaluate the efficacy of the recombinant virus vHVT514 in day-old SPF chicken after subcutaneous administration against velogenic Newcastle disease challenge. The secondary objective of this study was to assess the serological response induced by the candidate vaccine.

The study design is shown in Table 13. 33-day-old SPF chickens were allocated among two groups: 11 birds in G1 (controls) and 22 birds in G2 (vaccinated birds). Birds of G2 were vaccinated with 3.4 log 10 PFU of vHVT514.

TABLE 13
Study design and results of serology (mean titer in log 10 ±
standard deviation (number of positive/total) and ND protection.

| Group | Vaccine | NDV HI | Protection |
|---|---|---|---|
| G1 | — | 0.6 ± 0.0 | 0% |
| | | (0/10) | |
| G2 | vHVT514 | 1.08 ± 0.26 | 100% |
| | | (20/20) | |

### B. Serology

Blood sample was taken at D21 before challenge. Anti-NDV titers were evaluated by HI test using La Sota antigen. Results are summarized in Table 13. Negative values were set at the threshold value of 0.6 log 10 for NDV HI to calculate the mean and standard deviation. All vaccinated birds had NDV HI antibodies.

### C. ND Challenge and Protection

At D21, 10 control (G1) and 20 vaccinated (G2) chickens were challenged with a Malaysian (MAL04-01) strain of genotype VIId (or VII.1.1) by the intramuscular route with 0.2 mL containing 5.0 log 10 EID50. Protection was evaluated by looking at clinical signs during 2 weeks post-challenge.

All chickens in the control group G1 died within 3 days after challenge. None of the vaccinated chickens showed clinical signs or died after challenge.

### D. Conclusion

In conclusion, subcutaneous vaccination of day-old SPF chicks with a dose of 3.4 log 10 PFU/chick of vHVT514 induced 100% protection against an ND challenge using the MALAYSIAN MAL04-01 strain performed 21 days after vaccination.

SEQUENCE LISTING

SEQ ID NO: 1 (Amino Acid Sequence of Saudi Arabia codon optimized H9) (for vHVT513 and vHVT514)
MEPISLMIILLLVTTSNADKICIGHQSTNSTETVDTLTETNVPVTHAKELLHTEHNGMLCA
TNLGHPLILNTCTIEGLIYGNPSCDLLLGGREWSYIVERPSAVNGTCYPGNVENLEELRTL
FSSSSSYQRIQIFPDTIWNVTYTGTSKSCSDSFYRNMRWLTQKNGLYPVQDAQFINNRG
KDILFVWGIHHPPTDTAQTNLYTRTDTTTSVTTENLDRTFKPLIGPRPLVNGLIGRINYYW
SVLKPGQTLRVRSNGNLIAPWFGHVLSGESHGRILKTDLNSGNCVVQCQTEKGGLNSTL
PFHNISKYAFGTCPKYIGVKSLKLAIGLRNVPARSSRGLFGAIAGFIEGGWPGLVAGWYG
FQHSNDQGVGMAADRDSTQKAVDKITSKVNNIVDKMNKQYEIIDHEFSEVETRLNMIN
NKIDDQIQDVWAYNAELLVLLENQKTLDEHDANVNNLYNKVKRALGSNAMEDGKGCF
ELYHKCDDQCMETIRNGTYNRGKYKEESRLERQKIEGVKLESEGTYKILTIYSTVASSLV
LAMGLAAFLFWAMSNGSCRCNICI
SEQ ID NO: 2 (Amino acid sequence of a synthetic Infectious Bursal Disease Virus
structural capsid protein VP2) (for vHVT513)
MTNLQDQTQQIVPFIRSLLMPTTGPASIPDDTLEKHTLRSETSTYNLTVGDTGSGLIVFFP
GFPGSIVGAHYTLQSNGNYKFDQMLLTAQNLPASYNYCRLVSRSLTVRSSTLPGGVYAL
NGTINAVTFQGSLSELTDVSYNGLMSATANINDKIGNVLVGEGVTVLSLPTSYDLGYVR
LGDPIPAIGLDPKMVATCDSSDRPRVYTITAADDYQFSSQYQPGGVTITLESANIDAITSL
SIGGELVFQTSVQGLVLGATIYLIGFDGTAVITRAVAADNGLTAGTDNLMPFNLVIPTNEI
TQPITSIKLEIVTSKSGGQAGDQMSWSASGSLAVTIHGGNYPGALRPVTLVAYERVATGS
VVTVAGVSNFELIPNPELAKNLVTEYGRFDPGAMNYTKLILSERDRLGIKTVWPTREYT
DFREYFMEVADLNSPLKIAGAFGFKDIIRAIRR
SEQ ID NO: 3 (nucleotide sequence of pSaH9optIRESVP2 plasmid) (for vHVT513)
gacgaaagggcctcgtgatacgcctatttttataggttaatgtcatgataataatggtttcttagacgtcaggtggcacttttcggggaaatgtgc
gcggaacccctatttgtttatttttctaaatacattcaaatatgtatccgctcatgagacaataaccctgataaatgcttcaataatattgaaaaagg
aagagtatgagtattcaacatttccgtgtcgcccttattcccttttttgcggcattttgccttcctgtttttgctcacccagaaacgctggtgaaagt
aaaagatgctgaagatcagttgggtgcacgagtgggttacatcgaactggatctcaacagcggtaagatccttgagagttttcgccccgaag
aacgttttccaatgatgagcacttttaaagttctgctatgtggcgcggtattatcccgtattgacgccgggcaagagcaactcggtcgccgcat
acactattctcagaatgacttggttgagtactcaccagtcacagaaaagcatcttacggatggcatgacagtaagagaattatgcagtgctgc
cataaccatgagtgataacactgcggccaacttacttctgacaacgatcggaggaccgaaggagctaaccgcttttttgcacaacatggggg
atcatgtaactcgccttgatcgttgggaaccggagctgaatgaagccataccaaacgacgagcgtgacaccacgatgcctgtagcaatggc
aacaacgttgcgcaaactattaactggcgaactacttactctagcttcccggcaacaattaatagactggatggaggcggataaagttgcag
gaccacttctgcgctcggcccttccggctggctggtttattgctgataaatctggagccggtgagcgtgggtctcgcggtatcattgcagcac
tggggccagatggtaagccctcccgtatcgtagttatctacacgacggggagtcaggcaactatggatgaacgaaatagacagatcgctga
gataggtgcctcactgattaagcattggtaactgtcagaccaagtttactcatatatactttagattgatttaaaacttcatttttaatttaaaaggat
ctaggtgaagatcctttttgataatctcatgaccaaaatcccttaacgtgagttttcgttccactgagcgtcagaccccgtagaaaagatcaaag
gatcttcttgagatcctttttttctgcgcgtaatctgctgcttgcaaacaaaaaaaccaccgctaccagcggtggtttgtttgccggatcaagagc
taccaactctttttccgaaggtaactggcttcagcagagcgcagataccaaatactgttcttctagtgtagccgtagttaggccaccacttcaag
aactctgtagcaccgcctacatacctcgctctgctaatcctgttaccagtggctgctgccagtggcgataagtcgtgtcttaccgggttggact
caagacgatagttaccggataaggcgcagcggtcgggctgaacggggggttcgtgcacacagcccagcttggagcgaacgacctacac
cgaactgagatacctacagcgtgagctatgagaaagcgccacgcttcccgaagggagaaaggcggacaggtatccggtaagcggcagg
gtcggaacaggagagcgcacgagggagcttccagggggaaacgcctggtatctttatagtcctgtcgggtttcgccacctctgacttgagc
gtcgatttttgtgatgctcgtcaggggggcggagcctatggaaaaacgccagcaacgcggcctttttacggttcctggccttttgctggcctttt
gctcacatgttctttcctgcgttatcccctgattctgtggataaccgtattaccgcctttgagtgagctgataccgctcgccgcagccgaacgac
cgagcgcagcgagtcagtgagcgaggaagcggaagagcgcccaatacgcaaaccgcctctccccgcgcgttggccgattcattaatgc
agctggcacgacaggtttcccgactggaaagcgggcagtgagcgcaacgcaattaatgtgagttagctcactcattaggcaccccaggctt
tacactttatgcttccggctcgtatgttgtgtggaattgtgagcggataacaatttcacacaggaaacagctatgaccatgattacgccgcggc
cgctctagaactcgtcgatcgcagcgatgacaaacctgcaagatcaaacccaacagattgttccgttcatacggagccttctgatgccaaca
accggaccggcgtccattccggacgacaccctggagaagcacactctcaggtcagagacctcgacctacaatttgactgtgggggacaca
gggtcagggctaattgtctttttccctggattccctggctcaattgtgggtgctcactacacactgcagagcaatgggaactacaagttcgatc
agatgctcctgactgcccagaacctaccggccagctacaactactgcagactagtgagtcggagtctcacagtgaggtcaagcacactccc
tggtggcgtttatgcactaaacggcaccataaacgccgtgaccttccaaggaagcctgagtgaactgacagatgttagctacaatgggttga
tgtctgcaacagccaacatcaacgacaaaattgggaatgtcctggtaggggaaggggtcactgtcctcagcctacccacatcatatgatctt
gggtatgtgaggcttggtgaccccattcccgctatagggcttgacccaaaaatggtagctacatgcgacagcagtgacaggcccagagtct
acaccataactgcagccgatgattaccaattctcatcacagtaccaaccaggggggtaacaatcacactgttctcagccaacattgatgctat
cacaagcctcagcattgggggagagctcgtgtttcaaacaagcgtccaaggccttgtactgggcgccaccatctaccttataggctttgatg
ggactgcggtaatcaccagagctgtagccgcagataatgggctgacggccggcaccgacaatcttatgccattcaatcttgtcattccaacc
aatgagataacccagccaatcacatccatcaaactggagatagtgacctccaaaagtggtggtcaggcaggggatcagatgtcatggtcgg
caagtgggagcctagcagtgacgatccatggtggcaactatccaggggccctccgtcccgtcacactagtagcctacgaaagagtggcaa
caggatccgtcgttacggtcgctggggtgagtaacttcgagctgattccaaatcctgaactagcaaagaacctggttacagaatacggccga
tttgacccaggagccatgaactacacaaaattgatactgagtgagagggaccgtcttggcatcaagaccgtctggccaacaagggagtaca
ctgattttcgtgagtacttcatggaggtggccgacctcaactctcccctgaagattgcaggagcatttggcttcaaagacataatccgggctat
aaggaggtaacccccccccctaacgttactggccgaagccgcttggaataaggccggtgtgcgtttgtctatatgttattttccaccatattgc
cgtcttttggcaatgtgagggcccggaaacctggccctgtcttcttgacgagcattcctaggggtctttcccctctcgccaaaggaatgcaag
gtctgttgaatgtcgtgaaggaagcagttcctctggaagcttcttgaagacaaacaacgtctgtagcgaccctttgcaggcagcggaacccc
ccacctggcgacaggtgcctctgcggccaaaagccacgtgtataagatacacctgcaaaggcggcacaaccccagtgccacgttgtgagt
tggatagttgtggaaagagtcaaatggctctcctcaagcgtattcaacaaggggctgaaggatgcccagaaggtaccccattgtatgggatc
tgatctggggcctcggtgcacatgctttacatgtgtttagtcgaggttaaaaaacgtctaggccccccgaaccacggggacgtggttttccttt
gaaaaacacgatgataataccatggaaccaattagcctgatgattatcctgctgctggttaccacctctaacgctgacaaaatctgcatcggtc
accagtctaccaactctaccgaaaccgttgacaccctgaccgaaaccaacgttccggttacccacgctaaagaactgctgcacaccgaaca
caacggtatgctgtgcgctaccaacctgggtcacccgctgatcctgaacacctgcactattgaaggtctgatctacggtaacccgtcttgcga
cctgctgctgggtggtcgtgaatggtcttacatcgttgaacgtccgtctgctgttaacggtacctgctacccgggtaacgttgaaaacctggaa
gaactgcgtaccctgttctcttcttcttcttcttaccagcgtatccagatcttcccggacaccatctggaacgttacctacaccggtacctctaaat
cttgctctgactctttctaccgtaacatgcgttggctgacccagaaaaacggtctgtacccggttcaggacgctcagttcaccaacaaccgtg
gtaaagacatcctgttcgtttggggtatccaccacccgccgaccgacaccgctcagaccaacctgtacacccgtaccgacaccaccacctc
tgttaccaccgaaaacctggaccgtaccttcaaaccgctgatcggtccgcgtccgctggttaacggtctgatcggtcgtatcaactactactg
gtctgttctgaaaccgggtcagaccctgcgtgttcgttctaacggtaacctgatcgctccgtggttcggtcacgttctgtctggtgaatctcacg
gtcgtatcctgaaaaccgatctgaactctggtaactgcgttgttcagtgccagaccgaaaaaggtggtctgaactctaccctgccgttccaca
acatctctaaatacgctttcggtacttgcccgaaatacatcggtgttaaatctctgaaactggctatcggtctgcgtaacgttccggctaggtctt
cccgtggtctgttcggtgctatcgctggtttcatcgaaggtggttggccgggtctggttgctggttggtacggtttccagcactctaacgacca
gggtgttggtatggctgctgaccgtgattctacccagaaagctgttgacaaaatcacctctaaagttaacaacatcgttgacaaaatgaacaa
acagtacgaaatcatcgaccacgaattctctgaagttgaaacccgtctgaacatgatcaacaacaaaatcgacgaccagatccaggacgttt
gggcttacaacgctgaactgctggttctgctggaaaaccagaaaaccctggacgaacacgacgctaacgttaacaacctgtacaacaaagt
taaacgtgctctgggttctaacgctatggaagacggtaaaggttgcttcgaactgtaccacaaatgcgacgaccagtgcatggaaaccatcc
gtaacggtacctacaaccgtgggaaatacaaagaagaatctcgtctggaacgtcagaaaatcgaaggtgttaaactggaatctgaaggtac
ctacaaaatcctgaccatctactctaccgttgcttcttctctggttctggctatgggtctggctgctttcctgttctgggctatgtctaacggttcttg
ccgttgcaacatctgcatctaatgaccgcggggatccagacatgataagatacattgatgagtttggacaaaccacaactagaatgcagtga
aaaaaatgctttatttgtgaaatttgtgatgctattgctttatttgtaaccattataagctgcaataaacaagttaacaacaacaattgcattcatttta
tgtttcaggttcagggggaggtgtgggaggttttttcggatcctctagacctgcagggtcgacaattattttatttaataacatatagcccaaaga
cctctatgaacatttagtttcccgtatactcaacggcgcgtgtacacacgcatctctttgcatagcgatgaagtttgttcggcagcagaaaatgc
agatatccaacaatctggagaaaacttatcatcacagtggcagtggaaacataccccctctatattcatggtataattatcgtctacagcgtcca
ggatagtggcgtgagaaaatggagatctgcagccctcctttccatggcatgccgctttattgttcattaaacgcacaatggtctcaacgccag
atatgggcatagattctgaagaacccgttgacaatccgaagaagaaggcgtgcaggtctttggaagactcgcacgttggtcttataatgtatg
atcgagatgtcaccctaatgccacatggtacaggcttatcgcggtcatggcgatcggacttgtaatttgcaacgatgggcaaaggatcgacg
acatgccaaacattctgaacccgtagagatgttaacgatgacgaggatgaatatcccatgctcgctgccatagtatcaagtacaccgcgaat
aaggacgcgtccaacatcgttatatgcacacaatgggctacacgtgactaacacccccgaatattagtcatatgtgagtttcagtctggctccc
atatagcctgtagactatttgtggtttaagtgtgaacgaggcgctgtgaacgagactcgggccgattgtaagaacaagcaagaattcactggc
cgtcgttttacaacgtcgtgactgggaaaaccctggcgttacccaacttaatcgccttgcagcacatccccctttcgccagctggcgtaatag
cgaagaggcccgcaccgatcgcccttcccaacagttgcgcagcctgaatggcgaatggcgcctgatgcggtattttctccttacgcatctgt
gcggtatttcacaccgcatatggtgcactctcagtacaatctgctctgatgccgcatagttaagccagccccgacacccgccaacacccgct
gacgcgccctgacgggcttgtctgctcccggcatccgcttacagacaagctgtgaccgtctccgggagctgcatgtgtcagaggttttcacc
gtcatcaccgaaacgcgcga
SEQ ID NO: 4 (vHVT513 nucleotide sequence) (vHVT513)
accgccagatatccaaagtacagcacgatgttgcacggacgactttgcagtcaccagccttcctttccacccccccaccaacaaaatgtttat
cgtaggacccatatccgtaataaggatgggtctggcagcaaccccataggcgcctcggcgtggtagttctcgaggatacatccaaagaggt
tgagtattctctctacacttcttgttaaatggaaagtgcatttgcttgttcttacaatcggcccgagtctcgttcacagcgcctcgttcacacttaaa
ccacaaatagtctacaggctatatgggagccagactgaaactcacatatgactaatattcgggggtgttagtcacgtgtagcccattgtgtgc
atataacgatgttggacgcgtccttattcgcggtgtacttgatactatggcagcgagcatgggatattcatcctcgtcatcgttaacatctctacg
ggttcagaatgtttggcatgtcgtcgatcctttgcccatcgttgcaaattacaagtccgatcgccatgaccgcgataagcctgtaccatgtggc
attagggtgacatctcgatcatacattataagaccaacgtgcgagtcttccaaagacctgcacgccttcttcttcggattgtcaacgggttcttc
agaatctatgcccatatctggcgttgagaccattgtgcgtttaatgaacaataaagcggcatgccatggaaaggagggctgcagatctccatt
ttctcacgccactatcctggacgctgtagacgataattataccatgaatatagagggggtatgtttccactgccactgtgatgataagttttctcc
agattgttggatatctgcattttctgctgccgaacaaacttcatcgctatgcaaagagatgcgtgtgtacacgcgccgttgagtatacgggaaa
ctaaatgttcatagaggtctttgggctatatgttattaaataaaataattgtcgaccctgcaggtctagaggatccgaaaaaacctcccacacct
ccccctgaacctgaaacataaaatgaatgcaattgttgttgttaacttgtttattgcagcttataatggttacaaataaagcaatagcatcacaaat
ttcacaaataaagcatttttttcactgcattctagttgtggtttgtccaaactcatcaatgtatcttatcatgtctggatccccgcggtcattagatgc
agatgttgcaacggcaagaaccgttagacatagcccagaacaggaaagcagccagacccatagccagaaccagagaagaagcaacgg
tagagtagatggtcaggattttgtaggtaccttcagattccagtttaacaccttcgattttctgacgttccagacgagattcttctttgtatttcccac
ggttgtaggtaccgttacggatggtttccatgcactggtcgtcgcatttgtggtacagttcgaagcaacctttaccgtcttccatagcgttagaa
cccagagcacgtttaactttgttgtacaggttgttaacgttagcgtcgtgttcgtccagggttttctggttttccagcagaaccagcagttcagcg
ttgtaagcccaaacgtcctggatctggtcgtcgattttgttgttgatcatgttcagacgggtttcaacttcagagaattcgtggtcgatgatttcgt
actgtttgttcattttgtcaacgatgttgttaactttagaggtgattttgtcaacagctttctgggtagaatcacggtcagcagccataccaacacc
ctggtcgttagagtgctggaaaccgtaccaaccagcaaccagacccggccaaccaccttcgatgaaaccagcgatagcaccgaacagac
cacgggaagacctagccggaacgttacgcagaccgatagccagtttcagagatttaacaccgatgtatttcgggcaagtaccgaaagcgta
tttagagatgttgtggaacggcagggtagagttcagaccacctttttcggtctggcactgaacaacgcagttaccagagttcagatcggttttc
aggatacgaccgtgagattcaccagacagaacgtgaccgaaccacggagcgatcaggttaccgttagaacgaacacgcagggtctgacc
cggtttcagaacagaccagtagtagttgatacgaccgatcagaccgttaaccagcggacgcggaccgatcagcggtttgaaggtacggtc
caggttttcggtggtaacagaggtggtggtgtcggtacgggtgtacaggttggtctgagcggtgtcggtcggcgggtggtggataccccaa
acgaacaggatgtctttaccacggttgttggtgaactgagcgtcctgaaccgggtacagaccgtttttctgggtcagccaacgcatgttacgg
tagaaagagtcagagcaagatttagaggtaccggtgtaggtaacgttccagatggtgtccgggaagatctggatacgctggtaagaagaag
aagaagagaacagggtacgcagttcttccaggttttcaacgttacccgggtagcaggtaccgttaacagcagacggacgttcaacgatgta
agaccattcacgaccacccagcagcaggtcgcaagacgggttaccgtagatcagaccttcaatagtgcaggtgttcaggatcagcgggtg
acccaggttggtagcgcacagcataccgttgtgttcggtgtgcagcagttctttagcgtgggtaaccggaacgttggtttcggtcagggtgtc
aacggtttcggtagagttggtagactggtgaccgatgcagattttgtcagcgttagaggtggtaaccagcagcaggataatcatcaggctaat
tggttccatggtattatcatcgtgtttttcaaaggaaaaccacgtccccgtggttcggggggcctagacgttttttaacctcgactaaacacatgt
aaagcatgtgcaccgaggccccagatcagatcccatacaatggggtaccttctgggcatccttcagccccttgttgaatacgcttgaggaga
gccatttgactctttccacaactatccaactcacaacgtggcactggggttgtgccgcctttgcaggtgtatcttatacacgtggcttttggccg
cagaggcacctgtcgccaggtggggggttccgctgcctgcaaagggtcgctacagacgttgtttgtcttcaagaagcttccagaggaactg
cttccttcacgacattcaacagaccttgcattcctttggcgagaggggaaagacccctaggaatgctcgtcaagaagacagggccaggtttc
cgggccctcacattgccaaaagacggcaatatggtggaaaataacatatagacaaacgcacaccggccttattccaagcggcttcggcca
gtaacgttaggggggggggttacctccttatagcccggattatgtctttgaagccaaatgctcctgcaatcttcaggggagagttgaggtcgg
ccacctccatgaagtactcacgaaaatcagtgtactcccttgttggccagacggtcttgatgccaagacggtccctctcactcagtatcaatttt
gtgtagttcatggctcctgggtcaaatcggccgtattctgtaaccaggttctttgctagttcaggatttggaatcagctcgaagttactcacccca
gcgaccgtaacgacggatcctgttgccactctttcgtaggctactagtgtgacgggacggagggcccctggatagttgccaccatggatcgt
cactgctaggctcccacttgccgaccatgacatctgatcccctgcctgaccaccacttttggaggtcactatctccagtttgatggatgtgattg
gctgggttatctcattggttggaatgacaagattgaatggcataagattgtcggtgccggccgtcagcccattatctgcggctacagctctggt
gattaccgcagtcccatcaaagcctataaggtagatggtggcgcccagtacaaggccttggacgcttgtttgaaacacgagctctcccccaa
tgctgaggcttgtgatagcatcaatgttggctgagaacagtgtgattgttaccccacctggttggtactgtgatgagaattggtaatcatcggct
gcagttatggtgtagactctgggcctgtcactgctgtcgcatgtagctaccatttttgggtcaagccctatagcgggaatggggtcaccaagc
ctcacatacccaagatcatatgatgtgggtaggctgaggacagtgaccccttcccctaccaggacattcccaattttgtcgttgatgttggctgt
tgcagacatcaacccattgtagctaacatctgtcagttcactcaggcttccttggaaggtcacggcgtttatggtgccgtttagtgcataaacgc
caccagggagtgtgcttgacctcactgtgagactccgactcactagtctgcagtagttgtagctggccggtaggttctgggcagtcaggagc
atctgatcgaacttgtagttcccattgctctgcagtgtgtagtgagcacccacaattgagccagggaatccagggaaaaagacaattagccct
gaccctgtgtcccccacagtcaaattgtaggtcgaggtctctgacctgagagtgtgcttctccagggtgtcgtccggaatggacgccggtcc
ggttgttggcatcagaaggctccgtatgaacggaacaatctgttgggtttgatcttgcaggtttgtcatcgctgcgatcgacgagttctagagc
ggccgcctgcagcgaggagctctgcgttctacggtggtcagaccgaagactgcgacggtaccgacgctggtcgcgcctcttatacccacg
tagaacgcagctcagccaatagaatgcgtgccaatatggaatttccaggggaaaaccggggcggtgttacgttttggctgccctttcacttcc
cattgacgtgtattggctcgagaacggtactttcccattaatcagctatgggaaagtaccgtttaaaggtcacgttgcattagtttcaatagccca
ttgacgtcaatggtgggaaagtacatggcgttttaattaaattggctggaaaaacccaatgactcacccctattgaccttatgtacgtgccaata
atgggaaaaacccattgactcaccccctattgaccttttgtactgggcaaaacccaatggaaagtccctattgactcagtgtacttggctccaat
gggactttcctgttgattcacccctattgaccttatgtactgggcaaaacccattggaaagtccctaatgactcagtatacgtgccagtaatggg
aaaaacccattggcttacctcccattgaccttatgtactgggcaaaacccattggaaagtccctattgactcaatgtacttggctccaatgggac
tttcctgttgactcaccccctattgaccttatgtactgggcaaaacccaatggaaagtccctattgagtcagtgtacttggctccaatgggtttttc
ccattgactcatcccctattgaccttatgtactgggcaaaacccaatggaaagtccctattgacgcagtgtacttggctccaatgggactttcct
gttgattcaccccctattgaccttatgtactgggcagaatacaatggaaagtccctattgactcacccacattgaccttatatgcttgccaacaat
ggaaaaacccattggaaagtccctattgagtcagtgtacttggcagcaatgggtttttcccattggctcacctcccattgacccaatgtacttgg
gcaaaacccagtggaaagtcccatttgactcagtgtgcttgccagtaatgggaaaaacccattggcttacctcccattgacccaatgtacttg
ggtaaaggccattgagtcaccacccctatgctgggaaatggtgaacgccccctatgtggaaagtccctatgggccgccattagagtgcatg
accgtgctgattcatatgccatatgagtgtattagggggctttccgcttgggaaattgggtaaaaagtccccgtattactcacatagggggcgt
ttggctttgcaaattaggggatttcagtgcatttggcattaaaaactattggttctagtcataaaacgggcggagttgggggatccactagtgaa
ttcgtttaatgttagtttattcaatgcattggttgcaaatattcattacttctccaatcccaggtcattctttagcgagatgatgttatgacattgctgtg
aaaattactacaggatatatttttaagatgcaggagtaacaatgtgcatagtaggcgtagttatcgcagacgtgcaacgcttcgcatttgagtta
ccgaagtgcccaacagtgctgcggttatggtttatgcgcacagaatccatgcatgtcctaattgaaccatccgatttttcttttaatcgcgatcgt
tgtttgggcaactgcgttatttcagatctaaaaaatttaccctttatgaccatcacatctctctggctcataccccgcttggataagatatcatgtag
attccgccctaagaaatgcaaactaacattattgtcggttccatatacacttccatcttgtccttcgaaaataacaaactcgcgcaatagaccgtc
cgtacatgcatggccgatgtgtgtcaacatcattggtctgctagatcccgatgggacgaatcgtacagtcgtcgctccagcattggcaaaaat
ccccagataccctccatgcggcaaatctaaattgcgaccccgaagagactgcaccaaagtcttatcgacgcacgctgatttttttgaacagcg
ggagcccattatcttcagtggagcgtagacgggcgaggctaattatgtgacatagcaacactgcatgtatgtttttataaatcaataagagtac
ataatttattacgtatcatttccgtttgtaatatactgtatacatcatccacactattagtcagcactagcgcgcgggcgcacgttacaatagcagc
gtgcccgttatctatattgtccgatatttacacataacatttcatcgacatgattaaatacctaagtactgcacacagatgtttaatgtatatcgtcat
ataaattatatcgctaggacagacccaaacgacctttatcccaaacagtcagatcctcttctcaagtgtcgatttctgttatggaatatgcatacc
ctggcccagaaattgcacgcacgagcgtagtgaatgcgtcattggttttacatttaaaggctaaatgcacaaattctttagacgacagcacatc
gttaaatagcatctctagcgttcttatgaatgctaagcattggagtcctcctggtcggccacaataacagctgagtatcataccctgagctccg
gggttgtcgcacatagcggattcgtataaacataggattttccgcgaatccatcagttgcaaaaatctgttaggctccatcaacaacgctggat
ttacttcagatccacgcgtaaagtaatggtgctcgaataccgtttttagagttgtcggcatttcaaggaacaaaga
SEQ ID NO: 5 (Amino acid sequence of NDV VIId wt) (for vHVT514)
MGSKPSTRIPAPLMLITRIMLILGCIRPTSSLDGRPLAAAGIVVTGDKA VNVYTSSQTGSII
VKLLPNMPRDKEACAKAPLEAYNRTLTTLLTPLGDSIRKIQGSVSTSGGGKQGRLIGAVI
GSVALGVATAAQITAAAALIQANQNAANILRLKESIAATNEAVHEVTDGLSQLSVAVGK
MQQFVNDQFNNTARELDCIKITQQVGVELNLYLTELTTVFGPQITSPALTQLTIQALYNL
AGGNMDYLLTKLGIGNNQLSSLIGSGLITGYPILYDSQTQLLGIQVNLPSVGNLNNMRAT
YLETLSVSTTKGYASALVPKVVTQVGSVIEELDTSYCIESDLDLYCTRIVTFPMSPGIYSC
LSGNTSACMYSKTEGALTTPYMALKGSVIANCKITTCRCTDPPGIISQNYGEAVSLIDRH
SCNVLSLDGITLRLSGEFDATYQKNISILDSQVIVTGNLDISTELGNVNNSISNALDRLAES
NSKLEKVNVRLTSTSALITYIVLTVISLVFGALSLVLACYLMYKQKAQQKTLLWLGNNTLDQMRATTRA
SEQ ID NO: 6 (nucleotide sequence of pFwtIRESSaH9op plasmid) (vHVT514)
ctaaattgtaagcgttaatattttgttaaaattcgcgttaaatttttgttaaatcagctcattttttaaccaataggccgaaatcggcaaaatcccttat
aaatcaaaagaatagaccgagatagggttgagtgttgttccagtttggaacaagagtccactattaaagaacgtggactccaacgtcaaagg
gcgaaaaaccgtctatcagggcgatggcccactacgtgaaccatcaccctaatcaagttttttggggtcgaggtgccgtaaagcactaaatc
ggaaccctaaagggagcccccgatttagagcttgacggggaaagccggcgaacgtggcgagaaaggaagggaagaaagcgaaagga
gcgggcgctagggcgctggcaagtgtagcggtcacgctgcgcgtaaccaccacacccgccgcgcttaatgcgccgctacagggcgcgt
cccattcgccattcaggctgcgcaactgttgggaagggcgatcggtgcgggcctcttcgctattacgccagctggcgaaagggggatgtg
ctgcaaggcgattaagttgggtaacgccagggttttcccagtcacgacgttgtaaaacgacggccagtgagcgcgcgtaatacgactcact
atagggcgaattggagctcagggtatgatactcagctgttattgtggccgaccaggaggactccaatgcttagcattcataagaacgctaga
gatgctatttaacgatgtgctgtcgtctaaagaatttgtgcatttagcctttaaatgtaaaaccaatgacgcattcactacgctcgtgcgtgcaatt
tctgggccagggtatgcatattccataacagaaatcgacacttgagaagaggatctgactgtttgggataaaggtcgtttgggtctgtcctagc
gatataatttatatgacgatatacattaaacatctgtgtgcagtacttaggtatttaatcatgtcgatgaaatgttatgtgtaaatatcggacaatata
gataacgggcacgctgctattgtaacgtgcgcccgcgcgctagtgctgactaatagtgtggatgatgtatacagtatattacaaacggaaatg
atacgtaataaattatgtactcttattgatttataaaaacatacatgcagtgttgctatgtcacataattagcctcgcccgtctacgctccactgaag
ataatgggctcccgctgttcaaaaaaatcagcgtgcgtcgataagactttggtgcagtctcttcggggtcgcaatttagatttgccgcatggag
ggtatctggggatttttgccaatgctggagcgacgactgtacgattcgtcccatcgggatctagcagaccaatgatgttgacacacatcggcc
atgcatgtacggacggtctattgcgcgagtttgttattttcgaaggacaagatggaagtgtatatggaaccgacaataatgttagtttgcatttctt
agggcggaatctacatgatatcttatccaagcggggtatgagccagagagatgtgatggtcataaagggtaaattttttagatctgaaataac
gcagttgcccaaacaacgatcgcgattaaaagaaaaatcggatggttcaattaggacatgcatggattctgtgcgcataaaccataaccgca
gcactgttgggcacttcggtaactcaaatgcgaagcgttgcacgtctgcgataactacgcctactatgcacattgttactcctgcatcttaaaaa
tatatcctgtagtaattttcacagcaatgtcataacatcatctcgctaaagaatgacctgggattggagaagtaatgaatatttgcaaccaatgca
ttgaataaactaacattaaacgaattcactagtggatcccccaactccgcccgttttatgactagaaccaatagtttttaatgccaaatgcactga
aatcccctaatttgcaaagccaaacgccccctatgtgagtaatacggggactttttacccaatttcccaagcggaaagccccctaatacactc
atatggcatatgaatcagcacggtcatgcactctaatggcggcccatagggactttccacatagggggcgttcaccatttcccagcataggg
gtggtgactcaatggcctttacccaagtacattgggtcaatgggaggtaagccaatgggtttttcccattactggcaagcacactgagtcaaat
gggactttccactgggttttgcccaagtacattgggtcaatgggaggtgagccaatgggaaaaacccattgctgccaagtacactgactcaa
tagggactttccaatgggtttttccattgttggcaagcatataaggtcaatgtgggtgagtcaatagggactttccattgtattctgcccagtacat
aaggtcaatagggggtgaatcaacaggaaagtcccattggagccaagtacactgcgtcaatagggactttccattgggttttgcccagtaca
taaggtcaataggggatgagtcaatgggaaaaacccattggagccaagtacactgactcaatagggactttccattgggttttgcccagtaca
taaggtcaatagggggtgagtcaacaggaaagtcccattggagccaagtacattgagtcaatagggactttccaatgggttttgcccagtac
ataaggtcaatgggaggtaagccaatgggtttttcccattactggcacgtatactgagtcattagggactttccaatgggttttgcccagtacat
aaggtcaataggggtgaatcaacaggaaagtcccattggagccaagtacactgagtcaatagggactttccattgggttttgcccagtacaa
aaggtcaatagggggtgagtcaatgggtttttcccattattggcacgtacataaggtcaataggggtgagtcattgggtttttccagccaattta
attaaaacgccatgtactttcccaccattgacgtcaatgggctattgaaactaatgcaacgtgacctttaaacggtactttcccatagctgattaa
tgggaaagtaccgttctcgagccaatacacgtcaatgggaagtgaaagggcagccaaaacgtaacaccgccccggttttcccctggaaatt
ccatattggcacgcattctattggctgagctgcgttctacgtgggtataagaggcgcgaccagcgtcggtaccgtcgcagtcttcggtctgac
caccgtagaacgcagagctcctcgctgcaggcggccgcatgggctccaaaccttctaccaggatcccagcacctctgatgctgatcaccc
ggattatgctgatattgggctgtatccgtccgacaagctctcttgacggcaggcctcttgcagctgcaggaattgtagtaacaggagataagg
cagtcaatgtatacacttcgtctcagacagggtcaatcatagtcaagttgctcccgaatatgcccagggataaggaggcgtgtgcaaaagcc
ccattagaggcatataacagaacactgactactttgctcactcctcttggcgactccatccgcaagatccaagggtctgtgtccacatctggag
gaggcaagcaaggccgcctgataggtgctgttattggcagtgtagctcttggggttgcaacagcggcacagataacagcagctgcggccc
taatacaagccaaccagaatgccgccaacatcctccggcttaaggagagcattgctgcaaccaatgaagctgtgcatgaagtcaccgacg
gattatcacaactatcagtggcagttgggaagatgcagcagtttgtcaatgaccagtttaataatacggcgcgagaattggactgtataaaaat
cacacaacaggttggtgtagaactcaacctatacctaactgaattgactacagtattcgggccacagatcacctcccctgcattaactcagctg
accatccaggcactttataatttagctggtggcaatatggattacttattaactaagttaggtatagggaacaatcaactcagctcgttaattggta
gcggcctgatcactggttaccctatactgtatgactcacagactcaactcttgggcatacaagtgaatttaccctcagtcgggaacttaaataat
atgcgtgccacctatttggagaccttatctgtaagtacaaccaaaggatatgcctcagcacttgtcccgaaagtagtgacacaagtcggttcc
gtgatagaagagcttgacacctcatactgtatagagtccgatctggatttatattgtactagaatagtgacattccccatgtccccaggtatttatt
cctgtttgagcggcaacacatcagcttgcatgtattcaaagactgaaggcgcactcactacgccgtatatggcccttaaaggctcagttattgc
caattgtaaaataacaacatgtagatgtacagaccctcctggtatcatatcgcaaaattatggagaagctgtatccctgatagatagacattcgt
gcaatgtcttatcattagacgggataactctaaggctcagtggggaatttgatgcaacttatcaaaagaacatctcaatactagattctcaagtc
atcgtgacaggcaatcttgatatatcaactgaacttggaaacgtcaacaattcaatcagcaatgccttggataggttggcagaaagcaacagc
aagctagaaaaagtcaatgtcagactaaccagcacatctgctctcattacctatattgttctaactgtcatttctctagttttcggtgcacttagtct
ggtgttagcgtgttacctgatgtacaaacagaaggcacaacaaaagaccttgctatggcttgggaataataccctcgatcagatgagagcca
ctacaagagcatgagcggccgccccccccccccctaacgttactggccgaagccgcttggaataaggccggtgtgcgtttgtctatatgtta
ttttccaccatattgccgtcttttggcaatgtgagggcccggaaacctggccctgtcttcttgacgagcattcctaggggtctttcccctctcgcc
aaaggaatgcaaggtctgttgaatgtcgtgaaggaagcagttcctctggaagcttcttgaagacaaacaacgtctgtagcgaccctttgcag
gcagcggaaccccccacctggcgacaggtgcctctgcggccaaaagccacgtgtataagatacacctgcaaaggcggcacaaccccag
tgccacgttgtgagttggatagttgtggaaagagtcaaatggctctcctcaagcgtattcaacaaggggctgaaggatgcccagaaggtacc
ccattgtatgggatctgatctggggcctcggtgcacatgctttacatgtgtttagtcgaggttaaaaaacgtctaggccccccgaaccacggg
gacgtggttttcctttgaaaaacacgatgataataccatggaaccaattagcctgatgattatcctgctgctggttaccacctctaacgctgaca
aaatctgcatcggtcaccagtctaccaactctaccgaaaccgttgacaccctgaccgaaaccaacgttccggttacccacgctaaagaactg
ctgcacaccgaacacaacggtatgctgtgcgctaccaacctgggtcacccgctgatcctgaacacctgcactattgaaggtctgatctacgg
taacccgtcttgcgacctgctgctgggtggtcgtgaatggtcttacatcgttgaacgtccgtctgctgttaacggtacctgctacccgggtaac
gttgaaaacctggaagaactgcgtaccctgttctcttcttcttcttcttaccagcgtatccagatcttcccggacaccatctggaacgttacctac
accggtacctctaaatcttgctctgactctttctaccgtaacatgcgttggctgacccagaaaaacggtctgtacccggttcaggacgctcagtt
caccaacaaccgtggtaaagacatcctgttcgtttggggtatccaccacccgccgaccgacaccgctcagaccaacctgtacacccgtacc
gacaccaccacctctgttaccaccgaaaacctggaccgtaccttcaaaccgctgatcggtccgcgtccgctggttaacggtctgatcggtcg
tatcaactactactggtctgttctgaaaccgggtcagaccctgcgtgttcgttctaacggtaacctgatcgctccgtggttcggtcacgttctgtc
tggtgaatctcacggtcgtatcctgaaaaccgatctgaactctggtaactgcgttgttcagtgccagaccgaaaaaggtggtctgaactctac
cctgccgttccacaacatctctaaatacgctttcggtacttgcccgaaatacatcggtgttaaatctctgaaactggctatcggtctgcgtaacgt
tccggctaggtcttcccgtggtctgttcggtgctatcgctggtttcatcgaaggtggttggccgggtctggttgctggttggtacggtttccagc
actctaacgaccagggtgttggtatggctgctgaccgtgattctacccagaaagctgttgacaaaatcacctctaaagttaacaacatcgttga
caaaatgaacaaacagtacgaaatcatcgaccacgaattctctgaagttgaaacccgtctgaacatgatcaacaacaaaatcgacgaccag
atccaggacgtttgggcttacaacgctgaactgctggttctgctggaaaaccagaaaaccctggacgaacacgacgctaacgttaacaacc
tgtacaacaaagttaaacgtgctctgggttctaacgctatggaagacggtaaaggttgcttcgaactgtaccacaaatgcgacgaccagtgc
atggaaaccatccgtaacggtacctacaaccgtgggaaatacaaagaagaatctcgtctggaacgtcagaaaatcgaaggtgttaaactgg
aatctgaaggtacctacaaaatcctgaccatctactctaccgttgcttcttctctggttctggctatgggtctggctgctttcctgttctgggctatg
tctaacggttcttgccgttgcaacatctgcatctaatgaccgcggggatccagacatgataagatacattgatgagtttggacaaaccacaact
agaatgcagtgaaaaaaatgctttatttgtgaaatttgtgatgctattgctttatttgtaaccattataagctgcaataaacaagttaacaacaaca
attgcattcattttatgtttcaggttcagggggaggtgtgggaggttttttcggatcctctagagtcgacaattattttatttaataacatatagccca
aagacctctatgaacatttagtttcccgtatactcaacggcgcgtgtacacacgcatctctttgcatagcgatgaagtttgttcggcagcagaaa
atgcagatatccaacaatctggagaaaacttatcatcacagtggcagtggaaacataccccctctatattcatggtataattatcgtctacagcg
tccaggatagtggcgtgagaaaatggagatctgcagccctcctttccatggcatgccgctttattgttcattaaacgcacaatggtctcaacgc
cagatatgggcatagattctgaagaacccgttgacaatccgaagaagaaggcgtgcaggtctttggaagactcgcacgttggtcttataatgt
atgatcgagatgtcaccctaatgccacatggtacaggcttatcgcggtcatggcgatcggacttgtaatttgcaacgatgggcaaaggatcg
acgacatgccaaacattctgaacccgtagagatgttaacgatgacgaggatgaatatcccatgctcgctgccatagtatcaagtacaccgcg
aataaggacgcgtccaacatcgttatatgcacacaatgggctacacgtgactaacacccccgaatattagtcatatgtgagtttcagtctggct
cccatatagcctgtagactatttgtggtttaagtgtgaacgaggcgctgtgaacgagactcgggccgattgtaagaacaagcaaatgcacttt
ccatttaacaagaagtgtagagagaatactcaacctctttggatgtatcctcgagggggggcccggtacccagcttttgttccctttagtgagg
gttaattgcgcgcttggcgtaatcatggtcatagctgtttcctgtgtgaaattgttatccgctcacaattccacacaacatacgagccggaagca
taaagtgtaaagcctggggtgcctaatgagtgagctaactcacattaattgcgttgcgctcactgcccgctttccagtcgggaaacctgtcgtg
ccagctgcattaatgaatcggccaacgcgcggggagaggcggtttgcgtattgggcgctcttccgcttcctcgctcactgactcgctgcgct
cggtcgttcggctgcggcgagcggtatcagctcactcaaaggcggtaatacggttatccacagaatcaggggataacgcaggaaagaac
atgtgagcaaaaggccagcaaaaggccaggaaccgtaaaaaggccgcgttgctggcgtttttccataggctccgcccccctgacgagcat
cacaaaaatcgacgctcaagtcagaggtggcgaaacccgacaggactataaagataccaggcgtttccccctggaagctccctcgtgcgc
tctcctgttccgaccctgccgcttaccggatacctgtccgcctttctcccttcgggaagcgtggcgctttctcatagctcacgctgtaggtatctc
agttcggtgtaggtcgttcgctccaagctgggctgtgtgcacgaaccccccgttcagcccgaccgctgcgccttatccggtaactatcgtctt
gagtccaacccggtaagacacgacttatcgccactggcagcagccactggtaacaggattagcagagcgaggtatgtaggcggtgctaca
gagttcttgaagtggtggcctaactacggctacactagaaggacagtatttggtatctgcgctctgctgaagccagttaccttcggaaaaaga
gttggtagctcttgatccggcaaacaaaccaccgctggtagcggtggtttttttgtttgcaagcagcagattacgcgcagaaaaaaaggatct
caagaagatcctttgatcttttctacggggtctgacgctcagtggaacgaaaactcacgttaagggattttggtcatgagattatcaaaaaggat
cttcacctagatccttttaaattaaaaatgaagttttaaatcaatctaaagtatatatgagtaaacttggtctgacagttaccaatgcttaatcagtg
aggcacctatctcagcgatctgtctatttcgttcatccatagttgcctgactccccgtcgtgtagataactacgatacgggagggcttaccatct
ggccccagtgctgcaatgataccgcgagacccacgctcaccggctccagatttatcagcaataaaccagccagccggaagggccgagc
gcagaagtggtcctgcaactttatccgcctccatccagtctattaattgttgccgggaagctagagtaagtagttcgccagttaatagtttgcgc
aacgttgttgccattgctacaggcatcgtggtgtcacgctcgtcgtttggtatggcttcattcagctccggttcccaacgatcaaggcgagttac
atgatcccccatgttgtgcaaaaaagcggttagctccttcggtcctccgatcgttgtcagaagtaagttggccgcagtgttatcactcatggtta
tggcagcactgcataattctcttactgtcatgccatccgtaagatgcttttctgtgactggtgagtactcaaccaagtcattctgagaatagtgtat
gcggcgaccgagttgctcttgcccggcgtcaatacgggataataccgcgccacatagcagaactttaaaagtgctcatcattggaaaacgtt
cttcggggcgaaaactctcaaggatcttaccgctgttgagatccagttcgatgtaacccactcgtgcacccaactgatcttcagcatcttttactt
tcaccagcgtttctgggtgagcaaaaacaggaaggcaaaatgccgcaaaaaagggaataagggcgacacggaaatgttgaatactcatac
tcttcctttttcaatattattgaagcatttatcagggttattgtctcatgagcggatacatatttgaatgtatttagaaaaataaacaaataggggttc
cgcgcacatttccccgaaaagtgccac
SEQ ID NO: 7 (vHVT514 nucleotide sequence) (vHVT514)
accgccagatatccaaagtacagcacgatgttgcacggacgactttgcagtcaccagccttcctttccacccccccaccaacaaaatgtttat
cgtaggacccatatccgtaataaggatgggtctggcagcaaccccataggcgcctcggcgtggtagttctcgaggatacatccaaagaggt
tgagtattctctctacacttcttgttaaatggaaagtgcatttgcttgttcttacaatcggcccgagtctcgttcacagcgcctcgttcacacttaaa
ccacaaatagtctacaggctatatgggagccagactgaaactcacatatgactaatattcgggggtgttagtcacgtgtagcccattgtgtgc
atataacgatgttggacgcgtccttattcgcggtgtacttgatactatggcagcgagcatgggatattcatcctcgtcatcgttaacatctctacg
ggttcagaatgtttggcatgtcgtcgatcctttgcccatcgttgcaaattacaagtccgatcgccatgaccgcgataagcctgtaccatgtggc
attagggtgacatctcgatcatacattataagaccaacgtgcgagtcttccaaagacctgcacgccttcttcttcggattgtcaacgggttcttc
agaatctatgcccatatctggcgttgagaccattgtgcgtttaatgaacaataaagcggcatgccatggaaaggagggctgcagatctccatt
ttctcacgccactatcctggacgctgtagacgataattataccatgaatatagagggggtatgtttccactgccactgtgatgataagttttctcc
agattgttggatatctgcattttctgctgccgaacaaacttcatcgctatgcaaagagatgcgtgtgtacacgcgccgttgagtatacgggaaa
ctaaatgttcatagaggtctttgggctatatgttattaaataaaataattgtcgactctagaggatccgaaaaaacctcccacacctccccctgaa
cctgaaacataaaatgaatgcaattgttgttgttaacttgtttattgcagcttataatggttacaaataaagcaatagcatcacaaatttcacaaata
aagcatttttttcactgcattctagttgtggtttgtccaaactcatcaatgtatcttatcatgtctggatccccgcggtcattagatgcagatgttgca
acggcaagaaccgttagacatagcccagaacaggaaagcagccagacccatagccagaaccagagaagaagcaacggtagagtagat
ggtcaggattttgtaggtaccttcagattccagtttaacaccttcgattttctgacgttccagacgagattcttctttgtatttcccacggttgtaggt
accgttacggatggtttccatgcactggtcgtcgcatttgtggtacagttcgaagcaacctttaccgtcttccatagcgttagaacccagagca
cgtttaactttgttgtacaggttgttaacgttagcgtcgtgttcgtccagggttttctggttttccagcagaaccagcagttcagcgttgtaagccc
aaacgtcctggatctggtcgtcgattttgttgttgatcatgttcagacgggtttcaacttcagagaattcgtggtcgatgatttcgtactgtttgttc
attttgtcaacgatgttgttaactttagaggtgattttgtcaacagctttctgggtagaatcacggtcagcagccataccaacaccctggtcgtta
gagtgctggaaaccgtaccaaccagcaaccagacccggccaaccaccttcgatgaaaccagcgatagcaccgaacagaccacgggaa
gacctagccggaacgttacgcagaccgatagccagtttcagagatttaacaccgatgtatttcgggcaagtaccgaaagcgtatttagagat
gttgtggaacggcagggtagagttcagaccacctttttcggtctggcactgaacaacgcagttaccagagttcagatcggttttcaggatacg
accgtgagattcaccagacagaacgtgaccgaaccacggagcgatcaggttaccgttagaacgaacacgcagggtctgacccggtttca
gaacagaccagtagtagttgatacgaccgatcagaccgttaaccagcggacgcggaccgatcagcggtttgaaggtacggtccaggttttc
ggtggtaacagaggtggtggtgtcggtacgggtgtacaggttggtctgagcggtgtcggtcggcgggtggtggataccccaaacgaaca
ggatgtctttaccacggttgttggtgaactgagcgtcctgaaccgggtacagaccgtttttctgggtcagccaacgcatgttacggtagaaag
agtcagagcaagatttagaggtaccggtgtaggtaacgttccagatggtgtccgggaagatctggatacgctggtaagaagaagaagaag
agaacagggtacgcagttcttccaggttttcaacgttacccgggtagcaggtaccgttaacagcagacggacgttcaacgatgtaagaccat
tcacgaccacccagcagcaggtcgcaagacgggttaccgtagatcagaccttcaatagtgcaggtgttcaggatcagcgggtgacccagg
ttggtagcgcacagcataccgttgtgttcggtgtgcagcagttctttagcgtgggtaaccggaacgttggtttcggtcagggtgtcaacggttt
cggtagagttggtagactggtgaccgatgcagattttgtcagcgttagaggtggtaaccagcagcaggataatcatcaggctaattggttcca
tggtattatcatcgtgtttttcaaaggaaaaccacgtccccgtggttcggggggcctagacgttttttaacctcgactaaacacatgtaaagcat
gtgcaccgaggccccagatcagatcccatacaatggggtaccttctgggcatccttcagccccttgttgaatacgcttgaggagagccatttg
actctttccacaactatccaactcacaacgtggcactggggttgtgccgcctttgcaggtgtatcttatacacgtggcttttggccgcagaggc
acctgtcgccaggtggggggttccgctgcctgcaaagggtcgctacagacgttgtttgtcttcaagaagcttccagaggaactgcttccttca
cgacattcaacagaccttgcattcctttggcgagaggggaaagacccctaggaatgctcgtcaagaagacagggccaggtttccgggccc
tcacattgccaaaagacggcaatatggtggaaaataacatatagacaaacgcacaccggccttattccaagcggcttcggccagtaacgtta
gggggggggggggcggccgctcatgctcttgtagtggctctcatctgatcgagggtattattcccaagccatagcaaggtcttttgttgtgcc
ttctgtttgtacatcaggtaacacgctaacaccagactaagtgcaccgaaaactagagaaatgacagttagaacaatataggtaatgagagc
agatgtgctggttagtctgacattgactttttctagcttgctgttgctttctgccaacctatccaaggcattgctgattgaattgttgacgtttccaag
ttcagttgatatatcaagattgcctgtcacgatgacttgagaatctagtattgagatgttcttttgataagttgcatcaaattccccactgagcctta
gagttatcccgtctaatgataagacattgcacgaatgtctatctatcagggatacagcttctccataattttgcgatatgataccaggagggtctg
tacatctacatgttgttattttacaattggcaataactgagcctttaagggccatatacggcgtagtgagtgcgccttcagtctttgaatacatgca
agctgatgtgttgccgctcaaacaggaataaatacctggggacatggggaatgtcactattctagtacaatataaatccagatcggactctata
cagtatgaggtgtcaagctcttctatcacggaaccgacttgtgtcactactttcgggacaagtgctgaggcatatcctttggttgtacttacagat
aaggtctccaaataggtggcacgcatattatttaagttcccgactgagggtaaattcacttgtatgcccaagagttgagtctgtgagtcatacag
tatagggtaaccagtgatcaggccgctaccaattaacgagctgagttgattgttccctatacctaacttagttaataagtaatccatattgccacc
agctaaattataaagtgcctggatggtcagctgagttaatgcaggggaggtgatctgtggcccgaatactgtagtcaattcagttaggtatag
gttgagttctacaccaacctgttgtgtgatttttatacagtccaattctcgcgccgtattattaaactggtcattgacaaactgctgcatcttcccaa
ctgccactgatagttgtgataatccgtcggtgacttcatgcacagcttcattggttgcagcaatgctctccttaagccggaggatgttggcggc
attctggttggcttgtattagggccgcagctgctgttatctgtgccgctgttgcaaccccaagagctacactgccaataacagcacctatcagg
cggccttgcttgcctcctccagatgtggacacagacccttggatcttgcggatggagtcgccaagaggagtgagcaaagtagtcagtgttct
gttatatgcctctaatggggcttttgcacacgcctccttatccctgggcatattcgggagcaacttgactatgattgaccctgtctgagacgaag
tgtatacattgactgccttatctcctgttactacaattcctgcagctgcaagaggcctgccgtcaagagagcttgtcggacggatacagcccaa
tatcagcataatccgggtgatcagcatcagaggtgctgggatcctggtagaaggtttggagcccatgcggccgcctgcagcgaggagctct
gcgttctacggtggtcagaccgaagactgcgacggtaccgacgctggtcgcgcctcttatacccacgtagaacgcagctcagccaataga
atgcgtgccaatatggaatttccaggggaaaaccggggcggtgttacgttttggctgccctttcacttcccattgacgtgtattggctcgagaa
cggtactttcccattaatcagctatgggaaagtaccgtttaaaggtcacgttgcattagtttcaatagcccattgacgtcaatggtgggaaagta
catggcgttttaattaaattggctggaaaaacccaatgactcacccctattgaccttatgtacgtgccaataatgggaaaaacccattgactcac
cccctattgaccttttgtactgggcaaaacccaatggaaagtccctattgactcagtgtacttggctccaatgggactttcctgttgattcacccc
tattgaccttatgtactgggcaaaacccattggaaagtccctaatgactcagtatacgtgccagtaatgggaaaaacccattggcttacctccc
attgaccttatgtactgggcaaaacccattggaaagtccctattgactcaatgtacttggctccaatgggactttcctgttgactcaccccctatt
gaccttatgtactgggcaaaacccaatggaaagtccctattgagtcagtgtacttggctccaatgggtttttcccattgactcatcccctattgac
cttatgtactgggcaaaacccaatggaaagtccctattgacgcagtgtacttggctccaatgggactttcctgttgattcaccccctattgacctt
atgtactgggcagaatacaatggaaagtccctattgactcacccacattgaccttatatgcttgccaacaatggaaaaacccattggaaagtc
cctattgagtcagtgtacttggcagcaatgggtttttcccattggctcacctcccattgacccaatgtacttgggcaaaacccagtggaaagtc
ccatttgactcagtgtgcttgccagtaatgggaaaaacccattggcttacctcccattgacccaatgtacttgggtaaaggccattgagtcacc
acccctatgctgggaaatggtgaacgccccctatgtggaaagtccctatgggccgccattagagtgcatgaccgtgctgattcatatgccata
tgagtgtattagggggctttccgcttgggaaattgggtaaaaagtccccgtattactcacatagggggcgtttggctttgcaaattaggggattt
cagtgcatttggcattaaaaactattggttctagtcataaaacgggcggagttgggggatccactagtgaattcgtttaatgttagtttattcaatg
cattggttgcaaatattcattacttctccaatcccaggtcattctttagcgagatgatgttatgacattgctgtgaaaattactacaggatatattttta
agatgcaggagtaacaatgtgcatagtaggcgtagttatcgcagacgtgcaacgcttcgcatttgagttaccgaagtgcccaacagtgctgc
ggttatggtttatgcgcacagaatccatgcatgtcctaattgaaccatccgatttttcttttaatcgcgatcgttgtttgggcaactgcgttatttcag
atctaaaaaatttaccctttatgaccatcacatctctctggctcataccccgcttggataagatatcatgtagattccgccctaagaaatgcaaac
taacattattgtcggttccatatacacttccatcttgtccttcgaaaataacaaactcgcgcaatagaccgtccgtacatgcatggccgatgtgtg
tcaacatcattggtctgctagatcccgatgggacgaatcgtacagtcgtcgctccagcattggcaaaaatccccagataccctccatgcggc
aaatctaaattgcgaccccgaagagactgcaccaaagtcttatcgacgcacgctgatttttttgaacagcgggagcccattatcttcagtgga
gcgtagacgggcgaggctaattatgtgacatagcaacactgcatgtatgtttttataaatcaataagagtacataatttattacgtatcatttccgt
ttgtaatatactgtatacatcatccacactattagtcagcactagcgcgcgggcgcacgttacaatagcagcgtgcccgttatctatattgtccg
atatttacacataacatttcatcgacatgattaaatacctaagtactgcacacagatgtttaatgtatatcgtcatataaattatatcgctaggacag
acccaaacgacctttatcccaaacagtcagatcctcttctcaagtgtcgatttctgttatggaatatgcataccctggcccagaaattgcacgca
cgagcgtagtgaatgcgtcattggttttacatttaaaggctaaatgcacaaattctttagacgacagcacatcgttaaatagcatctctagcgttc
ttatgaatgctaagcattggagtcctcctggtcggccacaataacagctgagtatcataccctgagctccggggttgtcgcacatagcggatt
cgtataaacataggattttccgcgaatccatcagttgcaaaaatctgttaggctccatcaacaacgctggatttacttcagatccacgcgtaaag
taatggtgctcgaataccgtttttagagttgtcggcatttcaaggaacaaaga
SEQ ID NO: 8 (mCMV IE promoter nucleotide sequence)
aactccgcccgttttatgactagaaccaatagtttttaatgccaaatgcactgaaatcccctaatttgcaaagccaaacgccccctatgtgagta
atacggggactttttacccaatttcccaagcggaaagccccctaatacactcatatggcatatgaatcagcacggtcatgcactctaatggcg
gcccatagggactttccacatagggggcgttcaccatttcccagcataggggtggtgactcaatggcctttacccaagtacattgggtcaatg
ggaggtaagccaatgggtttttcccattactggcaagcacactgagtcaaatgggactttccactgggttttgcccaagtacattgggtcaatg
ggaggtgagccaatgggaaaaacccattgctgccaagtacactgactcaatagggactttccaatgggtttttccattgttggcaagcatata
aggtcaatgtgggtgagtcaatagggactttccattgtattctgcccagtacataaggtcaatagggggtgaatcaacaggaaagtcccattg
gagccaagtacactgcgtcaatagggactttccattgggttttgcccagtacataaggtcaataggggatgagtcaatgggaaaaacccatt
ggagccaagtacactgactcaatagggactttccattgggttttgcccagtacataaggtcaatagggggtgagtcaacaggaaagtcccat
tggagccaagtacattgagtcaatagggactttccaatgggttttgcccagtacataaggtcaatgggaggtaagccaatgggtttttcccatt
actggcacgtatactgagtcattagggactttccaatgggttttgcccagtacataaggtcaataggggtgaatcaacaggaaagtcccattg
gagccaagtacactgagtcaatagggactttccattgggttttgcccagtacaaaaggtcaatagggggtgagtcaatgggtttttcccattatt
ggcacgtacataaggtcaataggggtgagtcattgggtttttccagccaatttaattaaaacgccatgtactttcccaccattgacgtcaatggg
ctattgaaactaatgcaacgtgacctttaaacggtactttcccatagctgattaatgggaaagtaccgttctcgagccaatacacgtcaatggg
aagtgaaagggcagccaaaacgtaacaccgccccggttttcccctggaaattccatattggcacgcattctattggctgagctgcgttctacg
tgggtataagaggcgcgaccagcgtcggtaccgtcgcagtcttcggtctgaccaccgtagaacgcagagctcctcgctgcag
SEQ ID NO: 9 (codon-optimised AIV H9-HA nucleotide sequence)
atggaaccaattagcctgatgattatcctgctgctggttaccacctctaacgctgacaaaatctgcatcggtcaccagtctaccaactctaccg
aaaccgttgacaccctgaccgaaaccaacgttccggttacccacgctaaagaactgctgcacaccgaacacaacggtatgctgtgcgctac
caacctgggtcacccgctgatcctgaacacctgcactattgaaggtctgatctacggtaacccgtcttgcgacctgctgctgggtggtcgtga
atggtcttacatcgttgaacgtccgtctgctgttaacggtacctgctacccgggtaacgttgaaaacctggaagaactgcgtaccctgttctctt
cttcttcttcttaccagcgtatccagatcttcccggacaccatctggaacgttacctacaccggtacctctaaatcttgctctgactctttctaccgt
aacatgcgttggctgacccagaaaaacggtctgtacccggttcaggacgctcagttcaccaacaaccgtggtaaagacatcctgttcgtttg
gggtatccaccacccgccgaccgacaccgctcagaccaacctgtacacccgtaccgacaccaccacctctgttaccaccgaaaacctgga
ccgtaccttcaaaccgctgatcggtccgcgtccgctggttaacggtctgatcggtcgtatcaactactactggtctgttctgaaaccgggtcag
accctgcgtgttcgttctaacggtaacctgatcgctccgtggttcggtcacgttctgtctggtgaatctcacggtcgtatcctgaaaaccgatct
gaactctggtaactgcgttgttcagtgccagaccgaaaaaggtggtctgaactctaccctgccgttccacaacatctctaaatacgctttcggt
acttgcccgaaatacatcggtgttaaatctctgaaactggctatcggtctgcgtaacgttccggctaggtcttcccgtggtctgttcggtgctatc
gctggtttcatcgaaggtggttggccgggtctggttgctggttggtacggtttccagcactctaacgaccagggtgttggtatggctgctgacc
gtgattctacccagaaagctgttgacaaaatcacctctaaagttaacaacatcgttgacaaaatgaacaaacagtacgaaatcatcgaccacg
aattctctgaagttgaaacccgtctgaacatgatcaacaacaaaatcgacgaccagatccaggacgtttgggcttacaacgctgaactgctg
gttctgctggaaaaccagaaaaccctggacgaacacgacgctaacgttaacaacctgtacaacaaagttaaacgtgctctgggttctaacgc
tatggaagacggtaaaggttgcttcgaactgtaccacaaatgcgacgaccagtgcatggaaaccatccgtaacggtacctacaaccgtggg
aaatacaaagaagaatctcgtctggaacgtcagaaaatcgaaggtgttaaactggaatctgaaggtacctacaaaatcctgaccatctactct
accgttgcttcttctctggttctggctatgggtctggctgctttcctgttctgggctatgtctaacggttcttgccgttgcaacatctgcatctaatga
SEQ ID NO: 10 (IBDV VP2 nucleotide sequence)
atgacaaacctgcaagatcaaacccaacagattgttccgttcatacggagccttctgatgccaacaaccggaccggcgtccattccggacg
acaccctggagaagcacactctcaggtcagagacctcgacctacaatttgactgtgggggacacagggtcagggctaattgtctttttccctg
gattccctggctcaattgtgggtgctcactacacactgcagagcaatgggaactacaagttcgatcagatgctcctgactgcccagaacctac
cggccagctacaactactgcagactagtgagtcggagtctcacagtgaggtcaagcacactccctggtggcgtttatgcactaaacggcac
cataaacgccgtgaccttccaaggaagcctgagtgaactgacagatgttagctacaatgggttgatgtctgcaacagccaacatcaacgac
aaaattgggaatgtcctggtaggggaaggggtcactgtcctcagcctacccacatcatatgatcttgggtatgtgaggcttggtgaccccatt
cccgctatagggcttgacccaaaaatggtagctacatgcgacagcagtgacaggcccagagtctacaccataactgcagccgatgattacc
aattctcatcacagtaccaaccaggtggggtaacaatcacactgttctcagccaacattgatgctatcacaagcctcagcattgggggagag
ctcgtgtttcaaacaagcgtccaaggccttgtactgggcgccaccatctaccttataggctttgatgggactgcggtaatcaccagagctgta
gccgcagataatgggctgacggccggcaccgacaatcttatgccattcaatcttgtcattccaaccaatgagataacccagccaatcacatc
catcaaactggagatagtgacctccaaaagtggtggtcaggcaggggatcagatgtcatggtcggcaagtgggagcctagcagtgacgat
ccatggtggcaactatccaggggccctccgtcccgtcacactagtagcctacgaaagagtggcaacaggatccgtcgttacggtcgctgg
ggtgagtaacttcgagctgattccaaatcctgaactagcaaagaacctggttacagaatacggccgatttgacccaggagccatgaactaca
caaaattgatactgagtgagagggaccgtcttggcatcaagaccgtctggccaacaagggagtacactgattttcgtgagtacttcatggag
gtggccgacctcaactctcccctgaagattgcaggagcatttggcttcaaagacataatccgggctataaggaggtaa
SEQ ID NO: 11 (NDV F nucleotide sequence)
atgggctccaaaccttctaccaggatcccagcacctctgatgctgatcacccggattatgctgatattgggctgtatccgtccgacaagctctc
ttgacggcaggcctcttgcagctgcaggaattgtagtaacaggagataaggcagtcaatgtatacacttcgtctcagacagggtcaatcata
gtcaagttgctcccgaatatgcccagggataaggaggcgtgtgcaaaagccccattagaggcatataacagaacactgactactttgctcac
tcctcttggcgactccatccgcaagatccaagggtctgtgtccacatctggaggaggcaagcaaggccgcctgataggtgctgttattggca
gtgtagctcttggggttgcaacagcggcacagataacagcagctgcggccctaatacaagccaaccagaatgccgccaacatcctccggc
ttaaggagagcattgctgcaaccaatgaagctgtgcatgaagtcaccgacggattatcacaactatcagtggcagttgggaagatgcagca
gtttgtcaatgaccagtttaataatacggcgcgagaattggactgtataaaaatcacacaacaggttggtgtagaactcaacctatacctaact
gaattgactacagtattcgggccacagatcacctcccctgcattaactcagctgaccatccaggcactttataatttagctggtggcaatatgg
attacttattaactaagttaggtatagggaacaatcaactcagctcgttaattggtagcggcctgatcactggttaccctatactgtatgactcac
agactcaactcttgggcatacaagtgaatttaccctcagtcgggaacttaaataatatgcgtgccacctatttggagaccttatctgtaagtaca
accaaaggatatgcctcagcacttgtcccgaaagtagtgacacaagtcggttccgtgatagaagagcttgacacctcatactgtatagagtcc
gatctggatttatattgtactagaatagtgacattccccatgtccccaggtatttattcctgtttgagcggcaacacatcagcttgcatgtattcaa
agactgaaggcgcactcactacgccgtatatggcccttaaaggctcagttattgccaattgtaaaataacaacatgtagatgtacagaccctc
ctggtatcatatcgcaaaattatggagaagctgtatccctgatagatagacattcgtgcaatgtcttatcattagacgggataactctaaggctc
agtggggaatttgatgcaacttatcaaaagaacatctcaatactagattctcaagtcatcgtgacaggcaatcttgatatatcaactgaacttgg
aaacgtcaacaattcaatcagcaatgccttggataggttggcagaaagcaacagcaagctagaaaaagtcaatgtcagactaaccagcaca
tctgctctcattacctatattgttctaactgtcatttctctagttttcggtgcacttagtctggtgttagcgtgttacctgatgtacaaacagaaggca
caacaaaagaccttgctatggcttgggaataataccctcgatcagatgagagccactacaagagcatg
SEQ ID NO: 12 (IRES nucleotide sequence)
cccccccccctaacgttactggccgaagccgcttggaataaggccggtgtgcgtttgtctatatgttattttccaccatattgccgtcttttggca
atgtgagggcccggaaacctggccctgtcttcttgacgagcattcctaggggtctttcccctctcgccaaaggaatgcaaggtctgttgaatg
tcgtgaaggaagcagttcctctggaagcttcttgaagacaaacaacgtctgtagcgaccctttgcaggcagcggaaccccccacctggcga
caggtgcctctgcggccaaaagccacgtgtataagatacacctgcaaaggcggcacaaccccagtgccacgttgtgagttggatagttgtg
gaaagagtcaaatggctctcctcaagcgtattcaacaaggggctgaaggatgcccagaaggtaccccattgtatgggatctgatctggggc
ctcggtgcacatgctttacatgtgtttagtcgaggttaaaaaacgtctaggccccccgaaccacggggacgtggttttcctttgaaaaacacg
atgataat
SEQ ID NO: 13 (SV40 polyA nucleotide sequence)
ggggatccagacatgataagatacattgatgagtttggacaaaccacaactagaatgcagtgaaaaaaatgctttatttgtgaaatttgtgatg
ctattgctttatttgtaaccattataagctgcaataaacaagttaacaacaacaattgcattcattttatgtttcaggttcagggggaggtgtggga
ggtttttt
SEQ ID NO: 14 (P2A of pFP2AVP2)
ggctccggcgccaccaacttctccctgctcaagcaggccggcgacgtggaggagaaccctggacct
SEQ ID NO: 15 (MB080 primer)
CGAACAAACTTCATCGCTATG C
SEQ ID NO: 16 (MB081 primer)
TAACTCAAATGCGAAGCGTTGC
SEQ ID NO: 17 (SAH9F.3 primer)
CTCTGAAGTTGAAACCCGTC
SEQ ID NO: 18 (VP2.F primer)
AGA TTG CAG GAG CAT TTG GCT
SEQ ID NO: 19 (SV40polyA.R1 primer)
ATGCAATTGTTGTTAACT
SEQ ID NO: 20 (mCMVF primer)
AACTCCGCCCGTTTTATG
SEQ ID NO: 21 (110P.1 primer)
TCT TTG TTC CTT GAA ATG CCG ACA
SEQ ID NO: 22 (110P1 primer)
TCA TGT CGA TGA AAT GTT ATG
SEQ ID NO: 23 (110P2 primer)
TGC GTC GAT AAG ACT TTG GT
SEQ ID NO: 24 (119P1 primer)
TTA GTT TGC ATT TCT TAG GG
SEQ ID NO: 25 (119P2 primer)
TGA ATA TTT GCA ACC AAT GC
SEQ ID NO: 26 (mCMVP1 primer)
TCA GCA CGG TCA TGC ACT CT
SEQ ID NO: 27 (309P1 primer)
ATG TAC TTT CCC ACC ATT GAC G
SEQ ID NO: 28 (309P2 primer)
AGG CGG CCG CTC TAG AAC G
SEQ ID NO: 29 (309P3 primer)
ACC GGC CAG CTA CAA CTA
SEQ ID NO: 30 (309P4 primer)
ATG CGA CAG CAG TGA CAG
SEQ ID NO: 31 (309P5 primer)
AGA TAA CCC AGC CAA TCA
SEQ ID NO: 32 (309P6 primer)
ACT GAG TGA GAG GGA CCG TC
SEQ ID NO: 33 (SAH9R primer)
ATC AGG CTA ATT GGT TCC AT
SEQ ID NO: 34 (SAH9F primer)
ATG GAA CCA ATT AGC CTG AT
SEQ ID NO: 35 (SAH9F.1 primer)
ATG GAA CCA ATT AGC CTG AT
SEQ ID NO: 36 (SAH9F.2 primer)
ACG TTC TGT CTG GTG AAT CT
SEQ ID NO: 37 (309P15R primer)
ACA CGC GCC GTT GAG TAT
SEQ ID NO: 38 (309P15 primer)
ATA CTC AAC GGC GCG TGT
SEQ ID NO: 39 (309P16 primer)
ATC CGA AGA AGA AGG CGT
SEQ ID NO: 40 (309P18 primer)
ACC GCC AGA TAT CCA AAG TA
SEQ ID NO: 41 (312P6 primer)
CGT CAA CAA TTC AAT CAG CA
SEQ ID NO: 42 (312P2 primer)
TAT GCC CAG GGA TAA GGA GG
SEQ ID NO: 43 (312P3R primer)
ACT TCA TGC ACA GCT TCA
SEQ ID NO: 44 (312P4 primer)
CTG GTG GCA ATA TGG ATT
SEQ ID NO: 45 (312P5R primer)
ATG TGT TGC CGC TCA AAC AG

1. A recombinant herpesvirus of turkeys (HVT) vector comprising a first heterologous polynucleotide encoding a first avian antigen and a second heterologous polynucleotide encoding a second avian antigen, wherein the first and second heterologous polynucleotides are inserted in the same non-essential site in the recombinant HVT vector genome, and wherein the first or second antigen is an avian influenza virus (AIV) subtype H9 haemagglutinin (AIV H9-HA) antigen.

2. The recombinant HVT vector of claim 1, wherein the AIV H9-HA antigen is the second antigen.

3. The recombinant HVT vector of claim 1, wherein the first heterologous polynucleotide is operably linked to a murine cytomegalovirus immediate early (mCMV IE) promoter.

4. The recombinant HVT vector of claim 1, wherein the first heterologous polynucleotide is operably linked to a promoter comprising a nucleotide sequence having at least 80% identity to SEQ ID NO: 8 or a fragment thereof and having equivalent activity thereto.

5. The recombinant HVT vector of claim 1, wherein the polynucleotide encoding the AIV H9-HA antigen comprises a polynucleotide sequence having at least 80% identity to SEQ ID NO: 9, and/or the AIV H9-HA antigen comprises an amino acid sequence having at least 80% identity to SEQ ID NO: 1.

6. The recombinant HVT vector of claim 1, wherein the polynucleotide encoding the AIV H9-HA antigen is codon-optimised for expression in an avian host.

7. The recombinant HVT vector of claim 1, wherein the first heterologous polynucleotide is linked via an internal ribosome entry site (IRES) sequence or a sequence encoding a self-cleaving porcine teschovirus-1 2A or foot and mouth disease virus peptide (P2A), to the second heterologous polynucleotide.

8. The recombinant HVT vector of claim 1, wherein the first avian antigen is different from the second avian antigen.

9. The recombinant HVT vector of claim 1, wherein the first avian antigen is an infectious bursal disease virus VP2 (IBDV VP2) antigen or a Newcastle disease virus F protein (NDV-F) antigen.

10. The recombinant HVT vector of claim 9, wherein the IBDV VP2 antigen comprises an amino acid sequence having at least 80% identity to SEQ ID NO: 2.

11. The recombinant HVT vector of claim 9, wherein the NDV-F antigen comprises an amino acid sequence having at least 80% identity to SEQ ID NO: 5.

12. The recombinant HVT vector of claim 1, wherein the recombinant HVT vector further comprises an SV40 polyadenylation signal.

13. The recombinant HVT vector of claim 1, wherein the recombinant HVT vector comprises an expression cassette with the following elements:
(a) a mCMV IE promoter;
(b) the first heterologous polynucleotide;
(c) the second heterologous polynucleotide; and
(d) an SV40 polyadenylation signal.

14. The recombinant HVT vector of claim 1, wherein the recombinant HVT vector comprises an expression cassette with the following elements in the following order:
(a) a mCMV IE promoter;
(b) the first heterologous polynucleotide;
(c) the second heterologous polynucleotide; and
(d) an SV40 polyadenylation signal.

15. The recombinant HVT vector of claim 1, wherein the recombinant HVT vector comprises an expression cassette which further comprises an IRES or P2A sequence, between the first and the second heterologous polynucleotide.

16. The recombinant HVT vector of claim 1, wherein the non-essential site in the recombinant HVT vector genome is an IG1 site.

17. The recombinant HVT vector of claim 1, wherein the recombinant HVT vector is capable of inducing a protective immune response against AIV and the avian pathogen from which the first avian antigen is derived.

18. A nucleic acid molecule, a cell, or a composition comprising the recombinant HVT vector of claim 1.

19. An antigen expression cassette comprising the following elements:
(a) a mCMV IE promoter;
(b) a first heterologous polynucleotide encoding a first avian antigen;
(c) an IRES or P2A sequence;
(d) a second heterologous polynucleotide encoding a second avian antigen; and
(e) an SV40 polyadenylation signal,
wherein the first or second antigen is an AIV antigen.

20-23. (canceled)

24. A method of inducing a protective immune response against AIV, the avian pathogen from which the additional avian antigen is derived and/or MDV in an avian, comprising administering the recombinant HVT vector of claim 1 to the avian.

25. (canceled)

26. The method of claim 24, wherein inducing a protective immune response comprises reducing clinical signs associated with infection by AIV, the avian pathogen from which the additional avian antigen is derived and/or MDV in an avian, and the clinical signs are respiratory clinical signs.

27-28. (canceled)

29. A method of reducing AIV shedding in an avian, comprising administering the recombinant HVT vector of claim 1 to the avian.


SEQUENCE LISTING (45 sequences)

SEQ ID NO: 1 - protein, 560 aa, Alphainfluenzavirus influenzae
MEPISLMIILLLVTTSNADKICIGHQSTNSTETVDTLTETNVPVTHAKELLHTEHNGMLCATNLGHPLILNTCTIEGLIYGNPSCDLLLGGREWSYIVERPSAVNGTCYPGNVENLEELRTLFSSSSSYQRIQIFPDTIWNVTYTGTSKSCSDSFYRNMRWLTQKNGLYPVQDAQFTNNRGKDILFVWGIHHPPTDTAQTNLYTRTDTTTSVTTENLDRTFKPLIGPRPLVNGLIGRINYYWSVLKPGQTLRVRSNGNLIAPWFGHVLSGESHGRILKTDLNSGNCVVQCQTEKGGLNSTLPFHNISKYAFGTCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRGKYKEESRLERQKIEGVKLESEGTYKILTIYSTVASSLVLAMGLAAFLFWAMSNGSCRCNICI

SEQ ID NO: 2 - protein, 453 aa, Infectious bursal disease virus
MTNLQDQTQQIVPFIRSLLMPTTGPASIPDDTLEKHTLRSETSTYNLTVGDTGSGLIVFFPGFPGSIVGAHYTLQSNGNYKFDQMLLTAQNLPASYNYCRLVSRSLTVRSSTLPGGVYALNGTINAVTFQGSLSELTDVSYNGLMSATANINDKIGNVLVGEGVTVLSLPTSYDLGYVRLGDPIPAIGLDPKMVATCDSSDRPRVYTITAADDYQFSSQYQPGGVTITLFSANIDAITSLSIGGELVFQTSVQGLVLGATIYLIGFDGTAVITRAVAADNGLTAGTDNLMPFNLVIPTNEITQPITSIKLEIVTSKSGGQAGDQMSWSASGSLAVTIHGGNYPGALRPVTLVAYERVATGSVVTVAGVSNFELIPNPELAKNLVTEYGRFDPGAMNYTKLILSERDRLGIKTVWPTREYTDFREYFMEVADLNSPLKIAGAFGFKDIIRAIRR

SEQ ID NO: 3 - DNA, 7278 bp, synthetic construct
  source 1..7278: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccgcggccgctctagaactcgtcgatcgcagcgatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaaattgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtagccgcagataatgggctgacggccggcaccgacaatcttatgccattcaatcttgtcattccaaccaatgagataacccagccaatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgattccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggaaccaattagcctgatgattatcctgctgctggttaccacctctaacgctgacaaaatctgcatcggtcaccagtctaccaactctaccgaaaccgttgacaccctgaccgaaaccaacgttccggttacccacgctaaagaactgctgcacaccgaacacaacggtatgctgtgcgctaccaacctgggtcacccgctgatcctgaacacctgcactattgaaggtctgatctacggtaacccgtcttgcgacctgctgctgggtggtcgtgaatggtcttacatcgttgaacgtccgtctgctgttaacggtacctgctacccgggtaacgttgaaaacctggaagaactgcgtaccctgttctcttcttcttcttcttaccagcgtatccagatcttcccggacaccatctggaacgttacctacaccggtacctctaaatcttgctctgactctttctaccgtaacatgcgttggctgacccagaaaaacggtctgtacccggttcaggacgctcagttcaccaacaaccgtggtaaagacatcctgttcgtttggggtatccaccacccgccgaccgacaccgctcagaccaacctgtacacccgtaccgacaccaccacctctgttaccaccgaaaacctggaccgtaccttcaaaccgctgatcggtccgcgtccgctggttaacggtctgatcggtcgtatcaactactactggtctgttctgaaaccgggtcagaccctgcgtgttcgttctaacggtaacctgatcgctccgtggttcggtcacgttctgtctggtgaatctcacggtcgtatcctgaaaaccgatctgaactctggtaactgcgttgttcagtgccagaccgaaaaaggtggtctgaactctaccctgccgttccacaacatctctaaatacgctttcggtacttgcccgaaatacatcggtgttaaatctctgaaactggctatcggtctgcgtaacgttccggctaggtcttcccgtggtctgttcggtgctatcgctggtttcatcgaaggtggttggccgggtctggttgctggttggtacggtttccagcactctaacgaccagggtgttggtatggctgctgaccgtgattctacccagaaagctgttgacaaaatcacctctaaagttaacaacatcgttgacaaaatgaacaaacagtacgaaatcatcgaccacgaattctctgaagttgaaacccgtctgaacatgatcaacaacaaaatcgacgaccagatccaggacgtttgggcttacaacgctgaactgctggttctgctggaaaaccagaaaaccctggacgaacacgacgctaacgttaacaacctgtacaacaaagttaaacgtgctctgggttctaacgctatggaagacggtaaaggttgcttcgaactgtaccacaaatgcgacgaccagtgcatggaaaccatccgtaacggtacctacaaccgtgggaaatacaaagaagaatctcgtctggaacgtcagaaaatcgaaggtgttaaactggaatctgaaggtacctacaaaatcctgaccatctactctaccgttgcttcttctctggttctggctatgggtctggctgctttcctgttctgggctatgtctaacggttcttgccgttgcaacatctgcatctaatgaccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagacctgcagggtcgacaattattttatttaataacatatagcccaaagacctctatgaacatttagtttcccgtatactcaacggcgcgtgtacacacgcatctctttgcatagcgatgaagtttgttcggcagcagaaaatgcagatatccaacaatctggagaaaacttatcatcacagtggcagtggaaacataccccctctatattcatggtataattatcgtctacagcgtccaggatagtggcgtgagaaaatggagatctgcagccctcctttccatggcatgccgctttattgttcattaaacgcacaatggtctcaacgccagatatgggcatagattctgaagaacccgttgacaatccgaagaagaaggcgtgcaggtctttggaagactcgcacgttggtcttataatgtatgatcgagatgtcaccctaatgccacatggtacaggcttatcgcggtcatggcgatcggacttgtaatttgcaacgatgggcaaaggatcgacgacatgccaaacattctgaacccgtagagatgttaacgatgacgaggatgaatatcccatgctcgctgccatagtatcaagtacaccgcgaataaggacgcgtccaacatcgttatatgcacacaatgggctacacgtgactaacacccccgaatattagtcatatgtgagtttcagtctggctcccatatagcctgtagactatttgtggtttaagtgtgaacgaggcgctgtgaacgagactcgggccgattgtaagaacaagcaagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 4 - DNA, 7693 bp, synthetic construct
  source 1..7693: mol_type = other DNA
accgccagatatccaaagtacagcacgatgttgcacggacgactttgcagtcaccagccttcctttccacccccccaccaacaaaatgtttatcgtaggacccatatccgtaataaggatgggtctggcagcaaccccataggcgcctcggcgtggtagttctcgaggatacatccaaagaggttgagtattctctctacacttcttgttaaatggaaagtgcatttgcttgttcttacaatcggcccgagtctcgttcacagcgcctcgttcacacttaaaccacaaatagtctacaggctatatgggagccagactgaaactcacatatgactaatattcgggggtgttagtcacgtgtagcccattgtgtgcatataacgatgttggacgcgtccttattcgcggtgtacttgatactatggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgtcgaccctgcaggtctagaggatccgaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccgcggtcattagatgcagatgttgcaacggcaagaaccgttagacatagcccagaacaggaaagcagccagacccatagccagaaccagagaagaagcaacggtagagtagatggtcaggattttgtaggtaccttcagattccagtttaacaccttcgattttctgacgttccagacgagattcttctttgtatttcccacggttgtaggtaccgttacggatggtttccatgcactggtcgtcgcatttgtggtacagttcgaagcaacctttaccgtcttccatagcgttagaacccagagcacgtttaactttgttgtacaggttgttaacgttagcgtcgtgttcgtccagggttttctggttttccagcagaaccagcagttcagcgttgtaagcccaaacgtcctggatctggtcgtcgattttgttgttgatcatgttcagacgggtttcaacttcagagaattcgtggtcgatgatttcgtactgtttgttcattttgtcaacgatgttgttaactttagaggtgattttgtcaacagctttctgggtagaatcacggtcagcagccataccaacaccctggtcgttagagtgctggaaaccgtaccaaccagcaaccagacccggccaaccaccttcgatgaaaccagcgatagcaccgaacagaccacgggaagacctagccggaacgttacgcagaccgatagccagtttcagagatttaacaccgatgtatttcgggcaagtaccgaaagcgtatttagagatgttgtggaacggcagggtagagttcagaccacctttttcggtctggcactgaacaacgcagttaccagagttcagatcggttttcaggatacgaccgtgagattcaccagacagaacgtgaccgaaccacggagcgatcaggttaccgttagaacgaacacgcagggtctgacccggtttcagaacagaccagtagtagttgatacgaccgatcagaccgttaaccagcggacgcggaccgatcagcggtttgaaggtacggtccaggttttcggtggtaacagaggtggtggtgtcggtacgggtgtacaggttggtctgagcggtgtcggtcggcgggtggtggataccccaaacgaacaggatgtctttaccacggttgttggtgaactgagcgtcctgaaccgggtacagaccgtttttctgggtcagccaacgcatgttacggtagaaagagtcagagcaagatttagaggtaccggtgtaggtaacgttccagatggtgtccgggaagatctggatacgctggtaagaagaagaagaagagaacagggtacgcagttcttccaggttttcaacgttacccgggtagcaggtaccgttaacagcagacggacgttcaacgatgtaagaccattcacgaccacccagcagcaggtcgcaagacgggttaccgtagatcagaccttcaatagtgcaggtgttcaggatcagcgggtgacccaggttggtagcgcacagcataccgttgtgttcggtgtgcagcagttctttagcgtgggtaaccggaacgttggtttcggtcagggtgtcaacggtttcggtagagttggtagactggtgaccgatgcagattttgtcagcgttagaggtggtaaccagcagcaggataatcatcaggctaattggttccatggtattatcatcgtgtttttcaaaggaaaaccacgtccccgtggttcggggggcctagacgttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggttacctccttatagcccggattatgtctttgaagccaaatgctcctgcaatcttcaggggagagttgaggtcggccacctccatgaagtactcacgaaaatcagtgtactcccttgttggccagacggtcttgatgccaagacggtccctctcactcagtatcaattttgtgtagttcatggctcctgggtcaaatcggccgtattctgtaaccaggttctttgctagttcaggatttggaatcagctcgaagttactcaccccagcgaccgtaacgacggatcctgttgccactctttcgtaggctactagtgtgacgggacggagggcccctggatagttgccaccatggatcgtcactgctaggctcccacttgccgaccatgacatctgatcccctgcctgaccaccacttttggaggtcactatctccagtttgatggatgtgattggctgggttatctcattggttggaatgacaagattgaatggcataagattgtcggtgccggccgtcagcccattatctgcggctacagctctggtgattaccgcagtcccatcaaagcctataaggtagatggtggcgcccagtacaaggccttggacgcttgtttgaaacacgagctctcccccaatgctgaggcttgtgatagcatcaatgttggctgagaacagtgtgattgttaccccacctggttggtactgtgatgagaattggtaatcatcggctgcagttatggtgtagactctgggcctgtcactgctgtcgcatgtagctaccatttttgggtcaagccctatagcgggaatggggtcaccaagcctcacatacccaagatcatatgatgtgggtaggctgaggacagtgaccccttcccctaccaggacattcccaattttgtcgttgatgttggctgttgcagacatcaacccattgtagctaacatctgtcagttcactcaggcttccttggaaggtcacggcgtttatggtgccgtttagtgcataaacgccaccagggagtgtgcttgacctcactgtgagactccgactcactagtctgcagtagttgtagctggccggtaggttctgggcagtcaggagcatctgatcgaacttgtagttcccattgctctgcagtgtgtagtgagcacccacaattgagccagggaatccagggaaaaagacaattagccctgaccctgtgtcccccacagtcaaattgtaggtcgaggtctctgacctgagagtgtgcttctccagggtgtcgtccggaatggacgccggtccggttgttggcatcagaaggctccgtatgaacggaacaatctgttgggtttgatcttgcaggtttgtcatcgctgcgatcgacgagttctagagcggccgcctgcagcgaggagctctgcgttctacggtggtcagaccgaagactgcgacggtaccgacgctggtcgcgcctcttatacccacgtagaacgcagctcagccaatagaatgcgtgccaatatggaatttccaggggaaaaccggggcggtgttacgttttggctgccctttcacttcccattgacgtgtattggctcgagaacggtactttcccattaatcagctatgggaaagtaccgtttaaaggtcacgttgcattagtttcaatagcccattgacgtcaatggtgggaaagtacatggcgttttaattaaattggctggaaaaacccaatgactcacccctattgaccttatgtacgtgccaataatgggaaaaacccattgactcaccccctattgaccttttgtactgggcaaaacccaatggaaagtccctattgactcagtgtacttggctccaatgggactttcctgttgattcacccctattgaccttatgtactgggcaaaacccattggaaagtccctaatgactcagtatacgtgccagtaatgggaaaaacccattggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaatgggtttttcccattgactcatcccctattgaccttatgtactgggcaaaacccaatggaaagtccctattgacgcagtgtacttggctccaatgggactttcctgttgattcaccccctattgaccttatgtactgggcagaatacaatggaaagtccctattgactcacccacattgaccttatatgcttgccaacaatggaaaaacccattggaaagtccctattgagtcagtgtacttggcagcaatgggtttttcccattggctcacctcccattgacccaatgtacttgggcaaaacccagtggaaagtcccatttgactcagtgtgcttgccagtaatgggaaaaacccattggcttacctcccattgacccaatgtacttgggtaaaggccattgagtcaccacccctatgctgggaaatggtgaacgccccctatgtggaaagtccctatgggccgccattagagtgcatgaccgtgctgattcatatgccatatgagtgtattagggggctttccgcttgggaaattgggtaaaaagtccccgtattactcacatagggggcgtttggctttgcaaattaggggatttcagtgcatttggcattaaaaactattggttctagtcataaaacgggcggagttgggggatccactagtgaattcgtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccattatcttcagtggagcgtagacgggcgaggctaattatgtgacatagcaacactgcatgtatgtttttataaatcaataagagtacataatttattacgtatcatttccgtttgtaatatactgtatacatcatccacactattagtcagcactagcgcgcgggcgcacgttacaatagcagcgtgcccgttatctatattgtccgatatttacacataacatttcatcgacatgattaaatacctaagtactgcacacagatgtttaatgtatatcgtcatataaattatatcgctaggacagacccaaacgacctttatcccaaacagtcagatcctcttctcaagtgtcgatttctgttatggaatatgcataccctggcccagaaattgcacgcacgagcgtagtgaatgcgtcattggttttacatttaaaggctaaatgcacaaattctttagacgacagcacatcgttaaatagcatctctagcgttcttatgaatgctaagcattggagtcctcctggtcggccacaataacagctgagtatcataccctgagctccggggttgtcgcacatagcggattcgtataaacataggattttccgcgaatccatcagttgcaaaaatctgttaggctccatcaacaacgctggatttacttcagatccacgcgtaaagtaatggtgctcgaataccgtttttagagttgtcggcatttcaaggaacaaaga

SEQ ID NO: 5 - protein, 553 aa, Avian orthoavulavirus 1
MGSKPSTRIPAPLMLITRIMLILGCIRPTSSLDGRPLAAAGIVVTGDKAVNVYTSSQTGSIIVKLLPNMPRDKEACAKAPLEAYNRTLTTLLTPLGDSIRKIQGSVSTSGGGKQGRLIGAVIGSVALGVATAAQITAAAALIQANQNAANILRLKESIAATNEAVHEVTDGLSQLSVAVGKMQQFVNDQFNNTARELDCIKITQQVGVELNLYLTELTTVFGPQITSPALTQLTIQALYNLAGGNMDYLLTKLGIGNNQLSSLIGSGLITGYPILYDSQTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALKGSVIANCKITTCRCTDPPGIISQNYGEAVSLIDRHSCNVLSLDGITLRLSGEFDATYQKNISILDSQVIVTGNLDISTELGNVNNSISNALDRLAESNSKLEKVNVRLTSTSALITYIVLTVISLVFGALSLVLACYLMYKQKAQQKTLLWLGNNTLDQMRATTRA

SEQ ID NO: 6 - DNA, 10507 bp, synthetic construct
  source 1..10507: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcagggtatgatactcagctgttattgtggccgaccaggaggactccaatgcttagcattcataagaacgctagagatgctatttaacgatgtgctgtcgtctaaagaatttgtgcatttagcctttaaatgtaaaaccaatgacgcattcactacgctcgtgcgtgcaatttctgggccagggtatgcatattccataacagaaatcgacacttgagaagaggatctgactgtttgggataaaggtcgtttgggtctgtcctagcgatataatttatatgacgatatacattaaacatctgtgtgcagtacttaggtatttaatcatgtcgatgaaatgttatgtgtaaatatcggacaatatagataacgggcacgctgctattgtaacgtgcgcccgcgcgctagtgctgactaatagtgtggatgatgtatacagtatattacaaacggaaatgatacgtaataaattatgtactcttattgatttataaaaacatacatgcagtgttgctatgtcacataattagcctcgcccgtctacgctccactgaagataatgggctcccgctgttcaaaaaaatcagcgtgcgtcgataagactttggtgcagtctcttcggggtcgcaatttagatttgccgcatggagggtatctggggatttttgccaatgctggagcgacgactgtacgattcgtcccatcgggatctagcagaccaatgatgttgacacacatcggccatgcatgtacggacggtctattgcgcgagtttgttattttcgaaggacaagatggaagtgtatatggaaccgacaataatgttagtttgcatttcttagggcggaatctacatgatatcttatccaagcggggtatgagccagagagatgtgatggtcataaagggtaaattttttagatctgaaataacgcagttgcccaaacaacgatcgcgattaaaagaaaaatcggatggttcaattaggacatgcatggattctgtgcgcataaaccataaccgcagcactgttgggcacttcggtaactcaaatgcgaagcgttgcacgtctgcgataactacgcctactatgcacattgttactcctgcatcttaaaaatatatcctgtagtaattttcacagcaatgtcataacatcatctcgctaaagaatgacctgggattggagaagtaatgaatatttgcaaccaatgcattgaataaactaacattaaacgaattcactagtggatcccccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgcatgggctccaaaccttctaccaggatcccagcacctctgatgctgatcacccggattatgctgatattgggctgtatccgtccgacaagctctcttgacggcaggcctcttgcagctgcaggaattgtagtaacaggagataaggcagtcaatgtatacacttcgtctcagacagggtcaatcatagtcaagttgctcccgaatatgcccagggataaggaggcgtgtgcaaaagccccattagaggcatataacagaacactgactactttgctcactcctcttggcgactccatccgcaagatccaagggtctgtgtccacatctggaggaggcaagcaaggccgcctgataggtgctgttattggcagtgtagctcttggggttgcaacagcggcacagataacagcagctgcggccctaatacaagccaaccagaatgccgccaacatcctccggcttaaggagagcattgctgcaaccaatgaagctgtgcatgaagtcaccgacggattatcacaactatcagtggcagttgggaagatgcagcagtttgtcaatgaccagtttaataatacggcgcgagaattggactgtataaaaatcacacaacaggttggtgtagaactcaacctatacctaactgaattgactacagtattcgggccacagatcacctcccctgcattaactcagctgaccatccaggcactttataatttagctggtggcaatatggattacttattaactaagttaggtatagggaacaatcaactcagctcgttaattggtagcggcctgatcactggttaccctatactgtatgactcacagactcaactcttgggcatacaagtgaatttaccctcagtcgggaacttaaataatatgcgtgccacctatttggagaccttatctgtaagtacaaccaaaggatatgcctcagcacttgtcccgaaagtagtgacacaagtcggttccgtgatagaagagcttgacacctcatactgtatagagtccgatctggatttatattgtactagaatagtgacattccccatgtccccaggtatttattcctgtttgagcggcaacacatcagcttgcatgtattcaaagactgaaggcgcactcactacgccgtatatggcccttaaaggctcagttattgccaattgtaaaataacaacatgtagatgtacagaccctcctggtatcatatcgcaaaattatggagaagctgtatccctgatagatagacattcgtgcaatgtcttatcattagacgggataactctaaggctcagtggggaatttgatgcaacttatcaaaagaacatctcaatactagattctcaagtcatcgtgacaggcaatcttgatatatcaactgaacttggaaacgtcaacaattcaatcagcaatgccttggataggttggcagaaagcaacagcaagctagaaaaagtcaatgtcagactaaccagcacatctgctctcattacctatattgttctaactgtcatttctctagttttcggtgcacttagtctggtgttagcgtgttacctgatgtacaaacagaaggcacaacaaaagaccttgctatggcttgggaataataccctcgatcagatgagagccactacaagagcatgagcggccgccccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggaaccaattagcctgatgattatcctgctgctggttaccacctctaacgctgacaaaatctgcatcggtcaccagtctaccaactctaccgaaaccgttgacaccctgaccgaaaccaacgttccggttacccacgctaaagaactgctgcacaccgaacacaacggtatgctgtgcgctaccaacctgggtcacccgctgatcctgaacacctgcactattgaaggtctgatctacggtaacccgtcttgcgacctgctgctgggtggtcgtgaatggtcttacatcgttgaacgtccgtctgctgttaacggtacctgctacccgggtaacgttgaaaacctggaagaactgcgtaccctgttctcttcttcttcttcttaccagcgtatccagatcttcccggacaccatctggaacgttacctacaccggtacctctaaatcttgctctgactctttctaccgtaacatgcgttggctgacccagaaaaacggtctgtacccggttcaggacgctcagttcaccaacaaccgtggtaaagacatcctgttcgtttggggtatccaccacccgccgaccgacaccgctcagaccaacctgtacacccgtaccgacaccaccacctctgttaccaccgaaaacctggaccgtaccttcaaaccgctgatcggtccgcgtccgctggttaacggtctgatcggtcgtatcaactactactggtctgttctgaaaccgggtcagaccctgcgtgttcgttctaacggtaacctgatcgctccgtggttcggtcacgttctgtctggtgaatctcacggtcgtatcctgaaaaccgatctgaactctggtaactgcgttgttcagtgccagaccgaaaaaggtggtctgaactctaccctgccgttccacaacatctctaaatacgctttcggtacttgcccgaaatacatcggtgttaaatctctgaaactggctatcggtctgcgtaacgttccggctaggtcttcccgtggtctgttcggtgctatcgctggtttcatcgaaggtggttggccgggtctggttgctggttggtacggtttccagcactctaacgaccagggtgttggtatggctgctgaccgtgattctacccagaaagctgttgacaaaatcacctctaaagttaacaacatcgttgacaaaatgaacaaacagtacgaaatcatcgaccacgaattctctgaagttgaaacccgtctgaacatgatcaacaacaaaatcgacgaccagatccaggacgtttgggcttacaacgctgaactgctggttctgctggaaaaccagaaaaccctggacgaacacgacgctaacgttaacaacctgtacaacaaagttaaacgtgctctgggttctaacgctatggaagacggtaaaggttgcttcgaactgtaccacaaatgcgacgaccagtgcatggaaaccatccgtaacggtacctacaaccgtgggaaatacaaagaagaatctcgtctggaacgtcagaaaatcgaaggtgttaaactggaatctgaaggtacctacaaaatcctgaccatctactctaccgttgcttcttctctggttctggctatgggtctggctgctttcctgttctgggctatgtctaacggttcttgccgttgcaacatctgcatctaatgaccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacaattattttatttaataacatatagcccaaagacctctatgaacatttagtttcccgtatactcaacggcgcgtgtacacacgcatctctttgcatagcgatgaagtttgttcggcagcagaaaatgcagatatccaacaatctggagaaaacttatcatcacagtggcagtggaaacataccccctctatattcatggtataattatcgtctacagcgtccaggatagtggcgtgagaaaatggagatctgcagccctcctttccatggcatgccgctttattgttcattaaacgcacaatggtctcaacgccagatatgggcatagattctgaagaacccgttgacaatccgaagaagaaggcgtgcaggtctttggaagactcgcacgttggtcttataatgtatgatcgagatgtcaccctaatgccacatggtacaggcttatcgcggtcatggcgatcggacttgtaatttgcaacgatgggcaaaggatcgacgacatgccaaacattctgaacccgtagagatgttaacgatgacgaggatgaatatcccatgctcgctgccatagtatcaagtacaccgcgaataaggacgcgtccaacatcgttatatgcacacaatgggctacacgtgactaacacccccgaatattagtcatatgtgagtttcagtctggctcccatatagcctgtagactatttgtggtttaagtgtgaacgaggcgctgtgaacgagactcgggccgattgtaagaacaagcaaatgcactttccatttaacaagaagtgtagagagaatactcaacctctttggatgtatcctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 7 - DNA, 7972 bp, synthetic construct
  source 1..7972: mol_type = other DNA
accgccagatatccaaagtacagcacgatgttgcacggacgactttgcagtcaccagccttcctttccacccccccaccaacaaaatgtttatcgtaggacccatatccgtaataaggatgggtctggcagcaaccccataggcgcctcggcgtggtagttctcgaggatacatccaaagaggttgagtattctctctacacttcttgttaaatggaaagtgcatttgcttgttcttacaatcggcccgagtctcgttcacagcgcctcgttcacacttaaaccacaaatagtctacaggctatatgggagccagactgaaactcacatatgactaatattcgggggtgttagtcacgtgtagcccattgtgtgcatataacgatgttggacgcgtccttattcgcggtgtacttgatactatggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgtcgactctagaggatccgaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccgcggtcattagatgcagatgttgcaacggcaagaaccgttagacatagcccagaacaggaaagcagccagacccatagccagaaccagagaagaagcaacggtagagtagatggtcaggattttgtaggtaccttcagattccagtttaacaccttcgattttctgacgttccagacgagattcttctttgtatttcccacggttgtaggtaccgttacggatggtttccatgcactggtcgtcgcatttgtggtacagttcgaagcaacctttaccgtcttccatagcgttagaacccagagcacgtttaactttgttgtacaggttgttaacgttagcgtcgtgttcgtccagggttttctggttttccagcagaaccagcagttcagcgttgtaagcccaaacgtcctggatctggtcgtcgattttgttgttgatcatgttcagacgggtttcaacttcagagaattcgtggtcgatgatttcgtactgtttgttcattttgtcaacgatgttgttaactttagaggtgattttgtcaacagctttctgggtagaatcacggtcagcagccataccaacaccctggtcgttagagtgctggaaaccgtaccaaccagcaaccagacccggccaaccaccttcgatgaaaccagcgatagcaccgaacagaccacgggaagacctagccggaacgttacgcagaccgatagccagtttcagagatttaacaccgatgtatttcgggcaagtaccgaaagcgtatttagagatgttgtggaacggcagggtagagttcagaccacctttttcggtctggcactgaacaacgcagttaccagagttcagatcggttttcaggatacgaccgtgagattcaccagacagaacgtgaccgaaccacggagcgatcaggttaccgttagaacgaacacgcagggtctgacccggtttcagaacagaccagtagtagttgatacgaccgatcagaccgttaaccagcggacgcggaccgatcagcggtttgaaggtacggtccaggttttcggtggtaacagaggtggtggtgtcggtacgggtgtacaggttggtctgagcggtgtcggtcggcgggtggtggataccccaaacgaacaggatgtctttaccacggttgttggtgaactgagcgtcctgaaccgggtacagaccgtttttctgggtcagccaacgcatgttacggtagaaagagtcagagcaagatttagaggtaccggtgtaggtaacgttccagatggtgtccgggaagatctggatacgctggtaagaagaagaagaagagaacagggtacgcagttcttccaggttttcaacgttacccgggtagcaggtaccgttaacagcagacggacgttcaacgatgtaagaccattcacgaccacccagcagcaggtcgcaagacgggttaccgtagatcagaccttcaatagtgcaggtgttcaggatcagcgggtgacccaggttggtagcgcacagcataccgttgtgttcggtgtgcagcagttctttagcgtgggtaaccggaacgttggtttcggtcagggtgtcaacggtttcggtagagttggtagactggtgaccgatgcagattttgtcagcgttagaggtggtaaccagcagcaggataatcatcaggctaattggttccatggtattatcatcgtgtttttcaaaggaaaaccacgtccccgtggttcggggggcctagacgttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttagggggggggggggcggccgctcatgctcttgtagtggctctcatctgatcgagggtattattcccaagccatagcaaggtcttttgttgtgccttctgtttgtacatcaggtaacacgctaacaccagactaagtgcaccgaaaactagagaaatgacagttagaacaatataggtaatgagagcagatgtgctggttagtctgacattgactttttctagcttgctgttgctttctgccaacctatccaaggcattgctgattgaattgttgacgtttccaagttcagttgatatatcaagattgcctgtcacgatgacttgagaatctagtattgagatgttcttttgataagttgcatcaaattccccactgagccttagagttatcccgtctaatgataagacattgcacgaatgtctatctatcagggatacagcttctccataattttgcgatatgataccaggagggtctgtacatctacatgttgttattttacaattggcaataactgagcctttaagggccatatacggcgtagtgagtgcgccttcagtctttgaatacatgcaagctgatgtgttgccgctcaaacaggaataaatacctggggacatggggaatgtcactattctagtacaatataaatccagatcggactctatacagtatgaggtgtcaagctcttctatcacggaaccgacttgtgtcactactttcgggacaagtgctgaggcatatcctttggttgtacttacagataaggtctccaaataggtggcacgcatattatttaagttcccgactgagggtaaattcacttgtatgcccaagagttgagtctgtgagtcatacagtatagggtaaccagtgatcaggccgctaccaattaacgagctgagttgattgttccctatacctaacttagttaataagtaatccatattgccaccagctaaattataaagtgcctggatggtcagctgagttaatgcaggggaggtgatctgtggcccgaatactgtagtcaattcagttaggtataggttgagttctacaccaacctgttgtgtgatttttatacagtccaattctcgcgccgtattattaaactggtcattgacaaactgctgcatcttcccaactgccactgatagttgtgataatccgtcggtgacttcatgcacagcttcattggttgcagcaatgctctccttaagccggaggatgttggcggcattctggttggcttgtattagggccgcagctgctgttatctgtgccgctgttgcaaccccaagagctacactgccaataacagcacctatcaggcggccttgcttgcctcctccagatgtggacacagacccttggatcttgcggatggagtcgccaagaggagtgagcaaagtagtcagtgttctgttatatgcctctaatggggcttttgcacacgcctccttatccctgggcatattcgggagcaacttgactatgattgaccctgtctgagacgaagtgtatacattgactgccttatctcctgttactacaattcctgcagctgcaagaggcctgccgtcaagagagcttgtcggacggatacagcccaatatcagcataatccgggtgatcagcatcagaggtgctgggatcctggtagaaggtttggagcccatgcggccgcctgcagcgaggagctctgcgttctacggtggtcagaccgaagactgcgacggtaccgacgctggtcgcgcctcttatacccacgtagaacgcagctcagccaatagaatgcgtgccaatatggaatttccaggggaaaaccggggcggtgttacgttttggctgccctttcacttcccattgacgtgtattggctcgagaacggtactttcccattaatcagctatgggaaagtaccgtttaaaggtcacgttgcattagtttcaatagcccattgacgtcaatggtgggaaagtacatggcgttttaattaaattggctggaaaaacccaatgactcacccctattgaccttatgtacgtgccaataatgggaaaaacccattgactcaccccctattgaccttttgtactgggcaaaacccaatggaaagtccctattgactcagtgtacttggctccaatgggactttcctgttgattcacccctattgaccttatgtactgggcaaaacccattggaaagtccctaatgactcagtatacgtgccagtaatgggaaaaacccattggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaatgggtttttcccattgactcatcccctattgaccttatgtactgggcaaaacccaatggaaagtccctattgacgcagtgtacttggctccaatgggactttcctgttgattcaccccctattgaccttatgtactgggcagaatacaatggaaagtccctattgactcacccacattgaccttatatgcttgccaacaatggaaaaacccattggaaagtccctattgagtcagtgtacttggcagcaatgggtttttcccattggctcacctcccattgacccaatgtacttgggcaaaacccagtggaaagtcccatttgactcagtgtgcttgccagtaatgggaaaaacccattggcttacctcccattgacccaatgtacttgggtaaaggccattgagtcaccacccctatgctgggaaatggtgaacgccccctatgtggaaagtccctatgggccgccattagagtgcatgaccgtgctgattcatatgccatatgagtgtattagggggctttccgcttgggaaattgggtaaaaagtccccgtattactcacatagggggcgtttggctttgcaaattaggggatttcagtgcatttggcattaaaaactattggttctagtcataaaacgggcggagttgggggatccactagtgaattcgtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccattatcttcagtggagcgtagacgggcgaggctaattatgtgacatagcaacactgcatgtatgtttttataaatcaataagagtacataatttattacgtatcatttccgtttgtaatatactgtatacatcatccacactattagtcagcactagcgcgcgggcgcacgttacaatagcagcgtgcccgttatctatattgtccgatatttacacataacatttcatcgacatgattaaatacctaagtactgcacacagatgtttaatgtatatcgtcatataaattatatcgctaggacagacccaaacgacctttatcccaaacagtcagatcctcttctcaagtgtcgatttctgttatggaatatgcataccctggcccagaaattgcacgcacgagcgtagtgaatgcgtcattggttttacatttaaaggctaaatgcacaaattctttagacgacagcacatcgttaaatagcatctctagcgttcttatgaatgctaagcattggagtcctcctggtcggccacaataacagctgagtatcataccctgagctccggggttgtcgcacatagcggattcgtataaacataggattttccgcgaatccatcagttgcaaaaatctgttaggctccatcaacaacgctggatttacttcagatccacgcgtaaagtaatggtgctcgaataccgtttttagagttgtcggcatttcaaggaacaaaga

SEQ ID NO: 8 - DNA, 1391 bp, Human betaherpesvirus 5
  source 1..1391: mol_type = genomic DNA
aactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcag

SEQ ID NO: 9 - DNA, 1686 bp, synthetic construct
  source 1..1686: mol_type = other DNA
atggaaccaattagcctgatgattatcctgctgctggttaccacctctaacgctgacaaaatctgcatcggtcaccagtctaccaactctaccgaaaccgttgacaccctgaccgaaaccaacgttccggttacccacgctaaagaactgctgcacaccgaacacaacggtatgctgtgcgctaccaacctgggtcacccgctgatcctgaacacctgcactattgaaggtctgatctacggtaacccgtcttgcgacctgctgctgggtggtcgtgaatggtcttacatcgttgaacgtccgtctgctgttaacggtacctgctacccgggtaacgttgaaaacctggaagaactgcgtaccctgttctcttcttcttcttcttaccagcgtatccagatcttcccggacaccatctggaacgttacctacaccggtacctctaaatcttgctctgactctttctaccgtaacatgcgttggctgacccagaaaaacggtctgtacccggttcaggacgctcagttcaccaacaaccgtggtaaagacatcctgttcgtttggggtatccaccacccgccgaccgacaccgctcagaccaacctgtacacccgtaccgacaccaccacctctgttaccaccgaaaacctggaccgtaccttcaaaccgctgatcggtccgcgtccgctggttaacggtctgatcggtcgtatcaactactactggtctgttctgaaaccgggtcagaccctgcgtgttcgttctaacggtaacctgatcgctccgtggttcggtcacgttctgtctggtgaatctcacggtcgtatcctgaaaaccgatctgaactctggtaactgcgttgttcagtgccagaccgaaaaaggtggtctgaactctaccctgccgttccacaacatctctaaatacgctttcggtacttgcccgaaatacatcggtgttaaatctctgaaactggctatcggtctgcgtaacgttccggctaggtcttcccgtggtctgttcggtgctatcgctggtttcatcgaaggtggttggccgggtctggttgctggttggtacggtttccagcactctaacgaccagggtgttggtatggctgctgaccgtgattctacccagaaagctgttgacaaaatcacctctaaagttaacaacatcgttgacaaaatgaacaaacagtacgaaatcatcgaccacgaattctctgaagttgaaacccgtctgaacatgatcaacaacaaaatcgacgaccagatccaggacgtttgggcttacaacgctgaactgctggttctgctggaaaaccagaaaaccctggacgaacacgacgctaacgttaacaacctgtacaacaaagttaaacgtgctctgggttctaacgctatggaagacggtaaaggttgcttcgaactgtaccacaaatgcgacgaccagtgcatggaaaccatccgtaacggtacctacaaccgtgggaaatacaaagaagaatctcgtctggaacgtcagaaaatcgaaggtgttaaactggaatctgaaggtacctacaaaatcctgaccatctactctaccgttgcttcttctctggttctggctatgggtctggctgctttcctgttctgggctatgtctaacggttcttgccgttgcaacatctgcatctaatga

SEQ ID NO: 10 - DNA, 1362 bp, Infectious bursal disease virus
  source 1..1362: mol_type = genomic DNA
atgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaaattgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtagccgcagataatgggctgacggccggcaccgacaatcttatgccattcaatcttgtcattccaaccaatgagataacccagccaatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgattccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaa

SEQ ID NO: 11 - DNA, 1661 bp, Avian orthoavulavirus 1
  source 1..1661: mol_type = genomic DNA
atgggctccaaaccttctaccaggatcccagcacctctgatgctgatcacccggattatgctgatattgggctgtatccgtccgacaagctctcttgacggcaggcctcttgcagctgcaggaattgtagtaacaggagataaggcagtcaatgtatacacttcgtctcagacagggtcaatcatagtcaagttgctcccgaatatgcccagggataaggaggcgtgtgcaaaagccccattagaggcatataacagaacactgactactttgctcactcctcttggcgactccatccgcaagatccaagggtctgtgtccacatctggaggaggcaagcaaggccgcctgataggtgctgttattggcagtgtagctcttggggttgcaacagcggcacagataacagcagctgcggccctaatacaagccaaccagaatgccgccaacatcctccggcttaaggagagcattgctgcaaccaatgaagctgtgcatgaagtcaccgacggattatcacaactatcagtggcagttgggaagatgcagcagtttgtcaatgaccagtttaataatacggcgcgagaattggactgtataaaaatcacacaacaggttggtgtagaactcaacctatacctaactgaattgactacagtattcgggccacagatcacctcccctgcattaactcagctgaccatccaggcactttataatttagctggtggcaatatggattacttattaactaagttaggtatagggaacaatcaactcagctcgttaattggtagcggcctgatcactggttaccctatactgtatgactcacagactcaactcttgggcatacaagtgaatttaccctcagtcgggaacttaaataatatgcgtgccacctatttggagaccttatctgtaagtacaaccaaaggatatgcctcagcacttgtcccgaaagtagtgacacaagtcggttccgtgatagaagagcttgacacctcatactgtatagagtccgatctggatttatattgtactagaatagtgacattccccatgtccccaggtatttattcctgtttgagcggcaacacatcagcttgcatgtattcaaagactgaaggcgcactcactacgccgtatatggcccttaaaggctcagttattgccaattgtaaaataacaacatgtagatgtacagaccctcctggtatcatatcgcaaaattatggagaagctgtatccctgatagatagacattcgtgcaatgtcttatcattagacgggataactctaaggctcagtggggaatttgatgcaacttatcaaaagaacatctcaatactagattctcaagtcatcgtgacaggcaatcttgatatatcaactgaacttggaaacgtcaacaattcaatcagcaatgccttggataggttggcagaaagcaacagcaagctagaaaaagtcaatgtcagactaaccagcacatctgctctcattacctatattgttctaactgtcatttctctagttttcggtgcacttagtctggtgttagcgtgttacctgatgtacaaacagaaggcacaacaaaagaccttgctatggcttgggaataataccctcgatcagatgagagccactacaagagcatg

SEQ ID NO: 12 - DNA, 563 bp, synthetic construct
  source 1..563: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 13 - DNA, 199 bp, synthetic construct
  source 1..199: mol_type = other DNA
ggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 14 - DNA, 66 bp, synthetic construct
  source 1..66: mol_type = other DNA
ggctccggcgccaccaacttctccctgctcaagcaggccggcgacgtggaggagaaccctggacct

SEQ ID NO: 15 - DNA, 22 bp, synthetic construct
  source 1..22: mol_type = other DNA
cgaacaaacttcatcgctatgc

SEQ ID NO: 16 - DNA, 22 bp, synthetic construct
  source 1..22: mol_type = other DNA
taactcaaatgcgaagcgttgc

SEQ ID NO: 17 - DNA, 20 bp, synthetic construct
  source 1..20: mol_type = other DNA
ctctgaagttgaaacccgtc

SEQ ID NO: 18 - DNA, 21 bp, synthetic construct
  source 1..21: mol_type = other DNA
agattgcaggagcatttggct

SEQ ID NO: 19 - DNA, 18 bp, synthetic construct
  source 1..18: mol_type = other DNA
atgcaattgttgttaact

SEQ ID NO: 20 - DNA, 18 bp, synthetic construct
  source 1..18: mol_type = other DNA
aactccgcccgttttatg

SEQ ID NO: 21 - DNA, 24 bp, synthetic construct
  source 1..24: mol_type = other DNA
tctttgttccttgaaatgccgaca

SEQ ID NO: 22 - DNA, 21 bp, synthetic construct
  source 1..21: mol_type = other DNA
tcatgtcgatgaaatgttatg

SEQ ID NO: 23 - DNA, 20 bp, synthetic construct
  source 1..20: mol_type = other DNA
tgcgtcgataagactttggt

SEQ ID NO: 24 - DNA, 20 bp, synthetic construct
  source 1..20: mol_type = other DNA
ttagtttgcatttcttaggg

SEQ ID NO: 25 - DNA, 20 bp, synthetic construct
  source 1..20: mol_type = other DNA
tgaatatttgcaaccaatgc

SEQ ID NO: 26 - DNA, 20 bp, synthetic construct
  source 1..20: mol_type = other DNA
tcagcacggtcatgcactct

SEQ ID NO: 27 - DNA, 22 bp, synthetic construct
  source 1..22: mol_type = other DNA
atgtactttcccaccattgacg

SEQ ID NO: 28 - DNA, 19 bp, synthetic construct
  source 1..19: mol_type = other DNA
aggcggccgctctagaacg

SEQ ID NO: 29 - DNA, 18 bp, synthetic construct
  source 1..18: mol_type = other DNA
accggccagctacaacta

SEQ ID NO: 30 - DNA, 18 bp, synthetic construct
  source 1..18: mol_type = other DNA
atgcgacagcagtgacag

SEQ ID NO: 31 - DNA, 18 bp, synthetic construct
  source 1..18: mol_type = other DNA
agataacccagccaatca

SEQ ID NO: 32 - DNA, 20 bp, synthetic construct
  source 1..20: mol_type = other DNA
actgagtgagagggaccgtc

SEQ ID NO: 33 - DNA, 20 bp, synthetic construct
  source 1..20: mol_type = other DNA
atcaggctaattggttccat

SEQ ID NO: 34 - DNA, 20 bp, synthetic construct
  source 1..20: mol_type = other DNA
atggaaccaattagcctgat

SEQ ID NO: 35 - DNA, 20 bp, synthetic construct
  source 1..20: mol_type = other DNA
atggaaccaattagcctgat

SEQ ID NO: 36 - DNA, 20 bp, synthetic construct
  source 1..20: mol_type = other DNA
acgttctgtctggtgaatct

SEQ ID NO: 37 - DNA, 18 bp, synthetic construct
  source 1..18: mol_type = other DNA
acacgcgccgttgagtat

SEQ ID NO: 38 - DNA, 18 bp, synthetic construct
  source 1..18: mol_type = other DNA
atactcaacggcgcgtgt

SEQ ID NO: 39 - DNA, 18 bp, synthetic construct
  source 1..18: mol_type = other DNA
atccgaagaagaaggcgt

SEQ ID NO: 40 - DNA, 20 bp, synthetic construct
  source 1..20: mol_type = other DNA
accgccagatatccaaagta

SEQ ID NO: 41 - DNA, 20 bp, synthetic construct
  source 1..20: mol_type = other DNA
cgtcaacaattcaatcagca

SEQ ID NO: 42 - DNA, 20 bp, synthetic construct
  source 1..20: mol_type = other DNA
tatgcccagggataaggagg

SEQ ID NO: 43 - DNA, 18 bp, synthetic construct
  source 1..18: mol_type = other DNA
acttcatgcacagcttca

SEQ ID NO: 44 - DNA, 18 bp, synthetic construct
  source 1..18: mol_type = other DNA
ctggtggcaatatggatt

SEQ ID NO: 45 - DNA, 20 bp, synthetic construct
  source 1..20: mol_type = other DNA
atgtgttgccgctcaaacag